(19) United States
(12) Patent Application Publication
(10) Pub. No.: US 2015/0080381 A1
(43) Pub. Date: Mar. 19, 2015

(54) METHODS OF TREATING ALOPECIA AND ACNE

(71) Applicant: Ironwood Pharmaceuticals, Inc., Cambridge, MA (US)

(72) Inventors: Mark G. Currie, Sterling, MA (US); Yueh-tyng Chien, Newton, MA (US)

(21) Appl. No.: 14/391,451

(22) PCT Filed: Apr. 12, 2013
(86) PCT No.: PCT/US13/36383

§ 371 Date: Oct. 9, 2014

Related U.S. Application Data
(60) Provisional application No. 61/623,125, filed on Apr. 12, 2012.

(51) Int. Cl. C07D 401/04; C07D 231/12; C07D 209/14; C07D 207/34

(52) U.S. Cl. CPC C07D 401/04; C07D 207/34; C07D 231/12; C07D 209/14
USPC 514/217.08; 514/422; 514/343; 514/427; 514/235.5; 514/406; 514/414; 514/254.09; 514/235.2

(57) ABSTRACT

Method of treating alopecia and acne with are disclosed. The compounds fall within described by formula I or II:

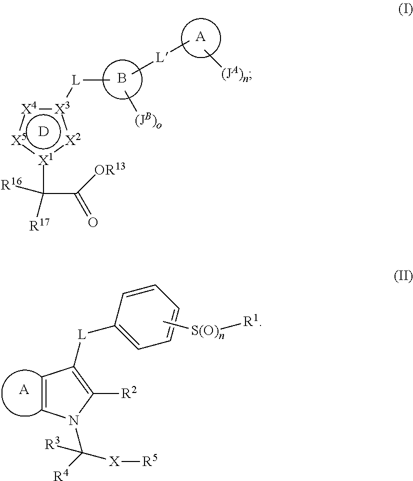

### RELATED APPLICATION

This application claims the benefit of U.S. Provisional Application No. 61/623,125, filed Apr. 12, 2012, the entire teachings of which are incorporated herein by reference.

### FIELD OF THE INVENTION

This disclosure relates to methods of treating alopecia and acne.

### BACKGROUND

Excessive or abnormal hair loss (alopecia) affects million of men and woman. Androgenetic alopecia or common male pattern baldness (MPB) accounts vast majority of incidents of hair loss in men, with approximately 80% of Caucasian men experiencing some degree of androgenetic alopecia by the age of 80. Acne is another widespread problem afflicting millions of men and women. Acne may be an acute or life-long chronic problem.

Despite being widespread problems, the number of effective treatments for hair loss are few and do not work for all patients. More acne therapies are available; however, such therapies do not resolve all incidences of acne for all patients. In addition to the physical symptoms of alopecia and acne, those with alopecia or acne may also suffer psychological problems as well, often feeling embarrassed and unhappy about their physical appearance.

Accordingly, there is a need to develop new treatments for both alopecia and acne.

### SUMMARY

In a first aspect, the present invention provides a method for preventing or lessening the severity of or treating a patient suffering from alopecia or acne comprising administering to said patient a therapeutically effective amount of a compound of Formula I, or a pharmaceutically acceptable salt thereof; or a pharmaceutical composition comprising the compound of Formula I, or a pharmaceutically acceptable salt thereof, and a pharmaceutically acceptable carrier; wherein the compound of Formula I is represented by the following structural formula:

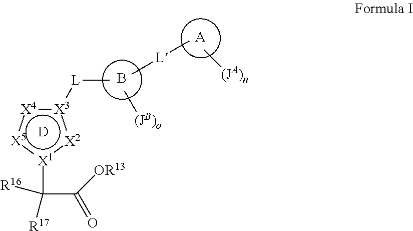

wherein:

Ring A is a monocyclic or bicyclic ring selected from a 6 to 10-membered aryl, a 5 to 10-membered heteroaryl, a C₃₋₁₀ cycloaliphatic and a 4 to 10-membered heterocycle; wherein said heteroaryl or heterocycle contains from 0 to 3 ring heteroatoms independently selected from N, O and S.

Ring B is a monocyclic ring selected from a phenyl and a 5 to 6-membered heteroaryl, wherein said heteroaryl contains up to three ring heteroatoms independently selected from N, O, and S.

Ring D is a 5-membered heteroaryl; wherein x¹ is selected from N and C; x² is selected from N and C—R²; x³ is selected from N and C; x⁴ is selected from N and C—R⁴; and x⁵ is selected from N and C—R⁵; provided that at least one of x¹ or x³ is N, but both are not simultaneously N.

R² is selected from —H, a halogen, —NO₂, —CN, a C₁₋₆ aliphatic radical, a C₁₋₆ alkoxy and a cyclopropyl ring, wherein R² is independently substituted with from 0 to 3 instances of R^{A}; wherein each R^{A} is independently selected from a halogen, —OH, a C₁₋₂ alkoxy and a C₁₋₂ haloalkoxy.

R⁴ is selected from a halogen, —NO₂, —CN, —R⁶, —OR⁶, —C(O)R⁶, —C(O)OR⁶, —N(R⁶)₂, —S(O)ₚR⁶, —S(O)₂N(R⁶)₂, —NR⁶S(O)₂R⁶, —C(O)N(R⁶)₂ and —NR⁶C(O)R⁶.

R⁵ is selected from a halogen, —NO₂, —CN, —R⁶, —OR⁶, —C(O)R⁶, —C(O)OR⁶, —N(R⁶)₂, —S(O)ₚR⁶, —S(O)₂N(R⁶)₂, —NR⁶S(O)₂R⁶, —C(O)N(R⁶)₂ and —NR⁶C(O)R⁶.

p is an integer selected from 0, 1 and 2.

Each R⁶ is independently selected from —H, a C₁₋₆ aliphatic radical, and a monocyclic or bicyclic ring; wherein: the ring is selected from a 6 to 10-membered aryl, a 5 to 10-membered heteroaryl, a C₃₋₁₀ cycloaliphatic and a 4 to 10-membered heterocycle; when R⁶ is a C₁₋₆ aliphatic radical, it is independently substituted with from 0 to 6 instances of R⁷; when R⁶ is a non-aromatic ring or a heteroaryl, it is independently substituted with from 0 to 6 instances of R⁸; and when R⁶ is an aryl, it is independently substituted with from 0 to 6 instances of R^{8′}.

Each R⁷ is independently selected from a halogen, —CN, oxo, —OR⁹, —R¹⁰, —C(O)R⁹, —C(O)OR⁹, —S(O)ₘR⁹, —N(R⁹)₂, —S(O)₂N(R⁹)₂, —NR⁹S(O)₂R⁹, —C(O)N(R⁹)₂ and —NR⁹C(O)R⁹.

Each R⁸ is independently selected from a halogen, —CN, —NO₂, oxo, a C₁₋₆ aliphatic radical, —R¹⁰, —C(O)R⁹, —C(O)OR⁹, —OR⁹, —S(O)ₘR⁹, —N(R⁹)₂, —S(O)₂N(R⁹)₂, —NR⁹S(O)₂R⁹, —C(O)N(R⁹)₂ and —NR⁹C(O)R⁹.

Each R⁸ is independently selected from a halogen, —CN, —NO₂, a C₁₋₆ aliphatic radical, —R¹⁰, —C(O)R⁹, —C(O)OR⁹, —OR⁹, —S(O)ₘR⁹, —N(R⁹)₂, —S(O)₂N(R⁹)₂, —NR⁹S(O)₂R⁹, —C(O)N(R⁹)₂ and —NR⁹C(O)R⁹.

Each R⁹ is independently selected from hydrogen, a C₁₋₆ aliphatic radical, and a monocyclic or bicyclic ring, wherein the ring is selected from a 6 to 10-membered aryl, a 5 to 10-membered heteroaryl, a C₃₋₁₀ cycloaliphatic and a 4 to 10-membered heterocycle; when R⁹ is a C₁₋₆ aliphatic radical, it is independently substituted with from 0 to 6 instances of R¹¹; and when R⁹ is a ring, it is independently substituted with from 0 to 3 instances of R¹².

Each R¹⁰ is a monocyclic or bicyclic ring independently selected from a 6 to 10-membered aryl, a 5 to 10-membered heteroaryl, a C₃₋₁₀ cycloaliphatic and a 4 to 10-membered heterocycle; and R¹⁰ is independently substituted with from 0 to 3 instances of R¹².

Each R¹¹ is independently selected from a halogen, —CN, —OH, a C₁₋₄ alkoxy and a C₁₋₄ haloalkoxy.

Each R¹² is independently selected from a halogen, —CN, —OH, a C₁₋₄ alkyl, a C₁₋₄ haloalkyl, a C₁₋₄ alkoxy and a C₁₋₄ haloalkoxy.

R¹³ is selected from —H, a C₁₋₆ aliphatic radical, and a monocyclic or bicyclic ring, wherein the ring is selected from a 6 to 10-membered aryl, a 5 to 10-membered heteroaryl, a C₃₋₁₀ cycloaliphatic and a 4 to 10-membered heterocycle; and when R¹³ is a C₁₋₆ aliphatic radical, it is independently substituted with from 0 to 6 instances of R¹⁴; when R¹³ is a non-aromatic ring or a heteroaryl, it is independently substituted with from 0 to 6 instances of R¹⁵; and when R¹³ is an aryl, it is independently substituted with from 0 to 6 instances of R^{15′}.

Each R¹⁴ is independently selected from a halogen, —CN, oxo, —OR⁹, —R¹⁰, —C(O)R⁹, —C(O)OR⁹, —S(O)ₘR⁹, —N(R⁹)₂, —S(O)₂N(R⁹)₂, —NR⁹S(O)₂R⁹, —C(O)N(R⁹)₂ and —NR⁹C(O)R⁹.

Each R¹⁵ is independently selected from a halogen, —CN, —NO₂, oxo, a C₁₋₆ aliphatic radical, —R¹⁰, —C(O)R⁹, —C(O)OR⁹, —OR⁹, —S(O)ₘR⁹, —N(R⁹)₂, —S(O)₂N(R⁹)₂, —NR⁹S(O)₂R⁹, —C(O)N(R⁹)₂ and —NR⁹C(O)R⁹.

Each R^{15′} is independently selected from a halogen, —CN, —NO₂, a C₁₋₆ aliphatic radical, —R¹⁰, —C(O)R⁹, —C(O)OR⁹, —OR⁹, —S(O)ₘR⁹, —N(R⁹)₂, —S(O)₂N(R⁹)₂, —NR⁹S(O)₂R⁹, —C(O)N(R⁹)₂ and —NR⁹C(O)R⁹.

R¹⁶ and R¹⁷ are each independently selected from —H, deuterium, a C₁₋₆ alkyl, a C₁₋₆ haloalkyl and a halogen, or, alternatively, R¹⁶ and R¹⁷ are independently selected from a C₁₋₆ alkyl and a C₁₋₆ haloalkyl, and R¹⁶ and R¹⁷ taken together with the atom to which they are attached form a cyclopropyl or halocyclopropyl ring.

L is a linker selected from a methylene, —C(O)—, —O—, —S(O)ₘ— and —NR¹—;

wherein when L is a methylene, it is independently substituted with from 0 to 2 instances of R¹⁸.

m is 0, 1 or 2.

R¹ is selected from —H, a C₁₋₆ aliphatic radical, a C₃₋₆ cycloaliphatic, —CO(C₁₋₆ aliphatic), —CO(C₃₋₆ cycloaliphatic), —CO-(phenyl), a benzyl and —CO-(benzyl);

wherein when R¹ is selected from a C₁₋₆ aliphatic radical, —CO-(phenyl), a benzyl and —CO-(benzyl), it is independently substituted with from 0 to 3 instances of R^{B};

wherein each R^{B} is independently selected from a halogen, a C₁₋₂ alkyl and a C₁₋₂ alkoxy.

Each R¹⁸ is independently selected from a halogen, —CN, a C₁₋₆ aliphatic radical, a C₁₋₆ haloaliphatic radical, and a C₃₋₆ cycloaliphatic; or, alternatively, each R¹⁸ is independently selected from a C₁₋₆ aliphatic radical and a C₁₋₆ haloaliphatic radical, and two R¹⁸ groups, taken together with the atom to which they are attached, form a cyclopropyl or halocyclopropyl ring.

o is an integer selected from 0, 1 and 2.

Each J^{B} is independently selected from a halogen, —NO₂, —CN, —R¹⁹, —C(O)H, —C(O)OH, —C(O)NH₂, —OH, —SH, —NH₂, —C(O)R¹⁹, —C(O)OR¹⁹, —C(O)N(R²⁰)R¹⁹, —N(R²⁰)C(O)R¹⁹, —OR¹⁹, —SR¹⁹ and —NR¹⁹R²⁰; or, alternatively, two J^{B} groups are attached to two vicinal ring B atoms and, together with said ring atoms, form a 5 to 6-membered heterocycle or a 5 to 6-membered heteroaryl, each of said rings independently substituted with from 0 to 2 instances of R^{E}, wherein each R^{E} is independently selected from a halogen, a C₁₋₂ alkyl, a C₁₋₂ alkoxy, —CN and —OH.

Each R²⁰ is independently selected from a —H and a C₁₋₆ aliphatic radical.

Each R¹⁹ is independently selected from a C₁₋₆ aliphatic radical, a C₃₋₆ cycloaliphatic, a phenyl, a benzyl, a 4 to 6-membered heterocycle and a 5 to 6-membered heteroaryl; wherein: when R¹⁹ is a C₁₋₆ aliphatic radical, it is independently substituted with from 0 to 3 instances of R^{C}, wherein each R^{C} is independently selected from a halogen, —CN, —OH, —NH₂, a C₃₋₄ cycloalkyl, a C₃₋₄ halocycloalkyl, a —O(C₁₋₄ alkyl), a —O(C₃₋₄ cycloalkyl), a —O(C₃₋₄ halocycloalkyl), a —O(C₁₋₄ haloalkyl), a —NH(C₁₋₄ alkyl), a —N(C₁₋₄ alkyl)₂, and —NR^{V}; wherein —NR^{V} is a 4 to 6-membered heterocycle containing a ring N atom linked to J^{B}, and wherein said heterocycle contains from 0 to 2 additional ring heteroatoms selected from O and N; when R¹⁹ is a heterocycle or a heteroaryl it contains from 1 to 3 ring heteroatoms independently selected from N, O and S; when R¹⁹ is a phenyl, it is independently substituted with from 0 to 3 instances of R^{D}, wherein each R^{D} is independently selected from a halogen, a C₁₋₄ aliphatic radical, —CN, —OH, —NH₂, a —O(C₁₋₄ alkyl), a —NH(C₁₋₄ alkyl) and a —N(C₁₋₄ alkyl)₂; and when R¹⁹ is a non-aromatic ring or a heteroaryl, it is independently substituted with from 0 to 3 instances of R^{D′}, wherein each R^{D′} is independently selected from a halogen, oxo, a C₁₋₄ aliphatic radical, —CN, —OH, —NH₂, a —O(C₁₋₄ alkyl), a —NH(C₁₋₄ alkyl) and a —N(C₁₋₄ alkyl)₂.

L′ is a linker selected from —Y—SO₂—, —NR²¹SO₂—, —SO₂NR²¹—, —Y—C(O)—, —NR²¹C(O)— and —C(O)NR²¹—; wherein Y is selected from a single bond, a straight C₁₋₂ alkylene linker, and a branched C₂ alkylene linker, wherein the C₁₋₂ alkylene linker is independently substituted with from 0 to 3 halogen atoms.

R²¹ is selected from hydrogen, a C₁₋₆ alkyl, a C₁₋₆ haloalkyl, and a C₃₋₆ cycloalkyl ring.

n is an integer selected from 0, 1, 2 and 3.

Each J^{A} is independently selected from a halogen, —NO₂, —CN, —R²², —C(O)H, —C(O)OH, —C(O)NH₂, —OH, —SH and —NH₂, —C(O)R²², —C(O)OR²², —C(O)N(R²³)R²², —N(R²³)C(O)R²², —OR²², —SR²² and —NR²²R²³.

Each R²³ is selected from a —H and a C₁₋₆ aliphatic radical.

Each R²² is selected from a C₁₋₆ aliphatic radical, a C₃₋₆ cycloaliphatic ring, a phenyl, a benzyl, a 4 to 6-membered heterocycle and a 5 to 6-membered heteroaryl; wherein, when R²² is a C₁₋₆ aliphatic radical, it is independently substituted with from 0 to 3 instances of R^{F}, wherein each R^{F} is independently selected from a halogen, —CN, —OH, —NH₂, a C₃₋₄ cycloalkyl, a C₃₋₄ halocycloalkyl, a —O(C₁₋₄ alkyl), a —O(C₃₋₄ cycloalkyl), a —O(C₃₋₄ halocycloalkyl), a —O(C₁₋₄ haloalkyl), a —NH(C₁₋₄ alkyl), a —N(C₁₋₄ alkyl)₂ and —NR^{V}; wherein —NR^{V} is a 4 to 6-membered heterocycle containing a ring N atom linked to J^{B}, and wherein the heterocycle contains from 0 to 2 additional ring heteroatoms selected from O and N; when R²² is a heterocycle or a heteroaryl, the ring contains from 1 to 3 ring heteroatoms independently selected from N, O and S; when R²² is a non-aromatic ring or a 5 to 6-membered heteroaryl, it is independently substituted with from 0 to 3 instances of R^{G}, wherein each R^{G} is independently selected from a halogen, oxo, a C₁₋₄ aliphatic radical, —CN, —OH, —NH₂, a —O(C₁₋₄ alkyl), a —NH(C₁₋₄ alkyl) and a —N(C₁₋₄ alkyl)₂; and when R²² is a phenyl 1, it is independently substituted with from 0 to 3 instances of R^{G′}, wherein each R^{G′} is independently selected from a halogen, a C₁₋₄ aliphatic radical, —CN, —OH, —NH₂, —O(C₁₋₄ alkyl). —NH(C₁₋₄ alkyl) and —N(C₁₋₄ alkyl)₂.

In a second aspect, the present invention provides a method for preventing or lessening the severity of or treating a patient suffering from alopecia or acne comprising administering to said patient a therapeutically effective amount of a compound of Formula II, or a pharmaceutically acceptable salt thereof; or a pharmaceutical composition comprising the compound of Formula II, or a pharmaceutically acceptable salt thereof, and a pharmaceutically acceptable carrier; wherein the compound of Formula II is represented by the following structural formula:

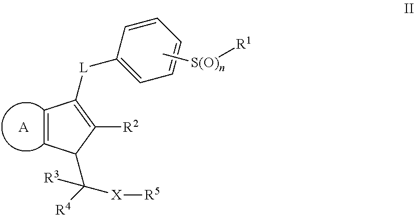

wherein
A is a 5, 6, 7, 8, 9 or 10-membered non-aromatic carbocycle; wherein A is optionally substituted with up to eight instances of R⁸;
L is chosen from
   —O—,
   —S(O)ₘ—,
   —NR¹⁴— and
   (C₁-C₃)alkylene, wherein, when said (C₁-C₃)alkylene is a C₂- or C₃-alkylene, one CH₂ is optionally replaced by —O—, —S(O)ₘ— or —NR¹⁴—, and wherein one or more substitutable carbon atoms of said (C₁-C₃)alkylene is optionally substituted with up to three instances of R¹¹;
X is chosen from a direct bond and (C₁-C₂)alkylene, wherein said (C₁-C₂)alkylene is optionally substituted with up to two instances of R¹²;
R¹ is chosen from (3-8-membered)carbocyclyl, (3-8-membered) heterocyclyl, —NR⁶(C₁-C₆)alkyl, —NR⁶(3-8-membered)carbocyclyl and —NR⁶(3-8-membered)heterocyclyl; wherein R¹ is optionally substituted with up to four instances of R⁹;
R² is chosen from hydrogen, halogen, (C₁-C₄)alkyl, (C₁-C₄)haloalkyl, and OH;
R³ is chosen from hydrogen, halogen, (C₁-C₄)alkyl and (C₁-C₄)haloalkyl;
R⁴ is chosen from hydrogen, halogen, (C₁-C₄)alkyl and (C₁-C₄)haloalkyl; or,
R³ and R⁴, taken together, form a (C₃-C₇)cycloalkyl ring;
R⁵ is chosen from C(O)OR⁷, C(O)N(R⁷)₂, C(O)NOR⁷ and C(O)NSR⁷;
R⁶ is chosen from H and (C₁-C₆)alkyl;
R⁷ is selected from hydrogen and (C₁-C₄)alkyl, wherein said (C₁-C₄)alkyl is optionally substituted with up to four instances of R¹⁶;
R⁸ in each occurrence is independently selected from halogen, (C₁-C₄)alkyl, (C₁-C₄)haloalkyl, (C₁-C₄)alkoxy, (C₁-C₄)haloalkoxy, CN, OH, oxo, and N(R¹⁴)₂;
R⁹ in each occurrence is independently selected from halogen, (C₁-C₄)alkyl, (C₁-C₄)haloalkyl, (C₁-C₄)alkylcarbonyl, (C₁-C₄)alkoxycarbonyl, CN, OH and N(R¹⁴)₂;
R¹⁰ in each occurrence is independently selected from halogen, (C₁-C₄)alkyl, (C₁-C₄)haloalkyl, (C₁-C₄)alkylcarbonyl, (C₁-C₄)alkoxycarbonyl, CN, OH and N(R¹⁵)₂;
R¹¹ in each occurrence is independently selected from halogen, (C₁-C₄)alkyl, (C₁-C₄)haloalkyl, (C₃-C₆)cycloalkyl, CN, OH, (C₁-C₄)alkoxy and (C₁-C₄)haloalkoxy;
R¹² in each occurrence is independently selected from halogen, (C₁-C₄)alkyl and (C₁-C₄)haloalkyl;
R¹⁴ is selected from hydrogen and (C₁-C₄)alkyl, wherein said (C₁-C₄)alkyl is optionally substituted with up to four instances of R¹⁰;
R¹⁵ is selected from hydrogen and (C₁-C₄)alkyl;
R¹⁶ in each occurrence is independently selected from halogen, (C₁-C₄)alkyl, (C₁-C₄)haloalkyl, (C₁-C₄)alkylcarbonyl, (C₁-C₄)alkoxycarbonyl, CN, OH and N(R¹⁵)₂;
m is zero, one or two; and
n is zero, one or two.

In a particular aspect of the methods disclosed herein, the compounds are administered in a daily or twice daily dose. The dose administered is typically between about 200 mg to about 1300 mg and may be given either orally or parentally.

### BRIEF DESCRIPTION OF THE DRAWINGS

FIG. 1 is a plot showing the plasma concentration of I-32 after 12 hours.

FIG. 2 is a plot showing the plasma concentration of I-32 after 24 hours.

### DETAILED DESCRIPTION

Reference will now be made in detail to certain embodiments of the invention, examples of which are illustrated in the accompanying structures and formulae. While the invention will be described in conjunction with the enumerated embodiments, it will be understood that they are not intended to limit the invention to those embodiments. Rather, the invention is intended to cover all alternatives, modifications and equivalents that may be included within the scope of the present invention as defined by the claims. The present invention is not limited to the methods and a material described herein but includes any methods and materials similar or equivalent to those described herein that could be used in the practice of the present invention. In the event that one or more of the incorporated literature references, patents or similar materials differ from or contradict this application, including but not limited to defined terms, term usage, described techniques or the like, this application controls.

### Description of Exemplary Compounds:

### DEFINITIONS AND GENERAL TERMINOLOGY

For purposes of this disclosure, the chemical elements are identified in accordance with the Periodic Table of the Elements, CAS version, and the Handbook of Chemistry and Physics, 75^{th} Ed. 1994. Additionally, general principles of organic chemistry are described in “Organic Chemistry”, Thomas Sorrell, University Science Books, Sausalito: 1999, and “March's Advanced Organic Chemistry”, 5^{th} Ed., Smith, M. B. and March, J., eds. John Wiley & Sons, New York: 2001, which are herein incorporated by reference in their entirety.

The compounds disclosed herein may optionally be substituted with one or more substituents, such as illustrated generally below, or as exemplified by particular classes, subclasses, and species of the invention. The phrase “optionally substituted” is used interchangeably with the phrase “substituted or unsubstituted.” In general, the term “substituted”, refers to the replacement of one or more hydrogen radicals in a given structure with the radical of a specified substituent. Unless otherwise indicated, an optionally substituted group may have a substituent at each substitutable position of the group. When more than one position in a given structure can be substituted with more than one substituent selected from a specified group, the substituent may be either the same or different at each position. If a substituent radical or structure is not identified or defined as “optionally substituted”, the substituent radical or structure is not substituted. As it will be apparent to one of ordinary skill in the art, groups such as —H, halogen, —NO₂, —CN, —OH, —NH₂ or —OCF₃ would not be substitutable groups.

The phrase “up to”, as used herein, refers to zero or any integer number that is equal or less than the number following the phrase. For example, “up to 3” means any one of 0, 1, 2, or 3. As described herein, a specified number range of atoms includes any integer therein. For example, a group having from 1-4 atoms could have 1, 2, 3 or 4 atoms. It will be understood by one of ordinary skill in the art that when a group is characterized as substituted (as opposed to optionally substituted) with, e.g., “up to 3” substituents, it can only be substituted with 1, 2 or 3 substituents.

When any variable occurs more than one time at any position, its definition on each occurrence is independent from every other occurrence, unless otherwise indicated.

Selection of substituents and combinations envisioned by this disclosure are only those that result in the formation of stable or chemically feasible compounds. Such choices and combinations will be apparent to those of ordinary sill in the art and may be determined without undue experimentation. The term “stable”, as used herein, refers to compounds that are not substantially altered when subjected to conditions to allow for their production, detection, and, in some embodiments, their recovery, purification, and use for one or more of the purposes disclosed herein. In some embodiments, a stable compound or chemically feasible compound is one that is not substantially altered when kept at a temperature of 25° C. or less, in the absence of moisture or other chemically reactive conditions, for at least a week.

A compound, such as the compounds herein disclosed, may be present in its free form (e.g. an amorphous form, a crystalline form or polymorphs). Under certain conditions, compounds may also form salts, and/or other multi-component crystalline forms (e.g. solvates, hydrates and co-crystals). As used herein, the term co-form is synonymous with the term multi-component crystalline form. When one of the components in the co-form has clearly transferred a proton to the other component, the resulting co-form is referred to as a “salt”. When both compounds in a multi-component crystalline form are independently solids at room temperature, the resulting co-form is referred to as a “co-crystal”. In co-crystals no proton transfer takes place between the different components of the co-form. The formation of a salt or a co-crystal is determined by how large is the difference in the pKas between the partners that form the mixture. As used herein, a “solvate” refers to an association or complex of one or more solvent molecules and a compound disclosed herein (or its salts or co-crystals). A “hydrate” is a particular type of solvate in which the solvent is water. Examples of solvents that can form solvates include, but are not limited to: water, isopropanol, ethanol, methanol, (dimethyl sulfoxide) DMSO, ethyl acetate, acetic acid, ethanolamine, tetrahydrofuran (THF), dichloromethane (DCM), N,N-dimethylformamide (DMF).

Unless only one of the isomers is drawn or named specifically, structures depicted herein are also meant to include all stereoisomeric (e.g., enantiomeric, diastereomeric, atropoisomeric and cis-trans isomeric) forms of the structure; for example, the R and S configurations for each asymmetric center, Ra and Sa configurations for each asymmetric axis, (Z) and (E) double bond configurations, and cis and trans conformational isomers. Therefore, single stereochemical isomers as well as racemates, and mixtures of enantiomers, diastereomers, and cis-trans isomers (double bond or conformational) of the present compounds are within the scope of the present disclosure. Unless otherwise stated, all tautomeric forms of the compounds of the present disclosure are within the scope of the disclosure.

The present disclosure also embraces isotopically-labeled compounds which are identical to those recited herein, but for the fact that one or more atoms are replaced by an atom having an atomic mass or mass number different from the atomic mass or mass number usually found in nature. All isotopes of any particular atom or element as specified are contemplated within the scope of the compounds disclosed herein, and their uses. Exemplary isotopes that can be incorporated into compounds disclosed herein include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I, and ¹²⁵I, respectively. Certain isotopically-labeled compounds of the present invention (e.g., those labeled with ³H and ¹⁴C) are useful in compound and/or substrate tissue distribution assays. Tritiated (i.e., ³H) and carbon-14 (i.e., ¹⁴C) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C, and ¹⁸F are useful for positron emission tomography (PET) studies to examine substrate receptor occupancy. Isotopically labeled compounds of the present invention can generally be prepared by following procedures analogous to those disclosed in the Schemes and/or in the Examples herein below, by substituting an isotopically labeled reagent for a non-isotopically labeled reagent.

The following definitions apply for the compounds of Formula I:

The term “aliphatic” or “aliphatic group” or “aliphatic radical”, as used herein, means a straight-chain (i.e., unbranched) or branched, substituted or unsubstituted hydrocarbon chain that is completely saturated or that contains one or more units of unsaturation. Unless otherwise specified, aliphatic groups contain 1-20 aliphatic carbon atoms. In some embodiments, aliphatic groups contain 1-10 aliphatic carbon atoms. In other embodiments, aliphatic groups contain 1-8 aliphatic carbon atoms. In still other embodiments, aliphatic groups contain 1-6 aliphatic carbon atoms. In other embodiments, aliphatic groups contain 1-4 aliphatic carbon atoms and in yet other embodiments, aliphatic groups contain 1-3 aliphatic carbon atoms. Suitable aliphatic groups include, but are not limited to, linear or branched, substituted or unsubstituted alkyl, alkenyl, or alkynyl groups. Specific examples of aliphatic groups include, but are not limited to: methyl, ethyl, propyl, butyl, isopropyl, isobutyl, vinyl, sec-butyl, tert-butyl, butenyl, propargyl, acetylene and the like.

The term “alkyl”, as used herein, refers to a saturated linear or branched-chain monovalent hydrocarbon radical. Unless otherwise specified, an alkyl group contains 1-20 carbon atoms (e.g., 1-20 carbon atoms, 1-10 carbon atoms, 1-8 carbon atoms, 1-6 carbon atoms, 1-4 carbon atoms or 1-3 carbon atoms). Examples of alkyl groups include, but are not limited to, methyl, ethyl, n-propyl, isopropyl, n-butyl, isobutyl, s-butyl, t-butyl, pentyl, hexyl, heptyl, octyl and the like.

The term “alkenyl” refers to a linear or branched-chain monovalent hydrocarbon radical with at least one site of unsaturation, i.e., a carbon-carbon, sp² double bond, wherein the alkenyl radical includes radicals having “cis” and “trans” orientations, or alternatively, “E” and “Z” orientations. Unless otherwise specified, an alkenyl group contains 2-20 carbon atoms (e.g., 2-20 carbon atoms, 2-10 carbon atoms, 2-8 carbon atoms, 2-6 carbon atoms, 2-4 carbon atoms or 2-3 carbon atoms). Examples include, but are not limited to, vinyl, allyl and the like.

The term “alkynyl” refers to a linear or branched monovalent hydrocarbon radical with at least one site of unsaturation, i.e., a carbon-carbon sp triple bond. Unless otherwise specified, an alkynyl group contains 2-20 carbon atoms (e.g., 2-20 carbon atoms, 2-10 carbon atoms, 2-8 carbon atoms, 2-6 carbon atoms, 2-4 carbon atoms or 2-3 carbon atoms). Examples include, but are not limited to, ethynyl, propynyl, and the like.

The term “carbocyclic” refers to a ring system formed only by carbon and hydrogen atoms. Unless otherwise specified, throughout this disclosure, carbocycle is used as a synonym of “non-aromatic carbocycle” or “cycloaliphatic”. In some instances the term can be used in the phrase “aromatic carbocycle”, and in this case it refers to an “aryl group” as defined below.

The term “cycloaliphatic” or “cycloaliphatic ring” (or “non-aromatic carbocycle”, “non-aromatic carbocyclyl”, “non-aromatic carbocyclic”) refers to a cyclic hydrocarbon that is completely saturated or that contains one or more units of unsaturation but which is not aromatic, and which has a single point of attachment to the rest of the molecule. Unless otherwise specified, a cycloaliphatic group may be monocyclic, bicyclic, tricyclic, fused, spiro or bridged. In one embodiment, the term “cycloaliphatic” refers to a monocyclic C₃-C₁₂ hydrocarbon or a bicyclic C₇-C₁₂ hydrocarbon. In some embodiments, any individual ring in a bicyclic or tricyclic ring system has 3 to 7 members. Suitable cycloaliphatic groups include, but are not limited to, cycloalkyl, cycloalkenyl, and cycloalkynyl. Examples of aliphatic groups include cyclopropyl, cyclobutyl, cyclopentyl, cyclopentenyl, cyclohexyl, cyclohexenyl, cycloheptyl, cycloheptenyl, norbornyl, cyclooctyl, cyclononyl, cyclodecyl, cycloundecyl, cyclododecyl, and the like.

The term “cycloaliphatic” also includes polycyclic ring systems in which the non-aromatic carbocyclic ring can be “fused” to one or more aromatic or non-aromatic carbocyclic or heterocyclic rings or combinations thereof, as long as the radical or point of attachment is on the non-aromatic carbocyclic ring.

“Heterocycle” (or “heterocyclyl” or “heterocyclic), as used herein, refers to a ring system in which one or more ring atoms are an independently selected heteroatom, which is completely saturated or that contains one or more units of unsaturation but which is not aromatic, and which has a single point of attachment to the rest of the molecule. Unless otherwise specified, through this disclosure, heterocycle is used as a synonym of “non-aromatic heterocycle”). In some instances the term can be used in the phrase “aromatic heterocycle”, and in this case it refers to a “heteroaryl group” as defined below. The term heterocycle also includes fused, spiro or bridged heterocyclic ring systems. Unless otherwise specified, a heterocycle may be monocyclic, bicyclic or tricyclic. In some embodiments, the heterocycle has 3 to 18 ring atoms in which one or more ring atoms is a heteroatom independently selected from oxygen, sulfur or nitrogen, and each ring in the system contains 3 to 7 ring atoms. In other embodiments, a heterocycle may be a monocycle having 3-7 ring atoms (2-6 carbon atoms and 1-4 heteroatoms) or a bicycle having 7-10 ring atoms (4-9 carbon atoms and 1-6 heteroatoms). Examples of bicyclic heterocyclic ring systems include, but are not limited to: adamantanyl, 2-oxa-bicyclo[2.2.2]octyl, 1-aza-bicyclo[2.2.2]octyl.

As used herein, the term “heterocycle” also includes polycyclic ring systems wherein the heterocyclic ring is fused with one or more aromatic or non-aromatic carbocyclic or heterocyclic rings, or with combinations thereof, as long as the radical or point of attachment is in the heterocyclic ring.

Examples of heterocyclic rings include, but are not limited to, the following monocycles: 2-tetrahydrofuranyl, 3-tetrahydrofuranyl, 2-tetrahydrothiophenyl, 3-tetrahydrothiophenyl, 2-morpholino, 3-morpholino, 4-morpholino, 2-thiomorpholino, 3-thiomorpholino, 4-thiomorpholino, 1-pyrrolidinyl, 2-pyrrolidinyl, 3-pyrrolidinyl, 1-tetrahydropiperazinyl, 2-tetrahydropiperazinyl, 3-tetrahydropiperazinyl, 1-piperidinyl, 2-piperidinyl, 3-piperidinyl, 1-pyrazolinyl, 3-pyrazolinyl, 4-pyrazolinyl, 5-pyrazolinyl, 1-piperidinyl, 2-piperidinyl, 3-piperidinyl, 4-piperidinyl, 2-thiazolidinyl, 3-thiazolidinyl, 4-thiazolidinyl, 1-imidazolidinyl, 2-imidazolidinyl, 4-imidazolidinyl, 5-imidazolidinyl; and the following bicycles: 3-1H-benzimidazol-2-one, 3-(1-alkyl)-benzimidazol-2-one, indolinyl, tetrahydroquinolinyl, tetrahydroisoquinolinyl, benzothiolane, benzodithiane, and 1,3-dihydro-imidazol-2-one.

As used herein, the term “aryl” (as in “aryl ring” or “aryl group”), used alone or as part of a larger moiety, as in “aralkyl”, “aralkoxy”, “aryloxyalkyl”, refers to a carbocyclic ring system wherein at least one ring in the system is aromatic and has a single point of attachment to the rest of the molecule. Unless otherwise specified, an aryl group may be monocyclic, bicyclic or tricyclic and contain 6-18 ring atoms. The term also includes polycyclic ring systems where the aryl ring is fused with one or more aromatic or non-aromatic carbocyclic or heterocyclic rings, or with combinations thereof, as long as the radical or point of attachment is in the aryl ring. Examples of aryl rings include, but are not limited to, phenyl, naphthyl, indanyl, indenyl, tetralin, fluorenyl, and anthracenyl.

The term “heteroaryl” (or “heteroaromatic” or “heteroaryl group” or “aromatic heterocycle”) used alone or as part of a larger moiety as in “heteroaralkyl” or “heteroarylalkoxy” refers to a ring system wherein at least one ring in the system is aromatic and contains one or more ring heteroatoms, wherein each ring in the system contains 3 to 7 ring atoms and which has a single point of attachment to the rest of the molecule. Unless otherwise specified, a heteroaryl ring system may be monocyclic, bicyclic or tricyclic and have a total of five to fourteen ring atoms. In one embodiment, all rings in a heteroaryl system are aromatic. Also included in this definition are heteroaryl radicals where the heteroaryl ring is fused with one or more aromatic or non-aromatic carbocyclic or heterocyclic rings, or combinations thereof, as long as the radical or point of attachment is in the heteroaryl ring. Bicyclic 6,5 heteroaromatic system, as used herein, for example, is a six membered heteroaromatic ring fused to a second five membered ring wherein the radical or point of attachment is on the six membered ring.

Heteroaryl rings include, but are not limited to the following monocycles: 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxazolyl, 4-oxazolyl, 5-oxazolyl, N-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, pyridazinyl (e.g., 3-pyridazinyl), 2-thiazolyl, 4-thiazolyl, 5-thiazolyl, tetrazolyl (e.g., 5-tetrazolyl), triazolyl (e.g., 2-triazolyl and 5-triazolyl), 2-thienyl, 3-thienyl, pyrazolyl (e.g., 2-pyrazolyl), isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, pyrazinyl, 1,3,5-triazinyl, and the following bicycles: benzimidazolyl, benzofuryl, benzothiophenyl, benzopyrazinyl, benzopyranonyl, indolyl (e.g., 2-indolyl), purinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl).

As used herein, “cyclo” (or “cyclic”, or “cyclic moiety”) encompasses mono-, bi- and tri-cyclic ring systems including cycloaliphatic, heterocyclic, aryl or heteroaryl, each of which has been previously defined.

“Fused” bicyclic ring systems comprise two rings which share two adjoining ring atoms.

“Bridged” bicyclic ring systems comprise two rings which share three or four adjacent ring atoms. As used herein, the term “bridge” refers to a bond or an atom or a chain of atoms connecting two different parts of a molecule. The two atoms that are connected through the bridge (usually but not always, two tertiary carbon atoms) are referred to as “bridgeheads”. Examples of bridged bicyclic ring systems include, but are not limited to, adamantanyl, norbornanyl, bicyclo[3.2.1]octyl, bicyclo[2.2.2]octyl, bicyclo[3.3.1]nonyl, bicyclo[3.2.3]nonyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.03,7]nonyl.

“Spiro” bicyclic ring systems share only one ring atom (usually, but not always, a quaternary carbon atom).

The term “ring atom” refers to an atom such as C, N, O or S that is part of the ring of an aromatic ring, a cycloaliphatic ring or a heteroaryl ring. A “substitutable ring atom” is a ring carbon or nitrogen atom bonded to at least one hydrogen atom. The hydrogen can be optionally replaced with a suitable substituent group. Thus, the term “substitutable ring atom” does not include ring nitrogen or carbon atoms which are shared when two rings are fused. In addition, “substitutable ring atom” does not include ring carbon or nitrogen atoms when the structure depicts that they are already attached to one or more moiety other than hydrogen and no hydrogens are available for substitution.

“Heteroatom” refers to one or more of oxygen, sulfur, nitrogen, phosphorus, or silicon, including any oxidized form of nitrogen, sulfur, phosphorus, or silicon, the quaternized form of any basic nitrogen, or a substitutable nitrogen of a heterocyclic or heteroaryl ring, for example N (as in 3,4-dihydro-2H-pyrrolyl), NH (as in pyrrolidinyl) or NR⁺ (as in N-substituted pyrrolidinyl).

In some embodiments, two independent occurrences of a variable may be taken together with the atom(s) to which each variable is bound to form a 5 to 8-membered, heterocyclyl, aryl, or heteroaryl ring or a 3 to 8-membered cycloalkyl ring. Exemplary rings that are formed when two independent occurrences of a substituent are taken together with the atom(s) to which each variable is bound include, but are not limited to the following: a) two independent occurrences of a substituent that are bound to the same atom and are taken together with that atom to form a ring, where both occurrences of the substituent are taken together with the atom to which they are bound to form a heterocyclyl, heteroaryl, carbocyclyl or aryl ring, wherein the group is attached to the rest of the molecule by a single point of attachment; and b) two independent occurrences of a substituent that are bound to different atoms and are taken together with both of those atoms to form a heterocyclyl, heteroaryl, carbocyclyl or aryl ring, wherein the ring that is formed has two points of attachment with the rest of the molecule. For example, where a phenyl group is substituted with two occurrences of —OR^{o} as in Formula D1:

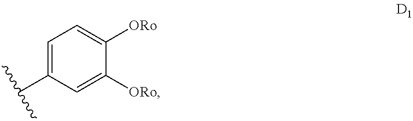

these two occurrences of —OR^{o} may be taken together with the aryl ring carbon atoms to which they are bound to form a fused 6-membered oxygen containing heterocyclic ring as in Formula D2:

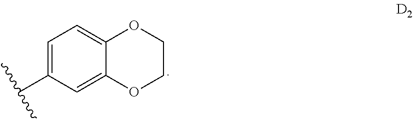 [SMILES: *c1ccc2c(c1)OCCO2]

It will be appreciated that a variety of other rings can be formed when two independent occurrences of a substituent are taken together with the atom(s) to which each substituent is bound and that the examples detailed above are not intended to be limiting.

In some embodiments, an alkyl or aliphatic chain can be optionally interrupted with another atom or group. This means that a methylene unit of the alkyl or aliphatic chain can optionally be replaced with said other atom or group. Unless otherwise specified, the optional replacements form a chemically stable compound. Optional interruptions can occur both within the chain and/or at either end of the chain; i.e. both at the point of attachment(s) to the rest of the molecule and/or at the terminal end. Two optional replacements can also be adjacent to each other within a chain so long as it results in a chemically stable compound. Unless otherwise specified, if the replacement or interruption occurs at a terminal end of the chain, the replacement atom is bound to a H on the terminal end. For example, if —CH₂CH₂CH₃ were optionally interrupted with —O—, the resulting chain could be —OCH₂CH₃, —CH₂OCH₃, or —CH₂CH₂OH. In another example, if the divalent linker —CH₂CH₂CH₂— were optionally interrupted with —O—, the resulting linker could be —OCH₂CH₂—, —CH₂OCH₂—, or —CH₂CH₂O—. The optional replacements can also completely replace all of the carbon atoms in a chain. For example, a C₃ aliphatic linker can be optionally replaced by —N(R^{$})—, —C(O)—, and —N(R^{$})— to form —N(R^{$})C(O)N(R^{$})— (a urea linker).

In general, the term “vicinal” refers to the placement of substituents on a group that includes two or more carbon atoms, wherein the substituents are attached to adjacent carbon atoms.

In general, the term “geminal” refers to the placement of substituents on a group that includes two or more carbon atoms, wherein the substituents are attached to the same carbon atom.

The terms “terminally” and “internally” refer to the location of a group within a substituent. A group is terminal when the group is present at the end of the substituent not further bonded to the rest of the chemical structure. Carboxyalkyl, i.e., R^{X}O(O)C-alkyl is an example of a carboxy group used terminally. A group is internal when the group is present in the middle of a substituent at the end of the substituent bound to the rest of the chemical structure. Alkylcarboxy (e.g., alkyl-C(O)O— or alkyl-O(CO)—) and alkylcarboxyaryl (e.g., alkyl-C(O)O-aryl- or alkyl-O(CO)-aryl-) are examples of carboxy groups used internally.

As described herein, a bond drawn from a substituent to the center of one ring within a multiple-ring system (as shown below), represents substitution with the substituent at any substitutable position in any of the rings within the multiple ring system. For example, formula D3 represents possible substitution with a substituent X at any of the positions shown in formula D4:

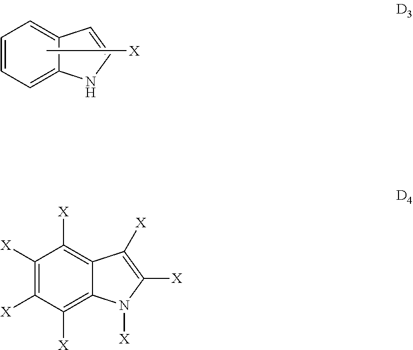

This also applies to multiple ring systems fused to optional ring systems (which would be represented by dotted lines). For example, in Formula D5, X is an optional substituent both for ring A and ring B (ring B being an optional ring).

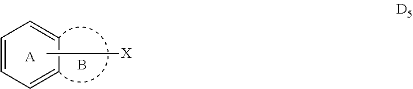

If, however, two rings in a multiple ring system each have different substituents drawn from the center of each ring, then, unless otherwise specified, each substituent only represents substitution on the ring to which it is attached. For example, in Formula D6, Y is an optional substituent for ring A only, and X is an optional substituent for ring B only.

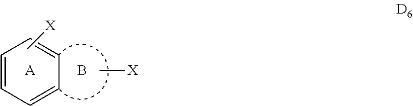

As used herein, the terms “alkoxy” or “alkylthio” refer to an alkyl group, as previously defined, attached to the molecule, or to another chain or ring, through an oxygen (“alkoxy” i.e, —O-alkyl) or a sulfur (“alkylthio” i.e., —S-alkyl) atom.

The terms Cₙ₋ₘ “alkoxyalkyl”, Cₙ₋ₘ “alkoxyalkenyl”, Cₙ₋ₘ “alkoxyaliphatic”, and Cₙ₋ₘ “alkoxyalkoxy” mean alkyl, alkenyl, aliphatic or alkoxy, as the case may be, substituted with one or more alkoxy groups, wherein the combined total number of carbons of the alkyl and alkoxy groups, alkenyl and alkoxy groups, aliphatic and alkoxy groups or alkoxy and alkoxy groups, combined, as the case may be, is between the values of n and m. For example, a C₄₋₆ alkoxyalkyl has a total of 4-6 carbons divided between the alkyl and alkoxy portion; e.g. it can be —CH₂OCH₂CH₂CH₃, —CH₂CH₂OCH₂CH₃ or —CH₂CH₂CH₂OCH₃.

When the moieties described in the preceding paragraph are optionally substituted, they can be substituted in either or both of the portions on either side of the oxygen or sulfur. For example, an optionally substituted C₄ alkoxyalkyl could be, for instance, —CH₂CH₂OCH₂(Me)CH₃ or —CH₂(OH)OCH₂CH₂CH₃; a C₅ alkoxyalkenyl could be, for instance, —CH═CHOCH₂CH₂CH₃ or —CH═CHCH₂OCH₂CH₃.

The terms aryloxy, arylthio, benzyloxy or benzylthio, refer to an aryl or benzyl group attached to the molecule, or to another chain or ring, through an oxygen (“aryloxy”, benzyloxy e.g., —O-Ph, —OCH₂Ph) or sulfur (“arylthio” e.g., —S-Ph, —S—CH₂Ph) atom. Further, the terms “aryloxyalkyl”, “benzyloxyalkyl” “aryloxyalkenyl” and “aryloxyaliphatic” mean alkyl, alkenyl or aliphatic, as the case may be, substituted with one or more aryloxy or benzyloxy groups, as the case may be. In this case, the number of atoms for each aryl, aryloxy, alkyl, alkenyl or aliphatic will be indicated separately. Thus, a 5-6-membered aryloxy(C₁₋₄alkyl) is a 5-6 membered aryl ring, attached via an oxygen atom to a C₁₋₄ alkyl chain which, in turn, is attached to the rest of the molecule via the terminal carbon of the C₁₋₄ alkyl chain.

As used herein, the terms “halogen” or “halo” mean F, Cl, Br, or I.

The terms “haloalkyl”, “haloalkenyl”, “haloaliphatic”, and “haloalkoxy” mean alkyl, alkenyl, aliphatic or alkoxy, as the case may be, substituted with one or more halogen atoms. For example a C₁₋₃ haloalkyl could be —CFHCH₂CHF₂ and a C₁₋₂ haloalkoxy could be —OC(Br)HCHF₂. This term includes perfluorinated alkyl groups, such as —CF₃ and —CF₂CF₃.

As used herein, the term “cyano” refers to —CN (or —C≡N).

The terms “cyanoalkyl”, “cyanoalkenyl”, “cyanoaliphatic”, and “cyanoalkoxy” mean alkyl, alkenyl, aliphatic or alkoxy, as the case may be, substituted with one or more cyano groups. For example a C₁₋₃ cyanoalkyl could be —C(CN)₂CH₂CH₃ and a C₁₋₂ cyanoalkenyl could be ═CHCH₂(CN).

As used herein, an “amino” group refers to —NH₂.

The terms “aminoalkyl”, “aminoalkenyl”, “aminoaliphatic”, and “aminoalkoxy” mean alkyl, alkenyl, aliphatic or alkoxy, as the case may be, substituted with one or more amino groups. For example a C₁₋₃ aminoalkyl could be —CH(NH₂)CH₂CH₂NH₂ and a C₁₋₂ aminoalkoxy could be —OCH₂CH₂NH₂.

The term “hydroxyl” or “hydroxy” refer to —OH.

The terms “hydroxyalkyl”, “hydroxyalkenyl”, “hydroxyaliphatic”, and “hydroxyalkoxy” mean alkyl, alkenyl, aliphatic or alkoxy, as the case may be, substituted with one or more —OH groups. For example a C₁₋₃ hydroxyalkyl could be —CH₂CH₂(OH)CH₃ and a C₄ hydroxyalkoxy could be —OCH₂C(CH₃)(OH)CH₃.

As used herein, a “carbonyl”, used alone or in connection with another group refers to —C(O)— or —C(O)H. For example, as used herein, an “alkoxycarbonyl,” refers to a group such as —C(O)O(alkyl).

As used herein, an “oxo” refers to ═O, wherein oxo is usually, but not always, attached to a carbon atom. An aliphatic chain can be optionally interrupted by a carbonyl group or can optionally be substituted by an oxo group, and both expressions refer to the same: e.g. —CH₂—C(O)—CH₃.

As used herein, in the context of resin chemistry (e.g. using solid resins or soluble resins or beads), the term “linker” refers to a bifunctional chemical moiety attaching a compound to a solid support or soluble support.

In all other situations, a “linker”, as used herein, refers to a divalent group in which the two free valences are on different atoms (e.g. carbon or heteroatom) or are on the same atom but can be substituted by two different substituents. For example, a methylene group can be C₁ alkyl linker (—CH₂—) which can be substituted by two different groups, one for each of the free valences (e.g. as in Ph-CH₂-Ph, wherein methylene acts as a linker between two phenyl rings). Ethylene can be C₂ alkyl linker (—CH₂CH₂—) wherein the two free valences are on different atoms. The amide group, for example, can act as a linker when placed in an internal position of a chain (e.g. —CONH—). A linker can be the result of interrupting an aliphatic chain by certain functional groups or of replacing methylene units on said chain by said functional groups. E.g. a linker can be a C₁₋₆ aliphatic chain in which up to two methylene units are substituted by —C(O)— or —NH— (as in —CH₂—NH—CH₂—C(O)—CH₂— or —CH₂—NH—C(O)—CH₂—). An alternative way to define the same —CH₂—NH—CH₂—C(O)—CH₂— and —CH₂—NH—C(O)—CH₂— groups is as a C₃ alkyl chain optionally interrupted by up to two —C(O)— or —NH— moieties. Cyclic groups can also form linkers: e.g. a 1,6-cyclohexanediyl can be a linker between two R groups, as in

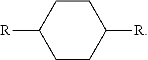

A linker can additionally be optionally substituted in any portion or position.

Divalent groups of the type R—CH═ or R₂C═, wherein both free valences are in the same atom and are attached to the same substituent, are also possible. In this case, they will be referred to by their IUPAC accepted names. For instance an alkylidene (such as, for example, a methylidene (═CH₂) or an ethylidene (═CH—CH₃)) would not be encompassed by the definition of a linker in this disclosure.

The term “protecting group”, as used herein, refers to an agent used to temporarily block one or more desired reactive sites in a multifunctional compound. In certain embodiments, a protecting group has one or more, or preferably all, of the following characteristics: a) reacts selectively in good yield to give a protected substrate that is stable to the reactions occurring at one or more of the other reactive sites; and b) is selectively removable in good yield by reagents that do not attack the regenerated functional group. Exemplary protecting groups are detailed in Greene, T. W., Wuts, P. G in “Protective Groups in Organic Synthesis”, Third Edition, John Wiley & Sons, New York: 1999, the entire contents of which are hereby incorporated by reference. The term “nitrogen protecting group”, as used herein, refers to an agents used to temporarily block one or more desired nitrogen reactive sites in a multifunctional compound. Preferred nitrogen protecting groups also possess the characteristics exemplified above, and certain exemplary nitrogen protecting groups are also detailed in Chapter 7 in Greene, T. W., Wuts, P. G in “Protective Groups in Organic Synthesis”, Third Edition, John Wiley & Sons, New York: 1999. As used herein, the term “displaceable moiety” or “leaving group” refers to a group that is associated with an aliphatic or aromatic group as defined herein and is subject to being displaced by nucleophilic attack by a nucleophile.

As used herein, “amide coupling agent” or “amide coupling reagent” means a compound that reacts with the hydroxyl moiety of a carboxy moiety thereby rendering it susceptible to nucleophilic attack. Exemplary amide coupling agents include DIC (diisopropylcarbodiimide), EDCI (1-Ethyl-3-(3-dimethylaminopropyl)carbodiimide), DCC (dicyclohexylcarbodiimide), BOP (Benzotriazol-1-yloxy-tris(dimethylamino)-phosphonium hexafluorophosphate), pyBOP ((Benzotriazol-1-yloxy)tripyrrolidinophosphonium Hexafluorophosphate), 2,4,6-tripropyl-1,3,5,2,4,6-trioxatriphosphorinane-2,4,6-trioxide (T₃P), etc.

The following definitions apply for the compounds of Formula II:

Hydrocarbon refers to any substituent comprised of hydrogen and carbon as the only elemental constituents. C₁ to C₂₀ hydrocarbon includes alkyl, cycloalkyl, polycycloalkyl, alkenyl, alkynyl, aryl and combinations thereof. Examples of C₁ to C₂₀ hydrocarbon include benzyl, phenethyl, cyclohexylmethyl, camphoryl and naphthylethyl. Unless otherwise specified, alkenyl is intended to include linear chain, branched chain or cyclic unsaturated hydrocarbon groups have at least carbon to carbon double bond, but no carbon to carbon triple bonds. Unless otherwise specified, alkynyl is intended to include linear chain, branched chain or cyclic unsaturated hydrocarbon groups have at least one carbon to carbon triple bond, wherein the alkynyl optionally can have one or more carbon to carbon double bonds.

Unless otherwise specified, the term “carbocycle” is intended to include ring systems in which the ring atoms are all carbon but of any oxidation state. Thus (C₃-C₁₀) carbocycle refers to both non-aromatic and aromatic systems, including such systems as cyclopropane, benzene and cyclohexene; (C₈-C₁₂) carbopolycycle refers to such systems as norbornane, decalin, indane and naphthalene. Carbocycle, if not otherwise limited, refers to monocycles, bicycles and polycycles.

Alkoxy or alkoxyl refers to groups of from 1 to 8 carbon atoms of a straight, branched, cyclic configuration and combinations thereof attached to the parent structure through an oxygen. Examples of alkoxy or alkoxyl include methoxy, ethoxy, propoxy, isopropoxy, cyclopropyloxy, cyclohexyloxy and the like. Loweralkoxy refers to groups containing one to four carbons. For the purpose of this application, alkoxy and lower alkoxy include methylenedioxy and ethylenedioxy.

Oxaalkyl refers to alkyl residues in which one or more carbons (and their associated hydrogens) have been replaced by oxygen. Examples include methoxypropoxy, 3,6,9-trioxadecyl and the like. The term oxaalkyl is intended as it is understood in the art [see Naming and Indexing of Chemical Substances for Chemical Abstracts, published by the American Chemical Society, 196, but without the restriction of 127(a)], i.e. it refers to compounds in which the oxygen is bonded via a single bond to its adjacent atoms (forming ether bonds); it does not refer to doubly bonded oxygen, as would be found in carbonyl groups. Similarly, thiaalkyl and azaalkyl refer to alkyl residues in which one or more carbons have been replaced by sulfur or nitrogen, respectively. Examples include ethylaminoethyl and methylthiopropyl.

Unless otherwise specified, acyl refers to formyl and to groups of 1, 2, 3, 4, 5, 6, 7 or 8 carbon atoms of a straight chain, branched chain, cyclic configuration, saturated, unsaturated and aromatic and combinations thereof, attached to the parent structure through a carbonyl functionality. One or more carbons in the acyl residue may be replaced by nitrogen, oxygen or sulfur as long as the point of attachment to the parent remains at the carbonyl. Examples include acetyl, benzoyl, propionyl, isobutyryl, t-butoxycarbonyl, benzyloxycarbonyl and the like. Lower-acyl refers to groups containing one to four carbons. The double bonded oxygen, when referred to as a substituent itself is called “oxo”.

Aryl means (i) a monocyclic 6-membered aromatic ring; (ii) a bicyclic 9- or 10-membered aromatic ring system; or (iii) a tricyclic 13- or 14-membered aromatic or ring system. Heteroaryl mean (i) a monocyclic 5- or 6-membered heteroaromatic ring containing 1-3 heteroatoms selected from O, N, or S or a phenyl group (or benzene); (ii) a bicyclic 9- or 10-membered aromatic or heteroaromatic ring system containing 1-4 heteroatoms selected from O, N, or S; or (iii) a tricyclic 13- or 14-membered aromatic or heteroaromatic ring system containing 1-5 heteroatoms selected from O, N, or S. The aromatic 6- to 14-membered carbocyclic rings include, e.g., benzene, naphthalene, indane, tetralin, and fluorene. 5- to 10-membered aromatic heterocyclic rings include, e.g., imidazole, pyridine, indole, thiophene, benzopyranone, thiazole, furan, benzimidazole, quinoline, isoquinoline, quinoxaline, pyrimidine, pyrazine, tetrazole and pyrazole.

Arylalkyl refers to a substituent in which an aryl residue is attached to the parent structure through alkyl. Examples of arylalkyl are benzyl, phenethyl and the like. Heteroarylalkyl refers to a substituent in which a heteroaryl residue is attached to the parent structure through alkyl. In one embodiment, the alkyl group of an arylalkyl or a heteroarylalkyl is an alkyl group of from 1 to 6 carbons. Examples of heteroarylalkyl include, e.g., pyridinylmethyl, pyrimidinylethyl and the like.

Heterocycle means a cycloalkyl or aryl carbocycle residue in which from one to three carbons is replaced by a heteroatom selected from the group consisting of N, O and S. The nitrogen and sulfur heteroatoms may optionally be oxidized, and the nitrogen heteroatom may optionally be quaternized. Unless otherwise specified, a heterocycle may be non-aromatic or aromatic. Examples of heterocycles that fall within the scope of the invention include pyrrolidine, pyrazole, pyrrole, indole, quinoline, isoquinoline, tetrahydroisoquinoline, benzofuran, benzodioxan, benzodioxole (commonly referred to as methylenedioxyphenyl, when occurring as a substituent), tetrazole, morpholine, thiazole, pyridine, pyridazine, pyrimidine, thiophene, furan, oxazole, oxazoline, isoxazole, dioxane, tetrahydrofuran and the like. It is to be noted that heteroaryl is a subset of heterocycle in which the heterocycle is aromatic. Examples of heterocyclyl residues additionally include piperazinyl, 2-oxopiperazinyl, 2-oxopiperidinyl, 2-oxo-pyrrolidinyl, 2-oxoazepinyl, azepinyl, 4-piperidinyl, pyrazolidinyl, imidazolyl, imidazolinyl, imidazolidinyl, pyrazinyl, oxazolidinyl, isoxazolidinyl, thiazolidinyl, isothiazolyl, quinuclidinyl, isothiazolidinyl, benzimidazolyl, thiadiazolyl, benzopyranyl, benzothiazolyl, tetrahydrofuryl, tetrahydropyranyl, thienyl, benzothienyl, thiamorpholinyl, thiamorpholinylsulfoxide, thiamorpholinylsulfone, oxadiazolyl, triazolyl and tetrahydroquinolinyl.

The term “substituted” refers to the replacement of one or more hydrogen atoms in a specified group with a specified radical. For example, substituted alkyl, aryl, cycloalkyl, heterocyclyl, etc., refer to alkyl, aryl, cycloalkyl, or heterocyclyl wherein one or more H atoms in each alkyl, aryl, cycloalkyl or heterocyclyl residue are replaced with halogen, haloalkyl, alkyl, acyl, alkoxyalkyl, hydroxyloweralkyl, phenyl, heteroaryl, benzenesulfonyl, hydroxy, loweralkoxy, haloalkoxy, carboxy, alkoxycarbonyl [—C(═O)O-alkyl], alkoxycarbonylamino [HNC(═O)O-alkyl], carboxamido [—C(═O)NH₂], alkylaminocarbonyl [—C(═O)NH-alkyl], cyano, acetoxy, nitro, amino, alkylamino, dialkylamino, mercapto, alkylthio, sulfoxide, sulfone, sulfonylamino, acylamino, amidino, aryl, benzyl, heterocyclyl, phenoxy, benzyloxy, heteroaryloxy, hydroxyimino, alkoxyimino, oxaalkyl, aminosulfonyl, trityl, amidino, guanidino, ureido, and benzyloxy. “Oxo” is also included among the substituents referred to in “optionally substituted”; it will be appreciated by persons of skill in the art that, because oxo is a divalent radical, there are circumstances in which it will not be appropriate as a substituent (e.g. on phenyl). In one embodiment, 1, 2 or 3 hydrogen atoms of any one of the alkyl, aryl, cycloalkyl and heterocyclyl residues are replaced with the above specified substituents.

The terms “haloalkyl” and “haloalkoxy” mean alkyl or alkoxy, respectively, substituted with one or more halogen atoms. The terms “alkylcarbonyl” and “alkoxycarbonyl” mean —C(═O)alkyl or —C(O)alkoxy, respectively.

The term “halogen” means fluorine, chlorine, bromine or iodine. In one embodiment, halogen may be fluorine or chlorine.

Substituents IV are generally defined when introduced and retain that definition throughout the specification and in all independent claims.

When used in a structural or chemical formula, “Et” refers to an ethyl group (—CH₂CH₃), “Me” refers to a methyl group (—CH₃), and “Ph” refers to a phenyl group (—C₆H₆).

The compounds disclosed herein are defined herein by their chemical structures and/or chemical names. Where a compound is referred to by both a chemical structure and a chemical name, and the chemical structure and chemical name conflict, the chemical structure is determinative of the compound's identity.

### Methods of Use

In another aspect, the present invention also provides a method for preventing or lessening the severity of or treating a patient suffering from alopecia or acne in a patient comprising administering to said patient a therapeutically effective amount of a compound of the Formula I or Formula II.

In one embodiment of this aspect, the patient is suffering from alopecia. Alopecia includes androgenetic alopecia, toxic alopecia, alopecia greata, trichotillomania or scarring alopecia. In particular embodiment, the alopecia is androgenetic alopecia.

Alopecia is characterized by hair loss and may affect any part of the subject or patient's body. Hair loss on the head is commonly known as “baldness”. Alopecia may develop gradually or suddenly and may be the result of hereditary factors, aging, local skin conditions, disease, or drug use/treatment. Androgenetic alopecia is the most common type of hair loss and affecting both men “male pattern baldness” and women “female pattern baldness”. Toxic alopecia results from physical or psychological stress, including, for example, severve illness, sudden weight loss, sugery, or drug treatment (such a chemotherapy drugs, blood pressure drugs, lithium, valproate, oral contraceptives, vitamin A and retinoids), underactive thyroid gland or pituary gland and can be common after pregnancy. Alopecia greata is characterized by the loss of round, irregular patches of hair, often on the scalp or beard, but all body hair may also be lost (alopecia universalis), and may be caused by an autoimmune reaction. Scarring alopecia is hair loss that occurs at scarred or damages areas.

In one embodiment of this aspect, the patient is suffering from acne.

Acne occurs mostly on the face, upper chest, shoulders and back. Sebaceous glands, which secrete sebum, are attached to hair follicles. The sebum, along with the dead skin cells, passes up from the sebaceous gland and hair follicle and out to the surface of the skin through the pores. Acne results when a collection of dried sebum, dead skin cells, and/or bacteria clog the hair follicles and the sebum is prevented from leaving the pore. Acne can be mild to very severe. Acne Vulgaris (most common form of acne) and Acne rosacea is arew two forms of acne. Mild to moderate acne vulgaris consists of blackheads, whiteheads, papules, pustules. Severe acne vulgaris includes nodules and cysts. Cystic acne is an example of severe acne.

In one aspect, the present invention provides a method for preventing or lessening the severity of or treating a patient suffering from alopecia or acne comprising administering to said patient a therapeutically effective amount of a compound of Formula I, or a pharmaceutically acceptable salt thereof; or a pharmaceutical composition comprising the compound of Formula I, or a pharmaceutically acceptable salt thereof, and a pharmaceutically acceptable carrier; wherein the compound of Formula I is represented by the following structural formula:

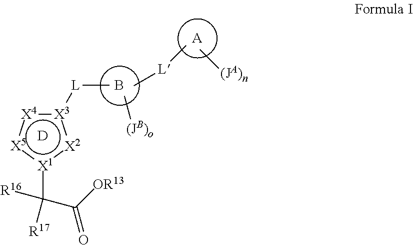

wherein:

ring A is a monocyclic or bicyclic ring selected from a 6 to 10-membered aryl, a 5 to 10-membered heteroaryl, a C₃₋₁₀ cycloaliphatic and a 4 to 10-membered heterocycle; wherein said heteroaryl or heterocycle contains from 0 to 3 ring heteroatoms independently selected from N, O and S;

ring B is a monocyclic ring selected from a phenyl and a 5 to 6-membered heteroaryl, wherein said heteroaryl contains up to three ring heteroatoms independently selected from N, O and S;

ring D is a 5-membered heteroaryl; wherein

x¹ is selected from N and C;

x² is selected from N and C—R²;

x³ is selected from N and C;

x⁴ is selected from N and C—R⁴; and

x⁵ is selected from N and C—R⁵;

provided that at least one of x¹ or x³ is N, but both are not simultaneously N;

R² is selected from —H, a halogen, —NO₂, —CN, a C₁₋₆ aliphatic radical, a C₁₋₆ alkoxy and a cyclopropyl ring, wherein R² is independently substituted with from 0 to 3 instances of R^{A}; wherein

each R^{A} is independently selected from a halogen, —OH, a C₁₋₂ alkoxy and a C₁₋₂ haloalkoxy;

R⁴ is selected from a halogen, —NO₂, —CN, —R⁶, —OR⁶, —C(O)R⁶, —C(O)OR⁶, —N(R⁶)₂, —S(O)ₚR⁶, —S(O)₂N(R⁶)₂, —NR⁶S(O)₂R⁶, —C(O)N(R⁶)₂ and —NR⁶C(O)R⁶;

R⁵ is selected from a halogen, —NO₂, —CN, —R⁶, —OR⁶, —C(O)R⁶, —C(O)OR⁶, —N(R⁶)₂, —S(O)ₚR⁶, —S(O)₂N(R⁶)₂, —NR⁶S(O)₂R⁶, —C(O)N(R⁶)₂ and —NR⁶C(O)R⁶;

p is an integer selected from 0, 1 and 2;

each R⁶ is independently selected from —H, a C₁₋₆ aliphatic radical, and a monocyclic or bicyclic ring; wherein

the ring is selected from a 6 to 10-membered aryl, a 5 to 10-membered heteroaryl, a C₃₋₁₀ cycloaliphatic and a 4 to 10-membered heterocycle; wherein

when R⁶ is a C₁₋₆ aliphatic radical, it is independently substituted with from 0 to 6 instances of R⁷,

when R⁶ is a non-aromatic ring or a heteroaryl, it is independently substituted with from 0 to 6 instances of R⁸, and

when R⁶ is an aryl, it is independently substituted with from 0 to 6 instances of R^{8′};

each R⁷ is independently selected from a halogen, —CN, oxo, —OR⁹, —R¹⁰, —C(O)R⁹, —C(O)OR⁹, —S(O)ₘR⁹, —N(R⁹)₂, —S(O)₂N(R⁹)₂, —NR⁹S(O)₂R⁹, —C(O)N(R⁹)₂ and —NR⁹C(O)R⁹;

each R⁸ is independently selected from a halogen, —CN, —NO₂, oxo, a C₁₋₆ aliphatic radical, —R¹⁰, —C(O)R⁹, —C(O)OR⁹, —OR⁹, —S(O)ₘR⁹, —N(R⁹)₂, —S(O)₂N(R⁹)₂, —NR⁹S(O)₂R⁹, —C(O)N(R⁹)₂ and —NR⁹C(O)R⁹;

each R⁸ is independently selected from a halogen, —CN, —NO₂, a C₁₋₆ aliphatic radical, —R¹⁰, —C(O)R⁹, —C(O)OR⁹, —OR⁹, —S(O)ₘR⁹, —N(R⁹)₂, —S(O)₂N(R⁹)₂, —NR⁹S(O)₂R⁹, —C(O)N(R⁹)₂ and —NR⁹C(O)R⁹;

each R⁹ is independently selected from hydrogen, a C₁₋₆ aliphatic radical and a monocyclic or bicyclic ring, wherein

the ring is selected from a 6 to 10-membered aryl, a 5 to 10-membered heteroaryl, a C₃₋₁₀ cycloaliphatic and a 4 to 10-membered heterocycle; wherein,

when R⁹ is a C₁₋₆ aliphatic radical, it is independently substituted with from 0 to 6 instances of R¹¹, and

when R⁹ is a ring, it is independently substituted with from 0 to 3 instances of R¹²;

each R¹⁰ is a monocyclic or bicyclic ring independently selected from a 6 to 10-membered aryl, a 5 to 10-membered heteroaryl, a C₃₋₁₀ cycloaliphatic and a 4 to 10-membered heterocycle; wherein

each R¹⁰ is independently substituted with from 0 to 3 instances of R¹²;

each R¹¹ is independently selected from a halogen, —CN, —OH, a C₁₋₄ alkoxy and a C₁₋₄ haloalkoxy;

each R¹² is independently selected from a halogen, —CN, —OH, a C₁₋₄ alkyl, a C₁₋₄ haloalkyl, a C₁₋₄ alkoxy and a C₁₋₄ haloalkoxy;

R¹³ is selected from —H, a C₁₋₆ aliphatic radical, and a monocyclic or bicyclic ring;

wherein the ring is selected from a 6 to 10-membered aryl, a 5 to 10-membered heteroaryl, a C₃₋₁₀ cycloaliphatic and a 4 to 10-membered heterocycle; wherein

when R¹³ is a C₁₋₆ aliphatic radical, it is independently substituted with from 0 to 6 instances of R¹⁴;

when R¹³ is a non-aromatic ring or a heteroaryl, it is independently substituted with from 0 to 6 instances of R¹⁵, and

when R¹³ is an aryl, it is independently substituted with from 0 to 6 instances of R^{15′},

each R¹⁴ is independently selected from a halogen, —CN, oxo, —OR⁹, —R¹⁰, —C(O)R⁹, —C(O)OR⁹, —S(O)ₘR⁹, —N(R⁹)₂, —S(O)₂N(R⁹)₂, —NR⁹S(O)₂R⁹, —C(O)N(R⁹)₂ and —NR⁹C(O)R⁹;

each R¹⁵ is independently selected from a halogen, —CN, —NO₂, oxo, a C₁₋₆ aliphatic radical, —R¹⁰, —C(O)R⁹, —C(O)OR⁹, —OR⁹, —S(O)ₘR⁹, —N(R⁹)₂, —S(O)₂N(R⁹)₂, —NR⁹S(O)₂R⁹, —C(O)N(R⁹)₂ and —NR⁹C(O)R⁹; and

each R^{15′} is independently selected from a halogen, —CN, —NO₂, a C₁₋₆ aliphatic radical, —R¹⁰, —C(O)R⁹, —C(O)OR⁹, —OR⁹, —S(O)ₘR⁹, —N(R⁹)₂, —S(O)₂N(R⁹)₂, —NR⁹S(O)₂R⁹, —C(O)N(R⁹)₂ and —NR⁹C(O)R⁹;

R¹⁶ and R¹⁷ are each independently selected from —H, deuterium, a C₁₋₆ alkyl, a C₁₋₆ haloalkyl and a halogen, or

alternatively, R¹⁶ and R¹⁷ are independently selected from a C₁₋₆ alkyl and a C₁₋₆ haloalkyl, and R¹⁶ and R¹⁷ taken together with the atom to which they are attached form a cyclopropyl or halocyclopropyl ring;

L is a linker selected from a methylene, —C(O)—, —O—, —S(O)ₘ— and —NR¹—; wherein

when L is a methylene, it is independently substituted with from 0 to 2 instances of R¹⁸;

m is 0, 1 or 2;

R¹ is selected from —H, a C₁₋₆ aliphatic radical, a C₃₋₆ cycloaliphatic, —CO(C₁₋₆ aliphatic), —CO(C₃₋₆ cycloaliphatic), —CO-(phenyl), a benzyl and —CO-(benzyl); wherein

when R¹ is selected from a C₁₋₆ aliphatic radical, —CO-(phenyl), a benzyl and —CO-(benzyl), it is independently substituted with from 0 to 3 instances of R^{B}; wherein

each R^{B} is independently selected from a halogen, a C₁₋₂ alkyl and a C₁₋₂ alkoxy;

each R¹⁸ is independently selected from a halogen, —CN, a C₁₋₆ aliphatic radical, a C₁₋₆ haloaliphatic radical, and a C₃₋₆ cycloaliphatic; or

alternatively, each R¹⁸ is independently selected from a C₁₋₆ aliphatic radical and a C₁₋₆ haloaliphatic radical, and two R¹⁸ groups, taken together with the atom to which they are attached form a cyclopropyl or a halocyclopropyl ring;

o is an integer selected from 0, 1 and 2;

each J^{B} is independently selected from a halogen, —NO₂, —CN, —R¹⁹, —C(O)H, —C(O)OH, —C(O)NH₂, —OH, —SH, —NH₂, —C(O)R¹⁹, —C(O)OR¹⁹, —C(O)N(R²⁰)R¹⁹, —N(R²⁰)C(O)R¹⁹, —OR¹⁹, —SR¹⁹ and —NR¹⁹R²⁰; or

alternatively, two J^{B} groups are attached to two vicinal ring B atoms and, together with said ring atoms, form a 5 to 6-membered heterocycle or a 5 to 6-membered heteroaryl, each of said rings independently substituted with from 0 to 2 instances of R^{E}, wherein each R^{E} is independently selected from a halogen, a C₁₋₂ alkyl, a C₁₋₂ alkoxy, —CN and —OH;

each R²⁰ is independently selected from a —H and a C₁₋₆ aliphatic radical;

each R¹⁹ is independently selected from a C₁₋₆ aliphatic radical, a C₃₋₆ cycloaliphatic, a phenyl, a benzyl, a 4 to 6-membered heterocycle and a 5 to 6-membered heteroaryl; wherein

when R¹⁹ is a C₁₋₆ aliphatic radical, it is independently substituted with from 0 to 3 instances of R^{C}, wherein each R^{C} is independently selected from a halogen, —CN, —OH, —NH₂, a C₃₋₄ cycloalkyl, a C₃₋₄ halocycloalkyl, a —O(C₁₋₄ alkyl), a —O(C₃₋₄ cycloalkyl), a —O(C₃₋₄ halocycloalkyl), a —O(C₁₋₄ haloalkyl), a —NH(C₁₋₄ alkyl), a —N(C₁₋₄ alkyl)₂, and —NR^{V}; wherein

—NR^{V} is a 4 to 6-membered heterocycle containing a ring N atom linked to J^{B}, and wherein said heterocycle contains from 0 to 2 additional ring heteroatoms selected from O and N;

when R¹⁹ is a heterocycle or a heteroaryl it contains from 1 to 3 ring heteroatoms independently selected from N, O and S;

when R¹⁹ is a phenyl, it is independently substituted with from 0 to 3 instances of R^{D}, wherein each R^{D} is independently selected from a halogen, a C₁₋₄ aliphatic radical, —CN, —OH, —NH₂, a —O(C₁₋₄ alkyl), a —NH(C₁₋₄ alkyl) and a —N(C₁₋₄ alkyl)₂; and

when R¹⁹ is a non-aromatic ring or a heteroaryl, it is independently substituted with from 0 to 3 instances of R^{D′}, wherein each R^{D′} is independently selected from a halogen, oxo, a C₁₋₄ aliphatic radical, —CN, —OH, —NH₂, a —O(C₁₋₄ alkyl), a —NH(C₁₋₄ alkyl) and a —N(C₁₋₄ alkyl)₂;

L′ is a linker selected from —Y—SO₂—, —NR²¹SO₂—, —SO₂NR²¹—, —Y—C(O)—, —NR²¹C(O)— and —C(O)NR²¹—; wherein

Y is selected from a single bond, a straight C₁₋₂ alkylene linker, and a branched C₂ alkylene linker, wherein the C₁₋₂ alkylene linker is independently substituted with from 0 to 3 a halogen atoms;

R²¹ is selected from hydrogen, a C₁₋₆ alkyl, a C₁₋₆ haloalkyl and a C₃₋₆ cycloalkyl ring;

n is an integer selected from 0, 1, 2 and 3;

each J^{A} is independently selected from a halogen, —NO₂, —CN, —R²², —C(O)H, —C(O)OH, —C(O)NH₂, —OH, —SH and —NH₂, —C(O)R²², —C(O)OR²², —C(O)N(R²³)R²², —N(R²³)C(O)R²², —OR²², —SR²² and —NR²²R²³;

each R²³ is independently selected from a —H and a C₁₋₆ aliphatic radical;

each R²² is independently selected from a C₁₋₆ aliphatic radical, a C₃₋₆ cycloaliphatic ring, a phenyl, a benzyl, a 4 to 6-membered heterocycle and a 5 to 6-membered heteroaryl; wherein

when R²² is a C₁₋₆ aliphatic radical, it is independently substituted with from 0 to 3 instances of R^{F}, wherein each R^{F} is independently selected from a halogen, —CN, —OH, —NH₂, a C₃₋₄ cycloalkyl, a C₃₋₄ halocycloalkyl, a —O(C₁₋₄ alkyl), a —O(C₃₋₄ cycloalkyl), a —O(C₃₋₄ halocycloalkyl), a —O(C₁₋₄ haloalkyl), a —NH(C₁₋₄ alkyl), a —N(C₁₋₄ alkyl)₂ and —NR^{V}; wherein

—NR^{V} is a 4 to 6-membered heterocycle containing a ring N atom linked to J^{B}, and wherein the heterocycle contains from 0 to 2 additional ring heteroatoms selected from O and N;

when R²² is a heterocycle or a heteroaryl, the ring contains from 1 to 3 ring heteroatoms independently selected from N, O and S;

when R²² is a non-aromatic ring or a 5 to 6-membered heteroaryl, it is independently substituted with from 0 to 3 instances of R^{G}, wherein

each R^{G} is independently selected from a halogen, oxo, a C₁₋₄ aliphatic radical, —CN, —OH, —NH₂, a —O(C₁₋₄ alkyl), a —NH(C₁₋₄ alkyl) and a —N(C₁₋₄ alkyl)₂; and

when R²² is a phenyl 1, it is independently substituted with from 0 to 3 instances of R^{G′}, wherein

each R^{G′} is independently selected from a halogen, a C₁₋₄ aliphatic radical, —CN, —OH, —NH₂, —O(C₁₋₄ alkyl), —NH(C₁₋₄ alkyl) and —N(C₁₋₄ alkyl)₂.

In some embodiments, ring A is selected from a phenyl, a 5 to 6-membered heteroaryl, a C₃₋₆ cycloaliphatic or a 5 to 6-membered heterocycle, wherein said heteroaryl or heterocycle contains from 1 to 2 ring heteroatoms selected from N and O. In certain embodiments, ring A is selected from a phenyl or a 5 to 6-membered heterocyclic ring, wherein said heterocycle contains from 1 to 2 ring heteroatoms selected from O and N. In further embodiments, ring A is selected from a phenyl, a pyridine, a thiophene, a furan, a pyrimidine, a pyrazine, a piridazine, a piperidine, a piperazine, a morpholine or a pyrrolidine. In still further embodiments, ring A is selected from a phenyl, a morpholine or a pyrrolidine. In yet further embodiments, ring A is selected from a phenyl, an N-linked morpholine and an N-linked pyrrolidine.

In some embodiments, ring B is selected from a phenyl, a thiophene or a 6-membered heteroaryl. In other embodiments, ring B is selected from a phenyl, a thiophene or a pyridine. In certain embodiments, ring B is a phenyl.

In some embodiments, ring D is selected from a pyrrole, a pyrazole or an imidazole.

In other embodiments, ring D is an imidazole, and x¹ and x³ are N. In certain embodiments, ring D is a pyrazole, and x¹ and x² are N. In further embodiments, ring D is a pyrrole, and x¹ or x³ is N, but both x¹ and x³ are not simultaneously N. In still further embodiments, ring D is a pyrrole, and x¹ is N and x³ is C.

In some embodiments, R² is selected from a halogen, —H, a cyclopropyl ring, a C₁₋₄ alkyl or a C₁₋₄ haloalkyl. In certain embodiments, R² is selected from a C₁₋₄ alkyl or —H. In further embodiments, R² is a methyl.

In some embodiments, R⁴ is selected from a halogen, —NO₂, —R⁶, —OR⁶, —C(O)R⁶, —C(O)OR⁶, —N(R⁶)₂, —S(O)ₚR⁶, —S(O)₂N(R⁶)₂, —NR⁶S(O)₂R⁶, —C(O)N(R⁶)₂ or —NR⁶C(O)R⁶. In other embodiments, R⁴ is a —H, a halogen, —CN, a C₁₋₆ aliphatic radical, a C₃₋₆ cycloaliphatic ring radical, a C₁₋₆ haloaliphatic radical, a phenyl which is optionally substituted by R^{8′} or a benzyl which is optionally substituted by R^{8′}. In certain embodiments, R⁴ is selected from —H, a halogen, —CN, a C₁₋₄ alkyl, a C₁₋₄ haloalkyl, a C₃₋₆ cycloalkyl, a —O(C₁₋₄ alkyl), a —O(C₁₋₄ haloalkyl), a —O(C₃₋₆ cycloalkyl), a —O(phenyl), a —O(substituted phenyl), a —O(benzyl), a —O(substituted benzyl), a —C(O)(C₁₋₄ alkyl), a —C(O)(C₁₋₄ haloalkyl), a —C(O)(C₃₋₆ cycloalkyl), a —C(O)(phenyl), a —C(O)(substituted phenyl), a —C(O)(benzyl), —C(O)(substituted benzyl) or —C(O)H; wherein each of said substituted phenyl or benzyl rings, is substituted by from 0 to 4 instances of R^{8′}. In further embodiments, R⁴ is selected from —H, a halogen, —CN, an ethyl, a methyl, a propyl, a trifluoroethyl, a trifluoromethyl, a cyclopropyl, a cyclopentyl, a cyclohexyl, a cyclopropyloxy, a cyclopentyloxy, a cyclohexyloxy, an ethoxy, a methoxy, a propyloxy, a trifluoromethoxy, a trifluoroethoxy, a benzoyl, a phenyl, a phenyloxy, a methylcarbonyl, an ethylcarbonyl, a trifluoromethylcarbonyl, a trifluoroethylcarbonyl or —C(O)H; wherein each of said benzoyl, phenyl or phenyloxy is independently substituted by from 0 to 4 instances of R^{8′}. In still further embodiments, R⁴ is selected from a —H, a halogen, —CN, an ethyl, a methyl, a propyl, a trifluoroethyl, a trifluoromethyl, a cyclopropyl, a cyclopentyl, a cyclohexyl, phenyl, a benzoyl, a methylcarbonyl, an ethylcarbonyl, a trifluoromethylcarbonyl, a trifluoroethylcarbonyl or a —C(O)H; wherein each of said phenyl and benzoyl groups is independently substituted by from 0 to 4 instances of R^{8′}. In yet further embodiments, R⁴ is selected from —H, iodo, —CN, methyl, 2,2,2-trifluoroethyl, benzoyl, methylcarbonyl, trifluoromethylcarbonyl, —C(O)H or phenyl; wherein said phenyl is independently substituted with from 0 to 2 instances of halogen. In yet further embodiments, R⁴ is a phenyl substituted with from 0 to 2 instances of halogen. In yet further embodiments, R⁴ is a phenyl substituted with from 0 to 2 instances of fluoro. In yet further embodiments, R⁴ is selected from a —H, —CN, a methyl, 2,2,2-trifluoroethyl, a benzoyl, a methylcarbonyl, a trifluoromethylcarbonyl, —C(O)H, a phenyl or a fluorophenyl; wherein said fluorophenyl is substituted with from 0 to 2 instances of fluoro.

In some embodiments, R⁵ is selected from a halogen, —CN, a C₁₋₆ aliphatic radical independently substituted with from 0 to 4 instances of R⁷, a C₃₋₆ cycloaliphatic radical, a phenyl independently substituted with from 0 to 4 instances of R⁸ or a 6-membered heteroaryl independently substituted with from 0 to 4 instances of R^{8′}. In certain embodiments, R⁵ is selected from a halogen, —CN, a C₁₋₆ alkyl independently substituted with from 0 to 4 instances of R⁷, a C₃₋₆ cycloaliphatic, a phenyl independently substituted by from 0 to 4 instances of R⁸ or a 6-membered heteroaryl independently substituted by from 0 to 4 instances of R^{8′}. In further embodiments, R⁵ is selected from a halogen, —CN; a C₁₋₆ alkyl substituted with from 0 to 2 instances of a substituent independently selected from halogen or —OH; a 3 to 6-membered cycloalkyl, a phenyl or a 6-membered heteroaryl; wherein each of said phenyl and 6-membered heteroaryl rings is substituted by from 0 to 3 instances of a substituent independently selected from a halogen, a C₁₋₄ alkyl, a C₁₋₄ haloalkyl, a C₁₋₄ alkoxy, a C₁₋₄ haloalkoxy and —CN. In still further embodiments, R⁵ is selected from a halogen, —CN, an ethyl, a methyl, a propyl, a 3-6 membered cycloalkyl, a phenyl, a pyridinyl or a pyrimidinyl; wherein each said methyl, ethyl and propyl is substituted with from 0 to 4 instances of a halogen or —OH; and wherein each said phenyl, pyridinyl and pyrimidinyl is substituted with from 0 to 4 instances of a substituent selected from a halogen, a C₁₋₂ alkyl, a C₁₋₂ haloalkyl, a C₁₋₂ alkoxy or a C₁₋₂haloalkoxy. In yet further embodiments, R⁵ is selected from —CN, an ethyl, a methyl, a propyl, a cyclopropyl, a cyclopentyl, a cyclohexyl, a phenyl or a pyridinyl; wherein each said methyl, propyl and ethyl is independently substituted with from 0 to 2 instances of a halogen or —OH; wherein said phenyl is independently substituted by from 0 to 2 instances of a halogen or —CF₃; and wherein said pyridinyl is independently substituted by from 0 to 1 instances of a halogen, a C₁₋₂ alkoxy, a C₁₋₂ haloalkoxy or —CF₃. In even further embodiments, R⁵ is selected from a —CN, a 2-hydroxyethyl, a methyl, a cyclopropyl, a cyclopentyl, a cyclohexyl, a phenyl or a pyridinyl; wherein said phenyl is independently substituted by from 0 to 2 instances of fluorine or —CF₃; and wherein said pyridinyl is independently substituted by from 0 to 1 instances of fluoro or chloro. In yet further embodiments, R⁵ is selected from —CN, a methyl, a cyclopropyl, a cyclopentyl, a cyclohexyl, a phenyl, pyridinyl, a 3-chloro-4-pyridinyl or a 3-chloro-2-pyridinyl; wherein said phenyl is independently substituted by from 0 to 2 instances of fluorine or by from 0 to 1 instances of —CF₃.

In some embodiments, each of R¹⁶ and R¹⁷ is independently selected from —H or a methyl or, alternatively, R¹⁶ and R¹⁷, taken together with the carbon atom to which they are attached, form a cyclopropyl ring. In certain embodiments, R¹⁶ and R¹⁷ are both —H.

In some embodiments, L is selected from a methylene, —C(O)— or —S—. In certain embodiments, L is selected from a methylene or —S—.

In some embodiments, o is 0. In certain embodiments, o is 1 or 2 and J^{B} is a halogen.

In some embodiments, L′ is selected from —SO₂— or —CH₂SO₂—. In certain embodiments, L′ is —SO₂—.

In some embodiments, R¹³ is selected from a —H or a C₁₋₆ alkyl. In certain embodiments R¹³ is —H.

Alternatively, in some embodiments the compound having Formula I is not a compound selected from 5-[[6-methoxy-3-(4-methoxybenzoyl)-2-methyl-1H-pyrrolo[2,3-b]pyridin-1-yl]methyl]-α,α-dimethyl-2H-Tetrazole-2-acetic acid [CAS Registry No. 1097838-63-5], 5-[[5-(benzoylamino)-2-thiazolyl]thio]-2H-tetrazole-2-acetic acid [CAS Registry No. 1099441-56-1], 2-butyl-1-[[4-[(2-carboxybenzoyl)amino]phenyl]methyl]-5-chloro-1H-imidazole-4-acetic acid [CAS Registry No. 114798-40-2], and 2-butyl-1-[[4-[(2-carboxybenzoyl)amino]phenyl]methyl]-5-chloro-1H-imidazole-4-acetic acid [CAS Registry No. 114773-45-4], or a pharmaceutically acceptable salt thereof.

Alternatively, the compound having Structural Formula I is not a compound selected from 5-[[6-methoxy-3-(4-methoxybenzoyl)-2-methyl-1H-pyrrolo[2,3-b]pyridin-1-yl]methyl]-α,α-dimethyl-2H-tetrazole-2-acetic acid [CAS Registry No. 1097838-63-5], a derivative of 5-[[6-methoxy-3-(4-methoxybenzoyl)-2-methyl-1H-pyrrolo[2,3-b]pyridin-1-yl]methyl]-α,α-dimethyl-2H-tetrazole-2-acetic acid in which a H atom is replaced with a methyl or ethyl group or a methyl group is replaced with a H atom, 5-[[5-(benzoylamino)-2-thiazolyl]thio]-2H-tetrazole-2-acetic acid [CAS Registry No. 1099441-56-1], a derivative of 5-[[5-(benzoylamino)-2-thiazolyl]thio]-2H-tetrazole-2-acetic acid in which a H atom is replaced with a methyl or ethyl group or a methyl group is replaced with a H atom, 2-butyl-1-[[4-[(2-carboxybenzoyl)amino]phenyl]methyl]-5-chloro-1H-imidazole-4-acetic acid [CAS Registry No. 114798-40-2], a derivative of 2-butyl-1-[[4-[(2-carboxybenzoyl)amino]phenyl]methyl]-5-chloro-1H-imidazole-4-acetic acid in which a H atom is replaced with a methyl or ethyl group or a methyl group is replaced with a H atom, 2-butyl-1-[[4-[(2-carboxybenzoyl)amino]phenyl]methyl]-5-chloro-1H-imidazole-4-acetic acid [CAS Registry No. 114773-45-4], and a derivative of 2-butyl-1-[[4-[(2-carboxybenzoyl)amino]phenyl]methyl]-5-chloro-1H-imidazole-4-acetic acid in which a H atom is replaced with a methyl or ethyl group or a methyl group is replaced with a H atom, or pharmaceutically acceptable salts thereof.

In a second aspect, the compound described above with the further proviso that when ring D is a tetrazole and ring B is a thiazole, L is not —S—.

In a third aspect, the compound described above, with the further proviso that when ring D is an imidazole such that x¹ is C, and x is C—R²; ring B is a phenyl; and L is a methylene; then R² is not —H, a halogen, a C₁₋₆ aliphatic radical or a cyclopropyl ring.

In a fourth aspect, the compound described above, with the further proviso that when D is a tetrazole and L is a methylene, two J^{B} groups are not attached to two vicinal ring B atoms to form a 6-membered heterocycle or a 6-membered heteroaryl ring fused to ring D.

In a fifth aspect, the invention is directed to preventing or lessening the severity of or treating a patient suffering from alopecia or acne comprising administering a compound having any one of structural formulae:

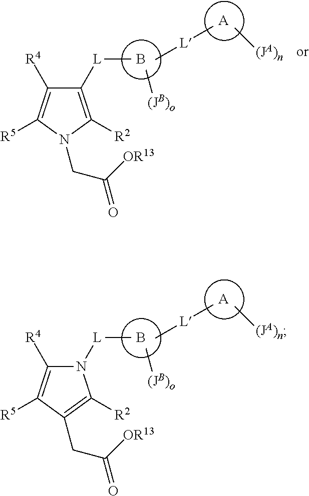

wherein each of the variables can be selected from those described in the embodiments above.

In another aspect, the invention is directed to preventing or lessening the severity of or treating a patient suffering from alopecia or acne comprising administering a compound having any one of structural formulae:

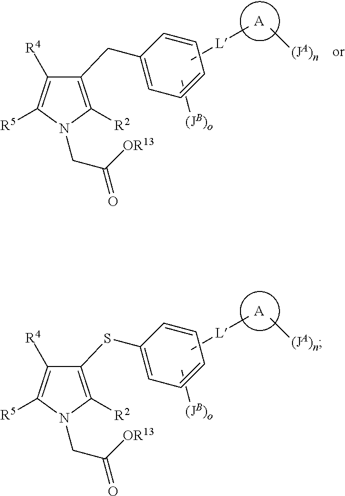

wherein each of the variables can be selected from those described in the embodiments above.

In another aspect, the invention is directed to preventing or lessening the severity of or treating a patient suffering from alopecia or acne comprising administering a compound having any one of structural formulae:

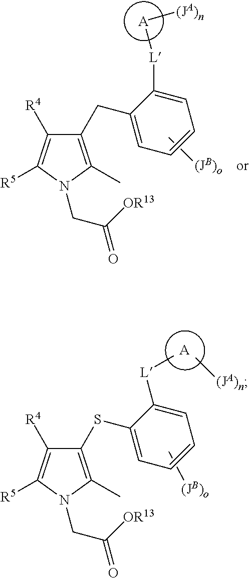

wherein each of the variables can be selected from those described in the embodiments above.

In another aspect, the invention is directed to preventing or lessening the severity of or treating a patient suffering from alopecia or acne comprising administering a compound having any one of structural formulae:

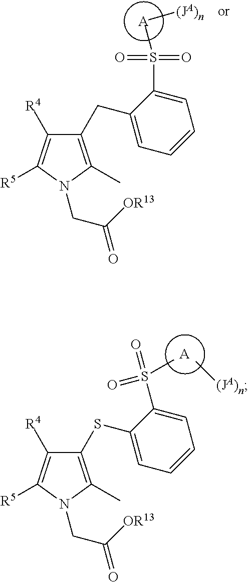

wherein each of the variables can be selected from those described in the embodiments above.

In another aspect, the invention is directed to preventing or lessening the severity of or treating a patient suffering from alopecia or acne comprising administering a compound having any one of structural formulae:

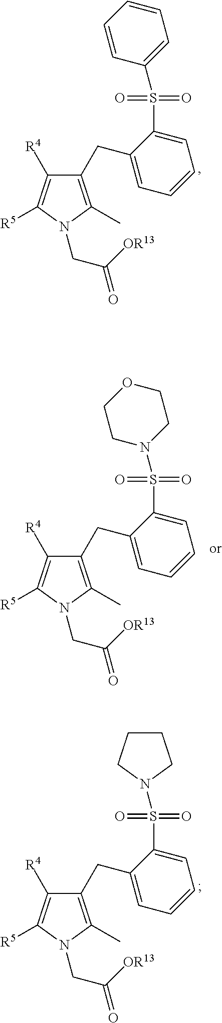

wherein each of the variables can be selected from those described in the embodiments above.

In another aspect, the invention is directed to preventing or lessening the severity of or treating a patient suffering from alopecia or acne comprising administering a compound having any one of structural formulae:

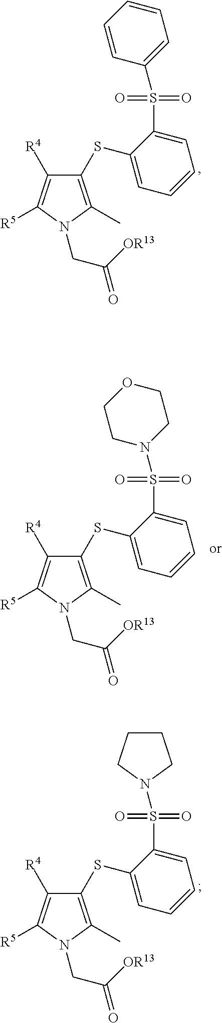

wherein each of the variables can be selected from those described in the embodiments above.

In another aspect, the invention is directed to preventing or lessening the severity of or treating a patient suffering from alopecia or acne comprising administering a compound selected from those depicted in Table I:

TABLE I

| | |
|---|---|
| 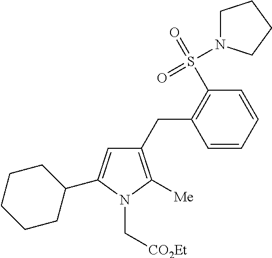 [SMILES: CCOC(=O)Cn1c(C2CCCCC2)cc(Cc2ccccc2S(=O)(=O)N2CCCC2)c1C] | I-49 |
| 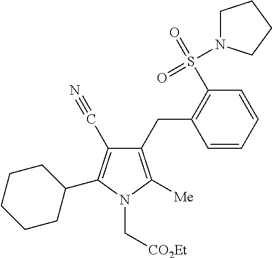 [SMILES: CCOC(=O)Cn1c(C)c(Cc2ccccc2S(=O)(=O)N2CCCC2)c(C#N)c1C1CCCCC1] | I-58 |
| 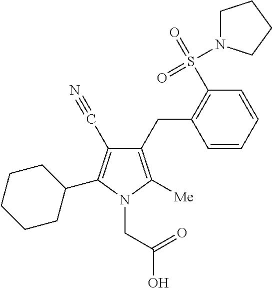 [SMILES: Cc1c(Cc2ccccc2S(=O)(=O)N2CCCC2)c(C#N)c(C2CCCCC2)n1CC(=O)O] | I-14 |
| 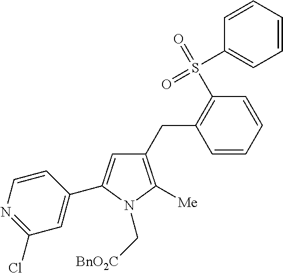 | I-60 |
| 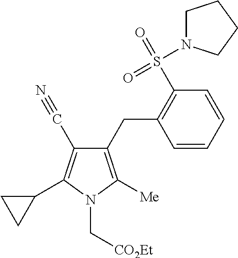 [SMILES: CCOC(=O)Cn1c(C)c(Cc2ccccc2S(=O)(=O)N2CCCC2)c(C#N)c1C1CC1] | I-59 |
| 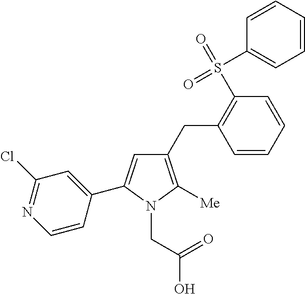 [SMILES: Cc1c(Cc2ccccc2S(=O)(=O)c2ccccc2)cc(-c2ccnc(Cl)c2)n1CC(=O)O] | I-41 |
| 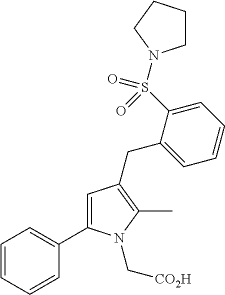 [SMILES: Cc1c(Cc2ccccc2S(=O)(=O)N2CCCC2)cc(-c2ccccc2)n1CC(=O)O] | I-16 |
| 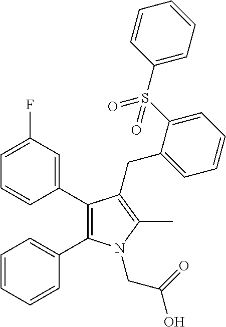 [SMILES: Cc1c(Cc2ccccc2S(=O)(=O)c2ccccc2)c(-c2cccc(F)c2)c(-c2ccccc2)n1CC(=O)O] | I-44 |
| 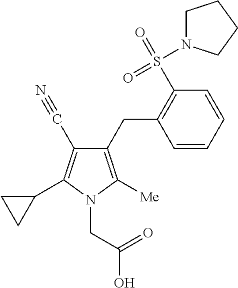 [SMILES: Cc1c(Cc2ccccc2S(=O)(=O)N2CCCC2)c(C#N)c(C2CC2)n1CC(=O)O] | I-33 |
| 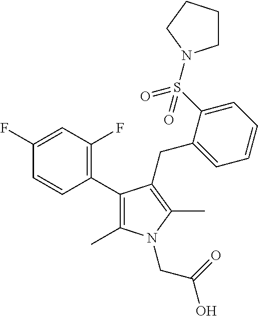 [SMILES: Cc1c(Cc2ccccc2S(=O)(=O)N2CCCC2)c(-c2ccc(F)cc2F)c(C)n1CC(=O)O] | I-36 |
| 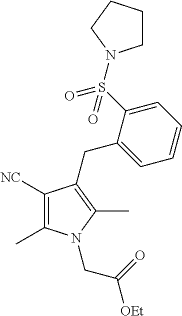 [SMILES: CCOC(=O)Cn1c(C)c(C#N)c(Cc2ccccc2S(=O)(=O)N2CCCC2)c1C] | I-63 |
| 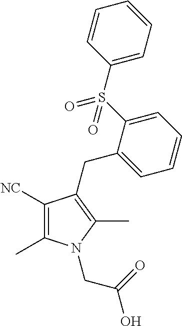 [SMILES: Cc1c(C#N)c(Cc2ccccc2S(=O)(=O)c2ccccc2)c(C)n1CC(=O)O] | I-23 |
| 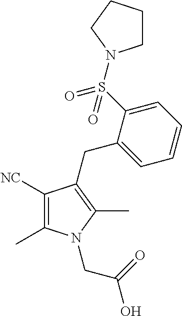 [SMILES: Cc1c(C#N)c(Cc2ccccc2S(=O)(=O)N2CCCC2)c(C)n1CC(=O)O] | I-32 |
| 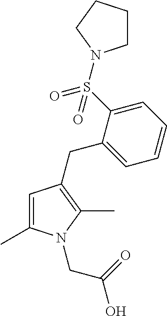 [SMILES: Cc1cc(Cc2ccccc2S(=O)(=O)N2CCCC2)c(C)n1CC(=O)O] | I-9 |
| 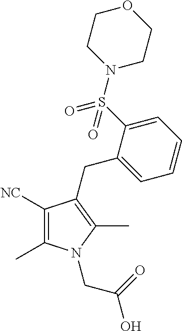 [SMILES: Cc1c(C#N)c(Cc2ccccc2S(=O)(=O)N2CCOCC2)c(C)n1CC(=O)O] | I-46 |
| 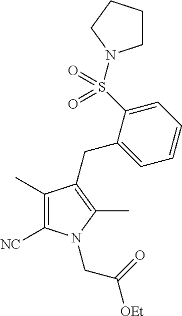 [SMILES: CCOC(=O)Cn1c(C)c(Cc2ccccc2S(=O)(=O)N2CCCC2)c(C)c1C#N] | I-61 |
| 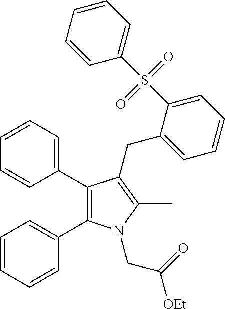 [SMILES: CCOC(=O)Cn1c(C)c(Cc2ccccc2S(=O)(=O)c2ccccc2)c(-c2ccccc2)c1-c1ccccc1] | I-64 |
| 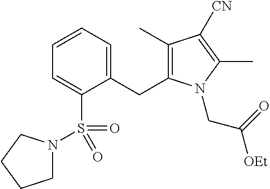 [SMILES: CCOC(=O)Cn1c(C)c(C#N)c(C)c1Cc1ccccc1S(=O)(=O)N1CCCC1] | I-62 |
| 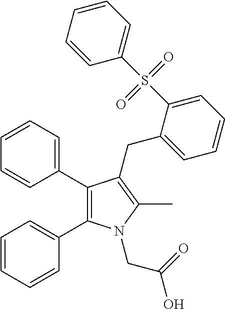 [SMILES: Cc1c(Cc2ccccc2S(=O)(=O)c2ccccc2)c(-c2ccccc2)c(-c2ccccc2)n1CC(=O)O] | I-12 |
| 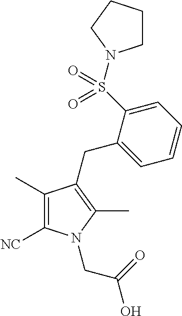 [SMILES: Cc1c(Cc2ccccc2S(=O)(=O)N2CCCC2)c(C)n(CC(=O)O)c1C#N] | I-7 |
| 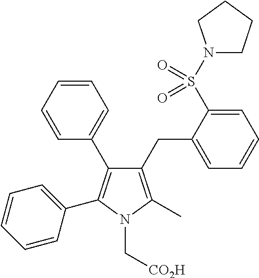 [SMILES: Cc1c(Cc2ccccc2S(=O)(=O)N2CCCC2)c(-c2ccccc2)c(-c2ccccc2)n1CC(=O)O] | I-39 |
| 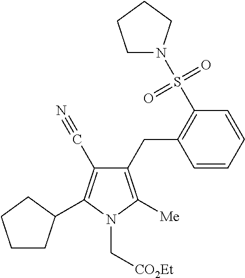 [SMILES: CCOC(=O)Cn1c(C)c(Cc2ccccc2S(=O)(=O)N2CCCC2)c(C#N)c1C1CCCC1] | I-26 |
| 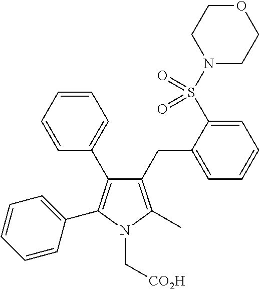 [SMILES: Cc1c(Cc2ccccc2S(=O)(=O)N2CCOCC2)c(-c2ccccc2)c(-c2ccccc2)n1CC(=O)O] | I-40 |
| 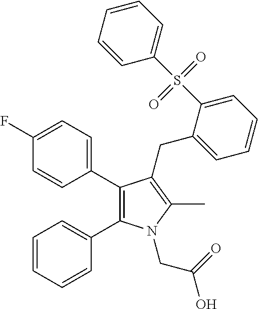 [SMILES: Cc1c(Cc2ccccc2S(=O)(=O)c2ccccc2)c(-c2ccc(F)cc2)c(-c2ccccc2)n1CC(=O)O] | I-37 |
| 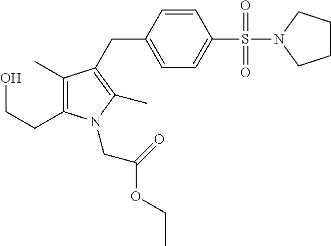 [SMILES: CCOC(=O)Cn1c(C)c(Cc2ccc(S(=O)(=O)N3CCCC3)cc2)c(C)c1CCO] | I-65 |
| 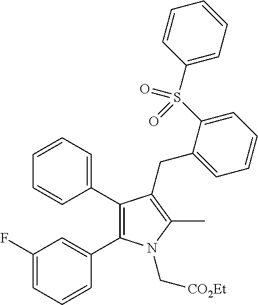 [SMILES: CCOC(=O)Cn1c(C)c(Cc2ccccc2S(=O)(=O)c2ccccc2)c(-c2ccccc2)c1-c1cccc(F)c1] | I-67 |
| 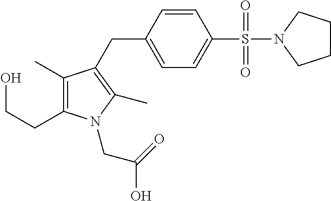 [SMILES: Cc1c(Cc2ccc(S(=O)(=O)N3CCCC3)cc2)c(C)n(CC(=O)O)c1CCO] | I-20 |
| 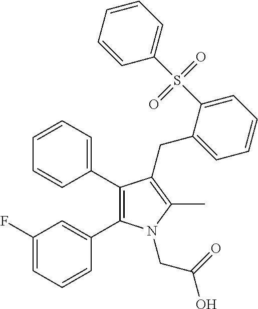 [SMILES: Cc1c(Cc2ccccc2S(=O)(=O)c2ccccc2)c(-c2ccccc2)c(-c2cccc(F)c2)n1CC(=O)O] | I-38 |
| 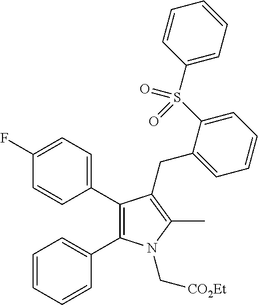 [SMILES: CCOC(=O)Cn1c(C)c(Cc2ccccc2S(=O)(=O)c2ccccc2)c(-c2ccc(F)cc2)c1-c1ccccc1] | I-66 |
| 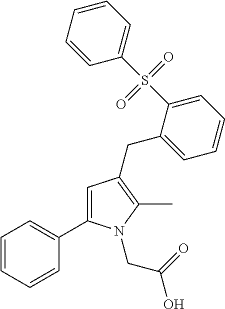 [SMILES: Cc1c(Cc2ccccc2S(=O)(=O)c2ccccc2)cc(-c2ccccc2)n1CC(=O)O] | I-6 |
| 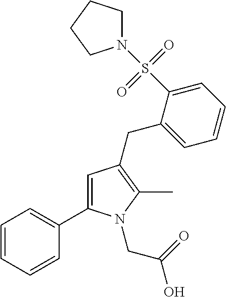 [SMILES: Cc1c(Cc2ccccc2S(=O)(=O)N2CCCC2)cc(-c2ccccc2)n1CC(=O)O] | I-5 |
| 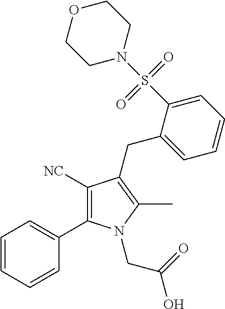 [SMILES: Cc1c(Cc2ccccc2S(=O)(=O)N2CCOCC2)c(C#N)c(-c2ccccc2)n1CC(=O)O] | I-3 |
| 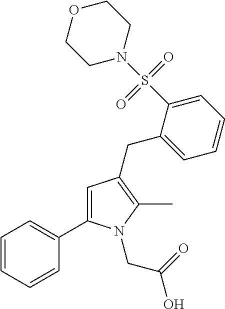 [SMILES: Cc1c(Cc2ccccc2S(=O)(=O)N2CCOCC2)cc(-c2ccccc2)n1CC(=O)O] | I-13 |
| 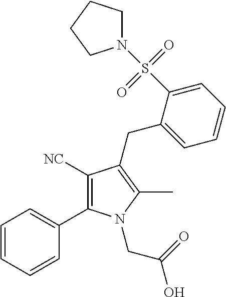 [SMILES: Cc1c(Cc2ccccc2S(=O)(=O)N2CCCC2)c(C#N)c(-c2ccccc2)n1CC(=O)O] | I-11 |
| 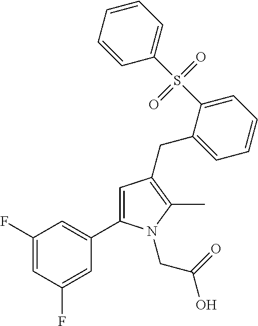 [SMILES: Cc1c(Cc2ccccc2S(=O)(=O)c2ccccc2)cc(-c2cc(F)cc(F)c2)n1CC(=O)O] | I-43 |
| 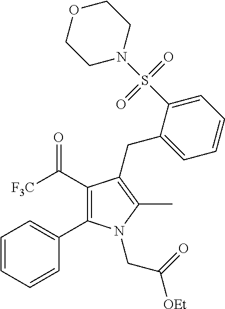 [SMILES: CCOC(=O)Cn1c(C)c(Cc2ccccc2S(=O)(=O)N2CCOCC2)c(C(=O)C(F)(F)F)c1-c1ccccc1] | I-21 |
| 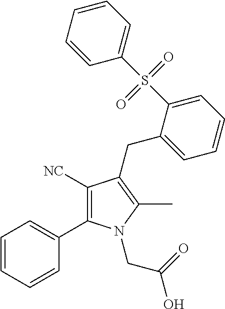 [SMILES: Cc1c(Cc2ccccc2S(=O)(=O)c2ccccc2)c(C#N)c(-c2ccccc2)n1CC(=O)O] | I-4 |
| 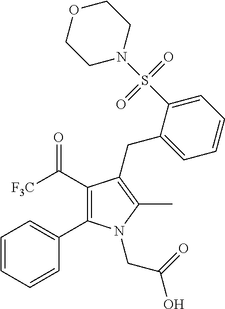 [SMILES: Cc1c(Cc2ccccc2S(=O)(=O)N2CCOCC2)c(C(=O)C(F)(F)F)c(-c2ccccc2)n1CC(=O)O] | I-50 |
| 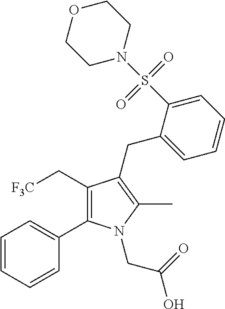 [SMILES: Cc1c(Cc2ccccc2S(=O)(=O)N2CCOCC2)c(CC(F)(F)F)c(-c2ccccc2)n1CC(=O)O] | I-45 |
| 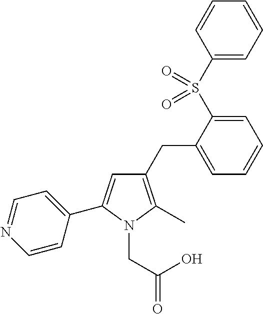 [SMILES: Cc1c(Cc2ccccc2S(=O)(=O)c2ccccc2)cc(-c2ccncc2)n1CC(=O)O] | I-1 |
| 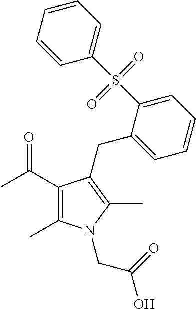 [SMILES: CC(=O)c1c(Cc2ccccc2S(=O)(=O)c2ccccc2)c(C)n(CC(=O)O)c1C] | I-52 |
| 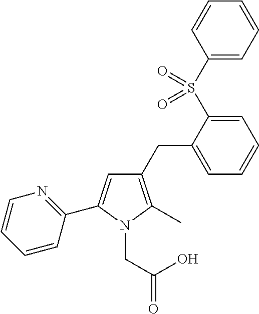 [SMILES: Cc1c(Cc2ccccc2S(=O)(=O)c2ccccc2)cc(-c2ccccn2)n1CC(=O)O] | I-17 |
| 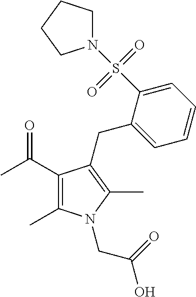 [SMILES: CC(=O)c1c(Cc2ccccc2S(=O)(=O)N2CCCC2)c(C)n(CC(=O)O)c1C] | I-53 |
| 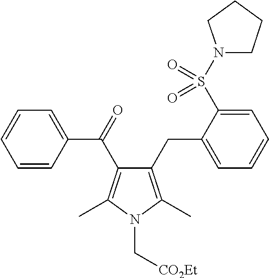 [SMILES: CCOC(=O)Cn1c(C)c(Cc2ccccc2S(=O)(=O)N2CCCC2)c(C(=O)c2ccccc2)c1C] | I-68 |
| 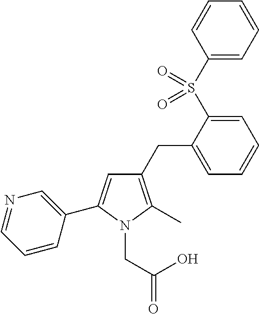 [SMILES: Cc1c(Cc2ccccc2S(=O)(=O)c2ccccc2)cc(-c2cccnc2)n1CC(=O)O] | I-2 |
| 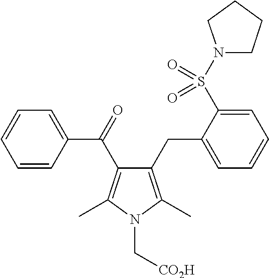 [SMILES: Cc1c(Cc2ccccc2S(=O)(=O)N2CCCC2)c(C(=O)c2ccccc2)c(C)n1CC(=O)O] | I-55 |
| 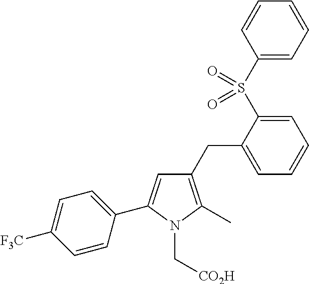 [SMILES: Cc1c(Cc2ccccc2S(=O)(=O)c2ccccc2)cc(-c2ccc(C(F)(F)F)cc2)n1CC(=O)O] | I-25 |
| 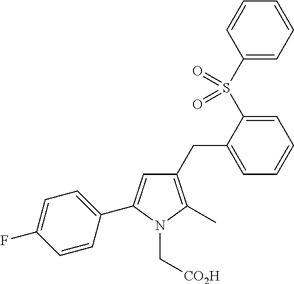 [SMILES: Cc1c(Cc2ccccc2S(=O)(=O)c2ccccc2)cc(-c2ccc(F)cc2)n1CC(=O)O] | I-15 |
| 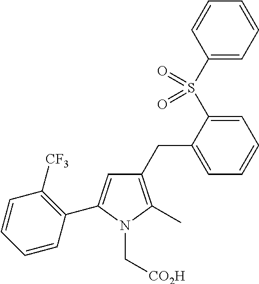 [SMILES: Cc1c(Cc2ccccc2S(=O)(=O)c2ccccc2)cc(-c2ccccc2C(F)(F)F)n1CC(=O)O] | I-27 |
| 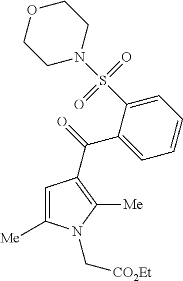 [SMILES: CCOC(=O)Cn1c(C)cc(C(=O)c2ccccc2S(=O)(=O)N2CCOCC2)c1C] | I-69 |
| 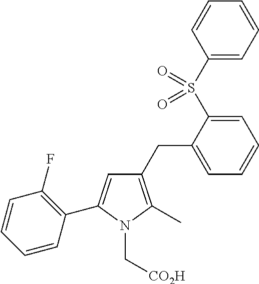 [SMILES: Cc1c(Cc2ccccc2S(=O)(=O)c2ccccc2)cc(-c2ccccc2F)n1CC(=O)O] | I-18 |
| 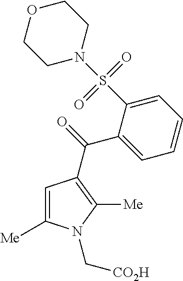 [SMILES: Cc1cc(C(=O)c2ccccc2S(=O)(=O)N2CCOCC2)c(C)n1CC(=O)O] | I-56 |
| 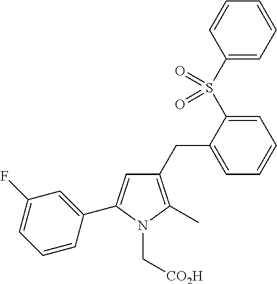 [SMILES: Cc1c(Cc2ccccc2S(=O)(=O)c2ccccc2)cc(-c2cccc(F)c2)n1CC(=O)O] | I-8 |
| 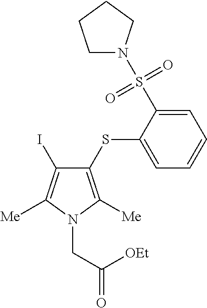 [SMILES: CCOC(=O)Cn1c(C)c(I)c(Sc2ccccc2S(=O)(=O)N2CCCC2)c1C] | I-70 |
| 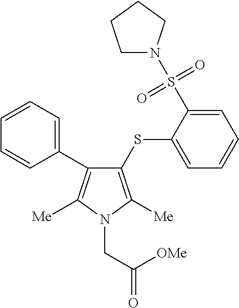 [SMILES: COC(=O)Cn1c(C)c(Sc2ccccc2S(=O)(=O)N2CCCC2)c(-c2ccccc2)c1C] | I-71 |
| 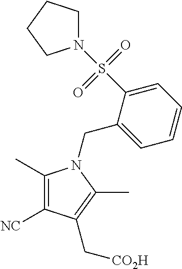 [SMILES: Cc1c(C#N)c(CC(=O)O)c(C)n1Cc1ccccc1S(=O)(=O)N1CCCC1] | I-73 |
| 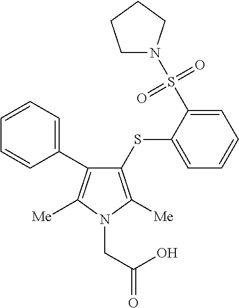 [SMILES: Cc1c(Sc2ccccc2S(=O)(=O)N2CCCC2)c(-c2ccccc2)c(C)n1CC(=O)O] | I-30 |
| 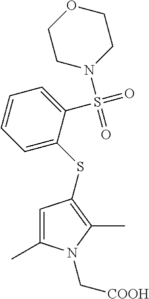 [SMILES: Cc1cc(Sc2ccccc2S(=O)(=O)N2CCOCC2)c(C)n1CC(=O)O] | I-29 |
| 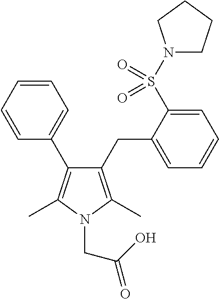 [SMILES: Cc1c(Cc2ccccc2S(=O)(=O)N2CCCC2)c(-c2ccccc2)c(C)n1CC(=O)O] | I-57 |
| 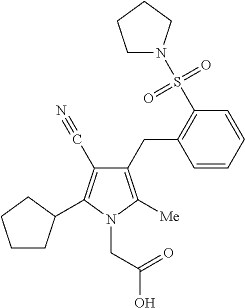 [SMILES: Cc1c(Cc2ccccc2S(=O)(=O)N2CCCC2)c(C#N)c(C2CCCC2)n1CC(=O)O] | I-34 |
| 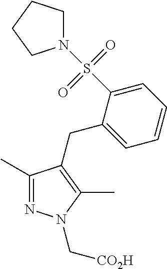 [SMILES: Cc1nn(CC(=O)O)c(C)c1Cc1ccccc1S(=O)(=O)N1CCCC1] | I-72 |
| 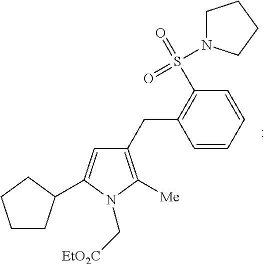 [SMILES: CCOC(=O)Cn1c(C2CCCC2)cc(Cc2ccccc2S(=O)(=O)N2CCCC2)c1C] | I-74 |

In one aspect, the present invention provides a method for preventing or lessening the severity of or treating a patient suffering from alopecia or acne comprising administering to said patient a therapeutically effective amount of a compound of Formula II, or a pharmaceutically acceptable salt thereof; or a pharmaceutical composition comprising the compound of Formula II, or a pharmaceutically acceptable salt thereof, and a pharmaceutically acceptable carrier; wherein the compound of Formula II is represented by the following structural formula

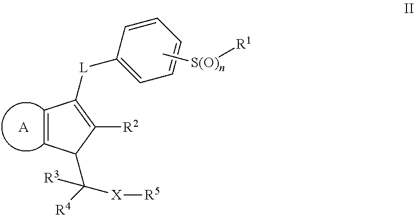

Alternatively, A may be a fused cycloheptyl ring optionally substituted with up to eight instances of R⁸, independently selected. The compounds of this embodiment may be represented by the formula:

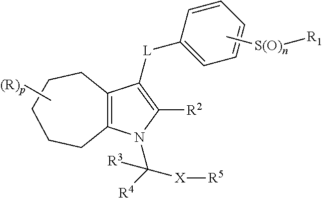

in which p is zero or an integer from 1 to 8.

In another embodiment, A may be a fused cyclopentyl ring optionally substituted with up to six instances of R⁸, independently selected. The compounds of this embodiment may be represented by the formula:

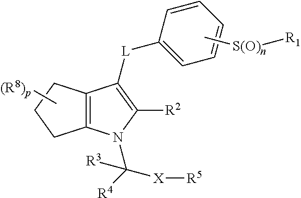

in which q is zero or an integer from 1 to 6.

In another embodiment, A may be a fused cyclohexyl ring optionally substituted with up to eight instances of R⁸, independently selected. The compounds of this embodiment may be represented by the formula:

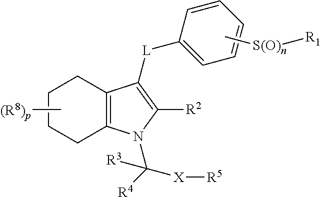

In certain embodiments, two R⁸ may each be a methyl residue attached to the same ring carbon, such as in compounds of formula

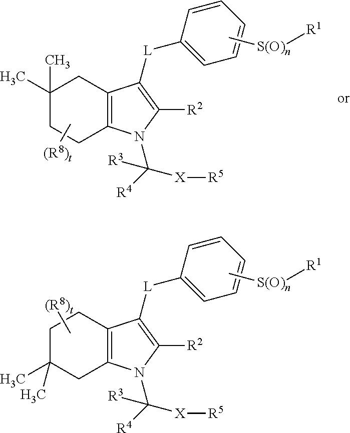

in which t is zero or an integer from 1 to 4. In a particular embodiment, t is zero. In another embodiment, t is 1 and R⁸ is oxo.

In certain embodiments X may be chosen from a direct bond, —CH₂— and —CH₂CH₂—. Alternatively, X may be a direct bond.

In certain embodiments R² may be chosen from hydrogen, fluoro, methyl, ethyl and trifluoromethyl. Alternatively, R² may be methyl.

In certain embodiments, R³ and R⁴ may be taken together to form a cyclopropyl ring. In other embodiments R³ and R⁴ may each be independently selected from hydrogen and methyl and the methyl may be substituted with 1-3 instances of halogen, particularly fluoro. In other embodiments, R³ and R⁴ may each be hydrogen.

Preferably, L may be chosen from —CH₂—, —O—, —S—, —SO— and —SO₂—. More preferably, L is —CH₂—.

Preferably, X is a direct bond and R⁵ is selected from the group consisting of C(O)OR⁷, C(O)N(R⁷)₂, C(O)NHOR⁷ or C(O)NHSR⁷, and R⁷ may be H or (C₁₋₄)alkyl. More preferably, R⁵ may be either C(O)OH or C(O)O(C₁₋₄)alkyl. When R⁵ is C(O)OH, the compounds of Formula II may be presented in the form of salts. Salts containing pharmaceutically acceptable cations are preferred for compositions and formulations that will be administered to humans.

In certain embodiments, R¹ is preferably —NR⁶(C₁-C₆)alkyl and R⁶ is preferably hydrogen or methyl. In other embodiments R¹ may be a non-aromatic 3-8 membered heterocycle, phenyl, or a non-aromatic 3-8 membered carbocycle, and the heterocycle, phenyl or carbocycle may be substituted with up to four instances of R⁹. In some embodiments, R¹ may be phenyl or a non-aromatic 3-8 membered carbocycle; in others, R¹ is a non-aromatic 3-8 membered heterocycle; in others, R¹ is a non-aromatic 5-7 membered heterocycle, optionally substituted with one to three instances of R⁹. In particular embodiments, R¹ is an N-attached pyrrolidine, piperidine, piperazine, azepine or morpholine, optionally substituted with one to three instances of R⁹, wherein each R⁹ is independently selected from (C₁-C₄)alkyl and (C₁-C₄)haloalkyl. In certain embodiments, R¹ is an N-attached piperazine of formula

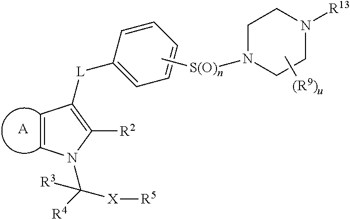

in which R¹³ is chosen from hydrogen, (C₁-C₄)alkyl, (C₁-C₄)alkylcarbonyl and (C₁-C₄)alkoxycarbonyl and u is zero, one or two. In other embodiments, R¹ is an N-attached morpholine of formula

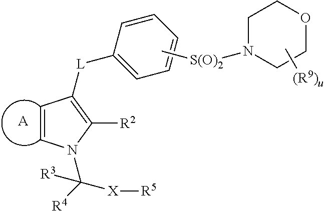

and u is zero, one or two. The compound may have one of the following formulae:

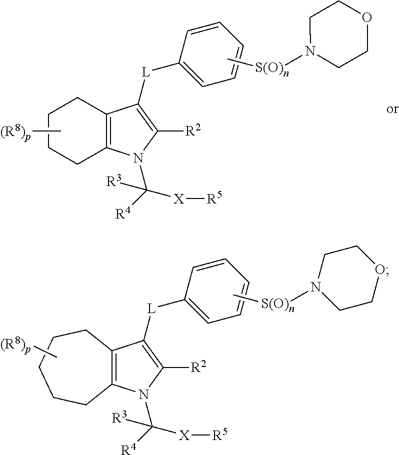

wherein p may be zero or an integer from 1 to 3. The compound may have the formula:

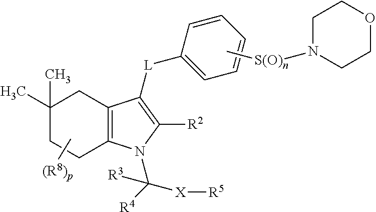

and p may be zero or one.

In many embodiments shown above, —S(O)ₙR¹ is attached para or ortho to L on the phenyl ring. As before, R⁵ may be C(O)OH or C(O)O(C₁₋₄)alkyl, and when R⁵ is C(O)OH the compounds of Formula II may be presented in the form of salts.

Compounds of Formula II may comprise compounds of formula II in which

A is chosen from a cyclopentyl ring, a cycloheptyl ring and a dimethylcyclohexyl ring;
R² may be methyl;
R³ and R⁴ may be hydrogen;

### R⁵ may be COOH;

X may be a direct bond;

### L may be —CH₂—;

n may be 2; and
R¹ may be chosen from
(a) pyrrolidinyl, piperidinyl, azepinyl or morpholinyl;
(b) piperazine-1-yl substituted at the 4 position with (C₁-C₄)alkylcarbonyl or (C₁-C₄)alkoxycarbonyl;
(c) —NR⁶(C₁-C₆)alkyl wherein R⁶ is hydrogen or methyl; and
(d) phenyl and substituted phenyl.

In another embodiment, the invention comprises administration of compounds of formula III

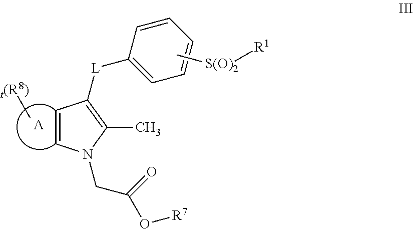

in which
A may be chosen from a cyclopentyl ring, a cycloheptyl ring, a cyclohexyl ring, and a dimethylcyclohexyl ring;
R⁷ may be hydrogen or (C₁₋₄) alkyl;

### L may be —CH₂— or —S(O)ₘ—;

m may be 0 or 2;
R⁸ may be oxo;
t may be 0 or 1; and
R¹ may be chosen from
(a) pyrrolidinyl, piperidinyl, azepinyl or morpholinyl;
(b) piperazine-1-yl substituted at the 4 position with (C₁-C₄)alkylcarbonyl or (C₁-C₄)alkoxycarbonyl;
(c) —NR⁶(C₁-C₆)alkyl wherein R⁶ is hydrogen or methyl; or
(d) phenyl and substituted phenyl.

In a further embodiment of compounds of formula III, A may be a cyclohexyl ring or a dimethylcyclohexyl ring.

In a further embodiment, the invention may comprise preventing or lessening the severity of or treating a patient suffering from alopecia or acne comprising administering compounds of formulae III′ and III″:

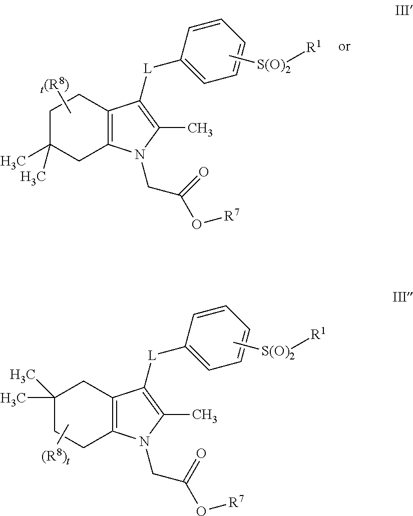

In the compounds of any of formulae III, III′ or III″, L may be —CH₂—.

In the compounds of any of formulae III, III′ or III″, t may be one and R⁸ may be oxo. In yet a further embodiment, the invention comprises compounds of formula IV:

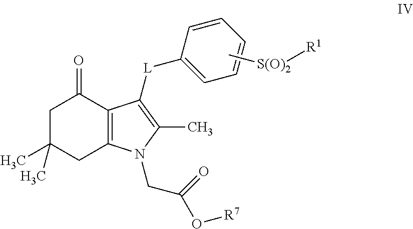

In the compounds of any of formulae III, III′, III″ or IVI, R¹ may be chosen from pyrrolidinyl, piperidinyl, azepinyl, morpholinyl or phenyl. Alternatively, in the compounds of any of formulae III, III′, III″ or IV, R¹ may be chosen from pyrrolidine or morpholine.

In the compounds of any of formulae III, III′, III″ or IVI, —S(O)₂R¹ may be attached para or ortho to L on the phenyl ring. As before, when R⁷ is hydrogen, the compounds of Formula II may be presented in the form of salts.

In one embodiment, of the compounds III the fused cyclohexyl ring may be substituted with up to eight instances of R⁸ independently selected from the group consisting of halogen and (C₁-C₄)alkyl. In this embodiment the compound has one of the following two formulae:

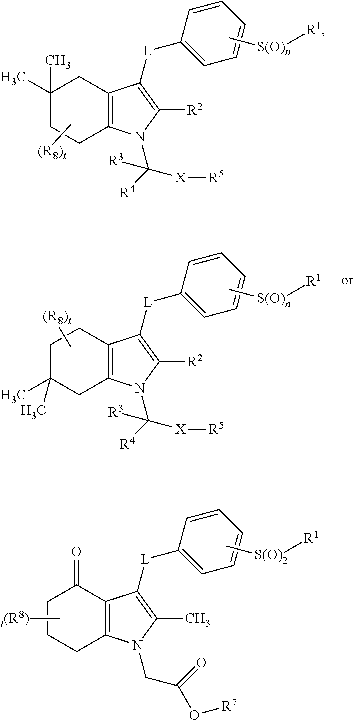

wherein t is 0 or an integer of from 1 to 6. Preferably R⁸ is selected from the group consisting of fluoro and methyl. Preferably, t is an integer from 1 to 4. In this embodiment, preferably the compound has the following structure:

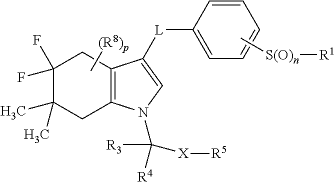

wherein p is 0 or an integer from 1 to 4. Most preferably, p is 0 or R⁸ is an Oxo.

Further examples of embodiments of the invention are shown in Table 2 below.

TABLE 2

| | |
|---|---|
| 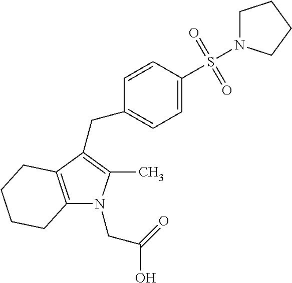 [SMILES: Cc1c(Cc2ccc(S(=O)(=O)N3CCCC3)cc2)c2c(n1CC(=O)O)CCCC2] | I-1 |
| 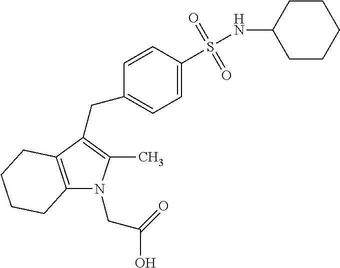 [SMILES: Cc1c(Cc2ccc(S(=O)(=O)NC3CCCCC3)cc2)c2c(n1CC(=O)O)CCCC2] | I-2 |
| 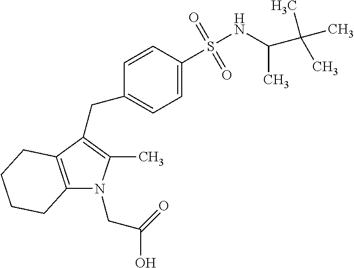 [SMILES: Cc1c(Cc2ccc(S(=O)(=O)NC(C)C(C)(C)C)cc2)c2c(n1CC(=O)O)CCCC2] | I-3 |
| 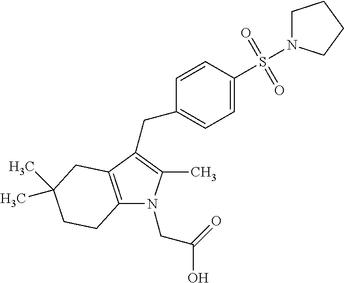 [SMILES: Cc1c(Cc2ccc(S(=O)(=O)N3CCCC3)cc2)c2c(n1CC(=O)O)CCC(C)(C)C2] | I-4 |
| 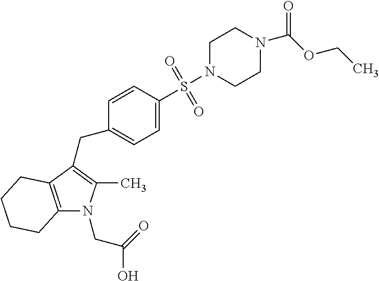 [SMILES: CCOC(=O)N1CCN(S(=O)(=O)c2ccc(Cc3c4c(n(CC(=O)O)c3C)CCCC4)cc2)CC1] | I-5 |
| 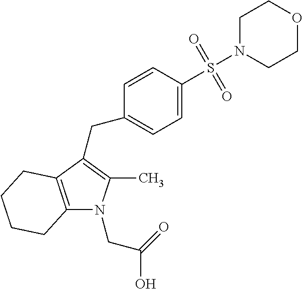 [SMILES: Cc1c(Cc2ccc(S(=O)(=O)N3CCOCC3)cc2)c2c(n1CC(=O)O)CCCC2] | I-6 |
| 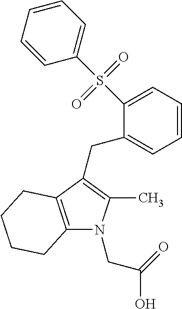 [SMILES: Cc1c(Cc2ccccc2S(=O)(=O)c2ccccc2)c2c(n1CC(=O)O)CCCC2] | I-7 |
| 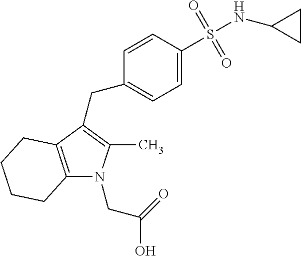 [SMILES: Cc1c(Cc2ccc(S(=O)(=O)NC3CC3)cc2)c2c(n1CC(=O)O)CCCC2] | I-8 |
| 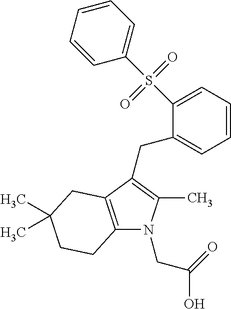 [SMILES: Cc1c(Cc2ccccc2S(=O)(=O)c2ccccc2)c2c(n1CC(=O)O)CCC(C)(C)C2] | I-9 |
| 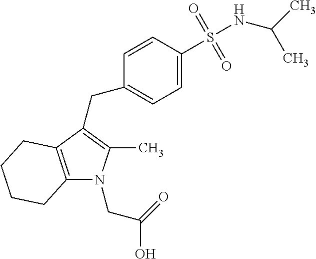 [SMILES: Cc1c(Cc2ccc(S(=O)(=O)NC(C)C)cc2)c2c(n1CC(=O)O)CCCC2] | I-10 |
| 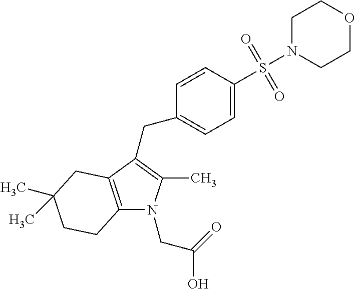 [SMILES: Cc1c(Cc2ccc(S(=O)(=O)N3CCOCC3)cc2)c2c(n1CC(=O)O)CCC(C)(C)C2] | I-11 |
| 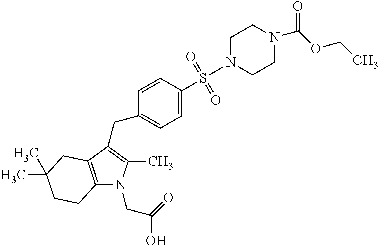 [SMILES: CCOC(=O)N1CCN(S(=O)(=O)c2ccc(Cc3c4c(n(CC(=O)O)c3C)CCC(C)(C)C4)cc2)CC1] | I-12 |
| 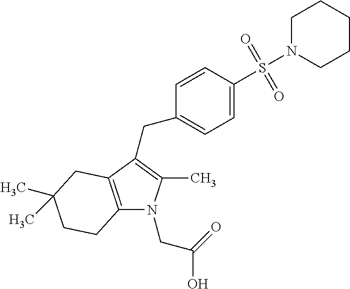 [SMILES: Cc1c(Cc2ccc(S(=O)(=O)N3CCCCC3)cc2)c2c(n1CC(=O)O)CCC(C)(C)C2] | I-13 |
| 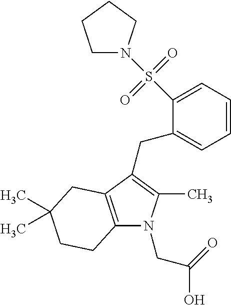 [SMILES: Cc1c(Cc2ccccc2S(=O)(=O)N2CCCC2)c2c(n1CC(=O)O)CCC(C)(C)C2] | I-14 |
| 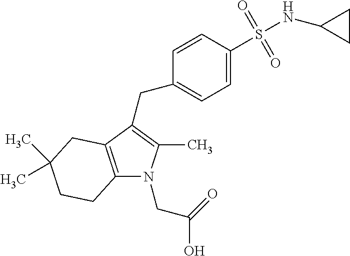 [SMILES: Cc1c(Cc2ccc(S(=O)(=O)NC3CC3)cc2)c2c(n1CC(=O)O)CCC(C)(C)C2] | I-15 |
| 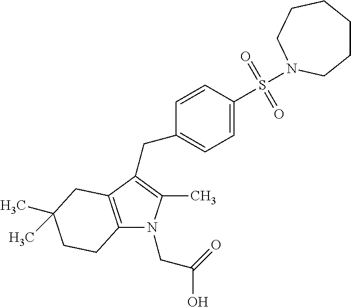 [SMILES: Cc1c(Cc2ccc(S(=O)(=O)N3CCCCCC3)cc2)c2c(n1CC(=O)O)CCC(C)(C)C2] | I-16 |
| 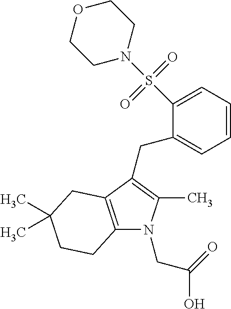 [SMILES: Cc1c(Cc2ccccc2S(=O)(=O)N2CCOCC2)c2c(n1CC(=O)O)CCC(C)(C)C2] | I-17 |
| 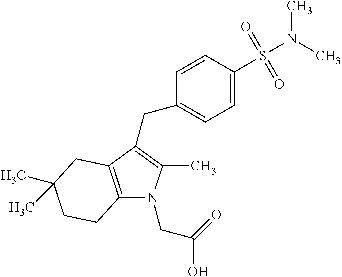 | I-18 |
| 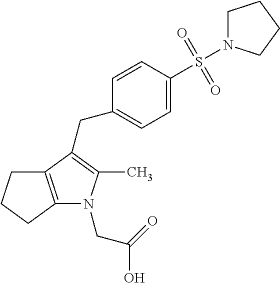 [SMILES: Cc1c(Cc2ccc(S(=O)(=O)N3CCCC3)cc2)c2c(n1CC(=O)O)CCC2] | I-19 |
| 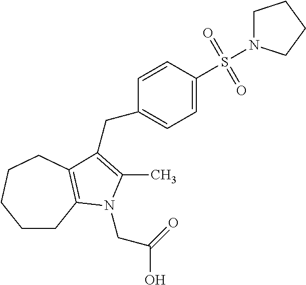 [SMILES: Cc1c(Cc2ccc(S(=O)(=O)N3CCCC3)cc2)c2c(n1CC(=O)O)CCCCC2] | I-20 |
| 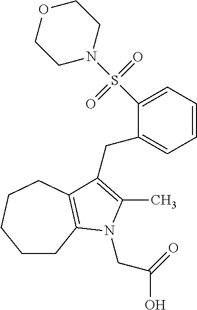 [SMILES: Cc1c(Cc2ccccc2S(=O)(=O)N2CCOCC2)c2c(n1CC(=O)O)CCCCC2] | I-21 |
| 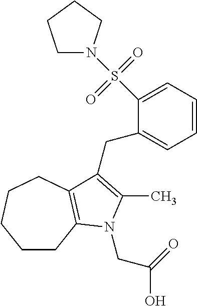 [SMILES: Cc1c(Cc2ccccc2S(=O)(=O)N2CCCC2)c2c(n1CC(=O)O)CCCCC2] | I-22 |
| 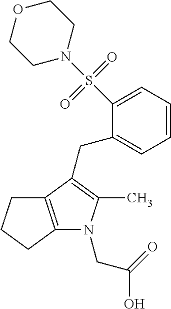 [SMILES: Cc1c(Cc2ccccc2S(=O)(=O)N2CCOCC2)c2c(n1CC(=O)O)CCC2] | I-23 |
| 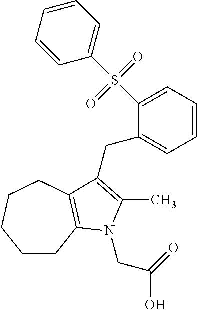 [SMILES: Cc1c(Cc2ccccc2S(=O)(=O)c2ccccc2)c2c(n1CC(=O)O)CCCCC2] | I-24 |
| 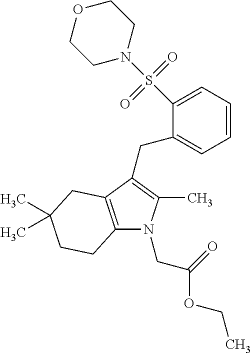 [SMILES: CCOC(=O)Cn1c(C)c(Cc2ccccc2S(=O)(=O)N2CCOCC2)c2c1CCC(C)(C)C2] | I-25 |
| 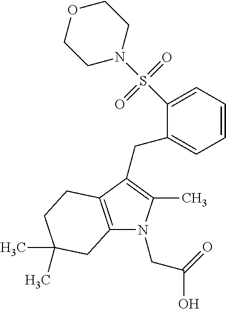 [SMILES: Cc1c(Cc2ccccc2S(=O)(=O)N2CCOCC2)c2c(n1CC(=O)O)CC(C)(C)CC2] | I-26 |
| 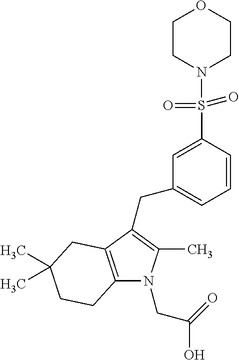 [SMILES: Cc1c(Cc2cccc(S(=O)(=O)N3CCOCC3)c2)c2c(n1CC(=O)O)CCC(C)(C)C2] | I-27 |
| 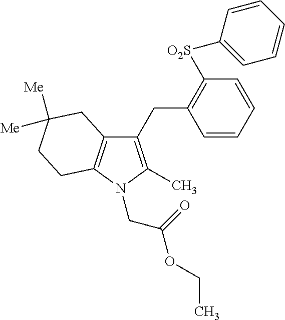 [SMILES: CCOC(=O)Cn1c(C)c(Cc2ccccc2S(=O)(=O)c2ccccc2)c2c1CCC(C)(C)C2] | I-28 |
| 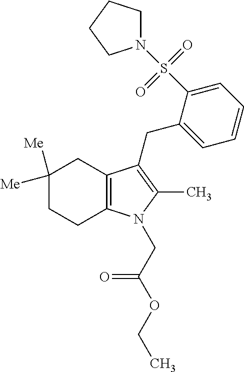 [SMILES: CCOC(=O)Cn1c(C)c(Cc2ccccc2S(=O)(=O)N2CCCC2)c2c1CCC(C)(C)C2] | I-29 |
| 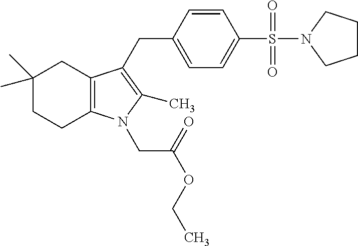 [SMILES: CCOC(=O)Cn1c(C)c(Cc2ccc(S(=O)(=O)N3CCCC3)cc2)c2c1CCC(C)(C)C2] | I-30 |
| 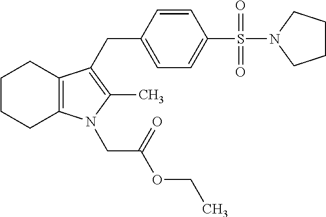 [SMILES: CCOC(=O)Cn1c(C)c(Cc2ccc(S(=O)(=O)N3CCCC3)cc2)c2c1CCCC2] | I-31 |
| 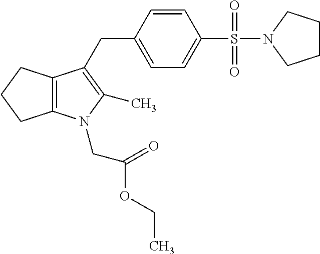 [SMILES: CCOC(=O)Cn1c(C)c(Cc2ccc(S(=O)(=O)N3CCCC3)cc2)c2c1CCC2] | I-32 |
| 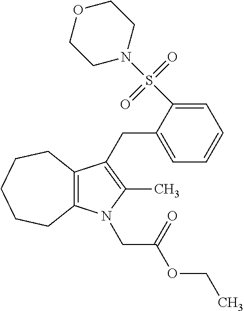 [SMILES: CCOC(=O)Cn1c(C)c(Cc2ccccc2S(=O)(=O)N2CCOCC2)c2c1CCCCC2] | I-33 |
| 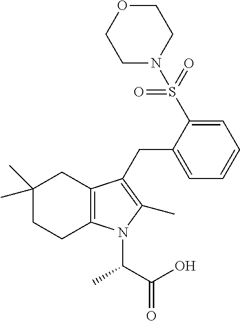 [SMILES: Cc1c(Cc2ccccc2S(=O)(=O)N2CCOCC2)c2c(n1[C@@H](C)C(=O)O)CCC(C)(C)C2] | I-34 |
| 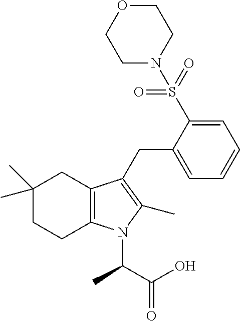 [SMILES: Cc1c(Cc2ccccc2S(=O)(=O)N2CCOCC2)c2c(n1[C@H](C)C(=O)O)CCC(C)(C)C2] | I-35 |
| 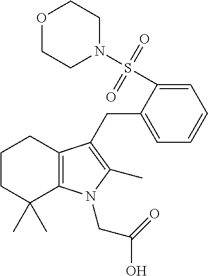 [SMILES: Cc1c(Cc2ccccc2S(=O)(=O)N2CCOCC2)c2c(n1CC(=O)O)C(C)(C)CCC2] | I-36 |
| 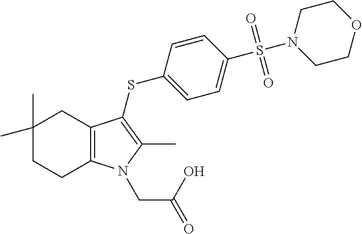 [SMILES: Cc1c(Sc2ccc(S(=O)(=O)N3CCOCC3)cc2)c2c(n1CC(=O)O)CCC(C)(C)C2] | I-37 |
| 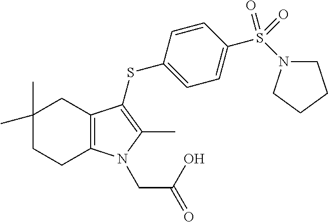 [SMILES: Cc1c(Sc2ccc(S(=O)(=O)N3CCCC3)cc2)c2c(n1CC(=O)O)CCC(C)(C)C2] | I-38 |
| 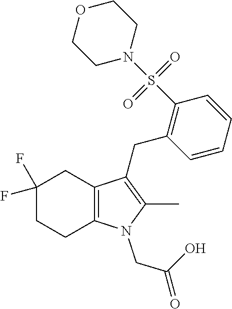 [SMILES: Cc1c(Cc2ccccc2S(=O)(=O)N2CCOCC2)c2c(n1CC(=O)O)CCC(F)(F)C2] | I-39 |
| 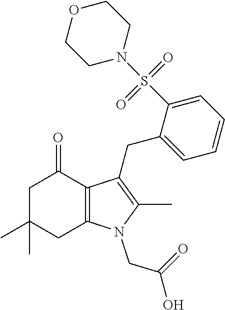 [SMILES: Cc1c(Cc2ccccc2S(=O)(=O)N2CCOCC2)c2c(n1CC(=O)O)CC(C)(C)CC2=O] | I-40 |
| 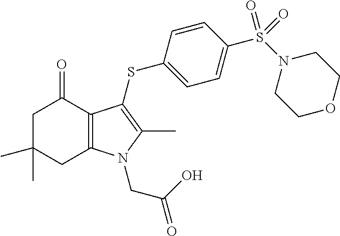 [SMILES: Cc1c(Sc2ccc(S(=O)(=O)N3CCOCC3)cc2)c2c(n1CC(=O)O)CC(C)(C)CC2=O] | I-41 |
| 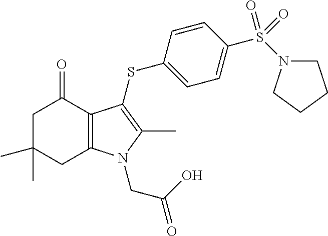 [SMILES: Cc1c(Sc2ccc(S(=O)(=O)N3CCCC3)cc2)c2c(n1CC(=O)O)CC(C)(C)CC2=O] | I-42 |
| 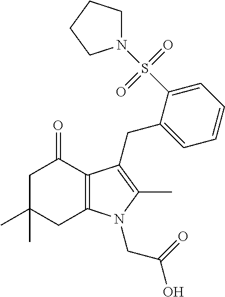 [SMILES: Cc1c(Cc2ccccc2S(=O)(=O)N2CCCC2)c2c(n1CC(=O)O)CC(C)(C)CC2=O] | I-43 |
| 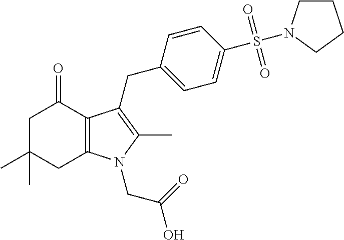 [SMILES: Cc1c(Cc2ccc(S(=O)(=O)N3CCCC3)cc2)c2c(n1CC(=O)O)CC(C)(C)CC2=O] | I-44 |
| 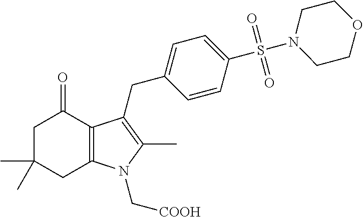 [SMILES: Cc1c(Cc2ccc(S(=O)(=O)N3CCOCC3)cc2)c2c(n1CC(=O)O)CC(C)(C)CC2=O] | I-45 |
| 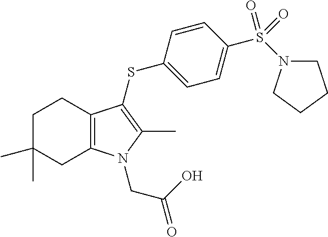 [SMILES: Cc1c(Sc2ccc(S(=O)(=O)N3CCCC3)cc2)c2c(n1CC(=O)O)CC(C)(C)CC2] | I-46 |
| 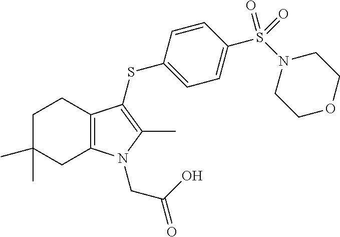 [SMILES: Cc1c(Sc2ccc(S(=O)(=O)N3CCOCC3)cc2)c2c(n1CC(=O)O)CC(C)(C)CC2] | I-47 |
| 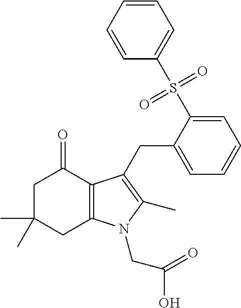 [SMILES: Cc1c(Cc2ccccc2S(=O)(=O)c2ccccc2)c2c(n1CC(=O)O)CC(C)(C)CC2=O] | I-48 |
| 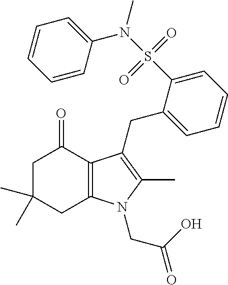 [SMILES: Cc1c(Cc2ccccc2S(=O)(=O)N(C)c2ccccc2)c2c(n1CC(=O)O)CC(C)(C)CC2=O] | I-49 |
| 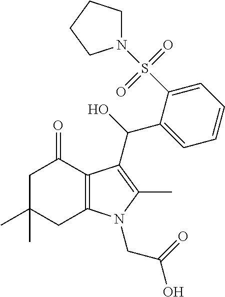 [SMILES: Cc1c(C(O)c2ccccc2S(=O)(=O)N2CCCC2)c2c(n1CC(=O)O)CC(C)(C)CC2=O] | I-50 |
| 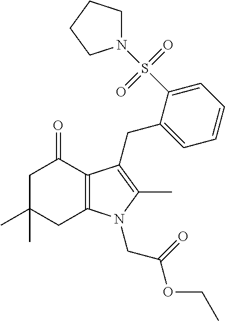 [SMILES: CCOC(=O)Cn1c(C)c(Cc2ccccc2S(=O)(=O)N2CCCC2)c2c1CC(C)(C)CC2=O] | I-51 |
| 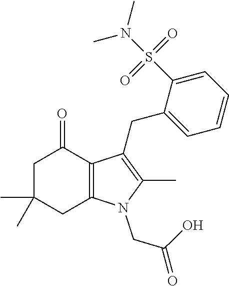 [SMILES: Cc1c(Cc2ccccc2S(=O)(=O)N(C)C)c2c(n1CC(=O)O)CC(C)(C)CC2=O] | I-52 |
| 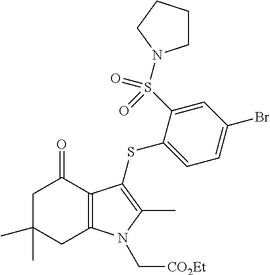 [SMILES: CCOC(=O)Cn1c(C)c(Sc2ccc(Br)cc2S(=O)(=O)N2CCCC2)c2c1CC(C)(C)CC2=O] | I-53 |
| 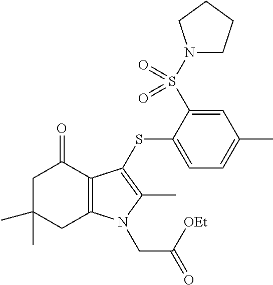 | I-54 |
| 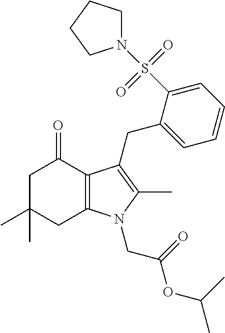 [SMILES: Cc1c(Cc2ccccc2S(=O)(=O)N2CCCC2)c2c(n1CC(=O)OC(C)C)CC(C)(C)CC2=O] | I-55 |
| 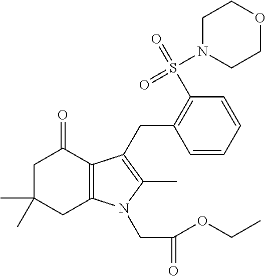 [SMILES: CCOC(=O)Cn1c(C)c(Cc2ccccc2S(=O)(=O)N2CCOCC2)c2c1CC(C)(C)CC2=O] | I-56 |
| 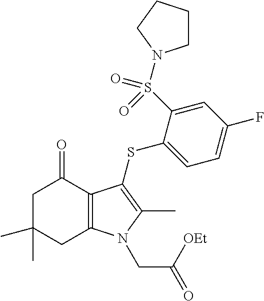 [SMILES: CCOC(=O)Cn1c(C)c(Sc2ccc(F)cc2S(=O)(=O)N2CCCC2)c2c1CC(C)(C)CC2=O] | I-57 |
| 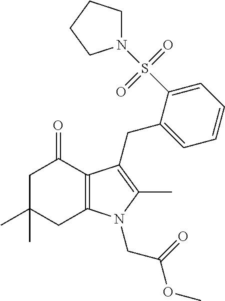 [SMILES: COC(=O)Cn1c(C)c(Cc2ccccc2S(=O)(=O)N2CCCC2)c2c1CC(C)(C)CC2=O] | I-58 |
| 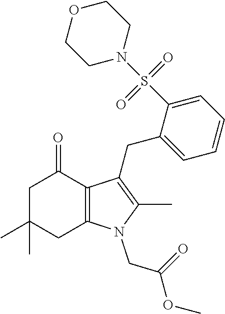 [SMILES: COC(=O)Cn1c(C)c(Cc2ccccc2S(=O)(=O)N2CCOCC2)c2c1CC(C)(C)CC2=O] | I-59 |
| 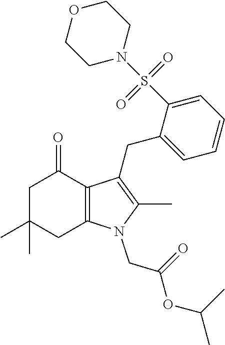 [SMILES: Cc1c(Cc2ccccc2S(=O)(=O)N2CCOCC2)c2c(n1CC(=O)OC(C)C)CC(C)(C)CC2=O] | I-60 |
| 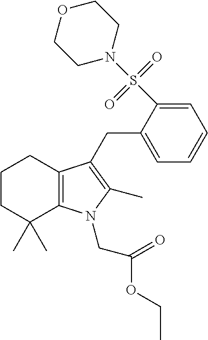 [SMILES: CCOC(=O)Cn1c(C)c(Cc2ccccc2S(=O)(=O)N2CCOCC2)c2c1C(C)(C)CCC2] | I-61 |
| 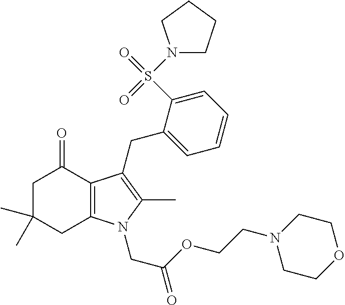 [SMILES: Cc1c(Cc2ccccc2S(=O)(=O)N2CCCC2)c2c(n1CC(=O)OCCN1CCOCC1)CC(C)(C)CC2=O] | I-62 |
| 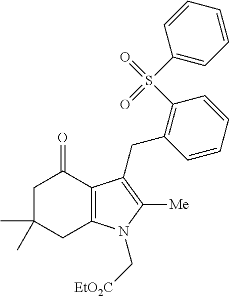 [SMILES: CCOC(=O)Cn1c(C)c(Cc2ccccc2S(=O)(=O)c2ccccc2)c2c1CC(C)(C)CC2=O] | I-63 |
| 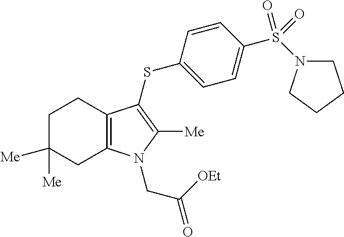 [SMILES: CCOC(=O)Cn1c(C)c(Sc2ccc(S(=O)(=O)N3CCCC3)cc2)c2c1CC(C)(C)CC2] | I-64 |
| 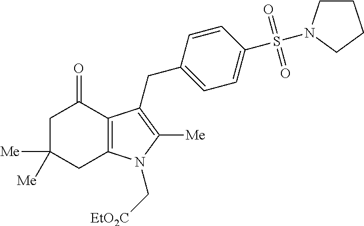 [SMILES: CCOC(=O)Cn1c(C)c(Cc2ccc(S(=O)(=O)N3CCCC3)cc2)c2c1CC(C)(C)CC2=O] | I-65 |
| 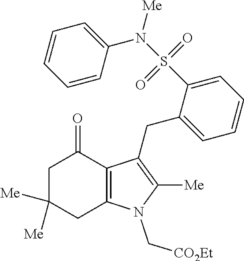 [SMILES: CCOC(=O)Cn1c(C)c(Cc2ccccc2S(=O)(=O)N(C)c2ccccc2)c2c1CC(C)(C)CC2=O] | I-66 |
| 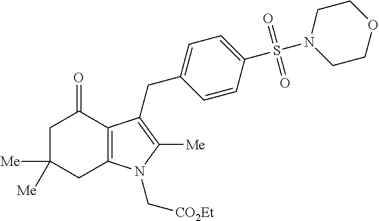 [SMILES: CCOC(=O)Cn1c(C)c(Cc2ccc(S(=O)(=O)N3CCOCC3)cc2)c2c1CC(C)(C)CC2=O] | I-67 |
| 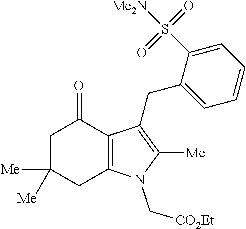 [SMILES: CCOC(=O)Cn1c(C)c(Cc2ccccc2S(=O)(=O)N(C)C)c2c1CC(C)(C)CC2=O] | I-68 |
| 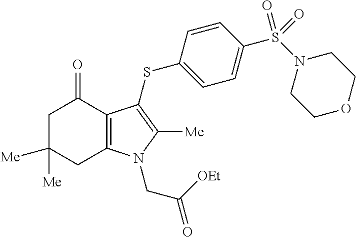 [SMILES: CCOC(=O)Cn1c(C)c(Sc2ccc(S(=O)(=O)N3CCOCC3)cc2)c2c1CC(C)(C)CC2=O] | I-69 |
| 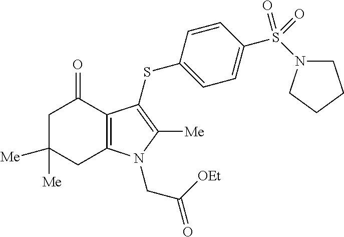 [SMILES: CCOC(=O)Cn1c(C)c(Sc2ccc(S(=O)(=O)N3CCCC3)cc2)c2c1CC(C)(C)CC2=O] | I-70 |
| 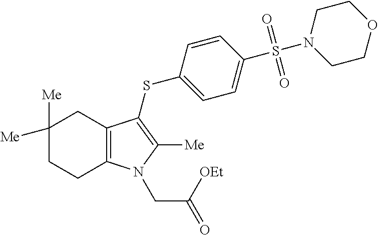 [SMILES: CCOC(=O)Cn1c(C)c(Sc2ccc(S(=O)(=O)N3CCOCC3)cc2)c2c1CCC(C)(C)C2] | I-71 |
| 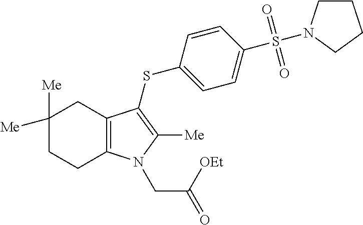 [SMILES: CCOC(=O)Cn1c(C)c(Sc2ccc(S(=O)(=O)N3CCCC3)cc2)c2c1CCC(C)(C)C2] | I-72 |
| 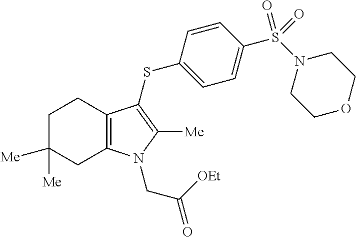 [SMILES: CCOC(=O)Cn1c(C)c(Sc2ccc(S(=O)(=O)N3CCOCC3)cc2)c2c1CC(C)(C)CC2] | I-73 |
| 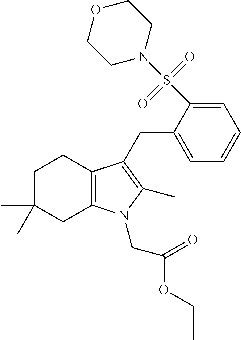 [SMILES: CCOC(=O)Cn1c(C)c(Cc2ccccc2S(=O)(=O)N2CCOCC2)c2c1CC(C)(C)CC2] | I-74 |
| 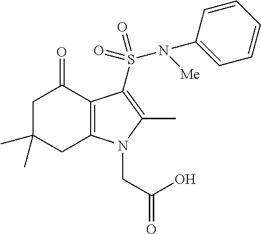 [SMILES: Cc1c(S(=O)(=O)N(C)c2ccccc2)c2c(n1CC(=O)O)CC(C)(C)CC2=O] | I-75 |
| 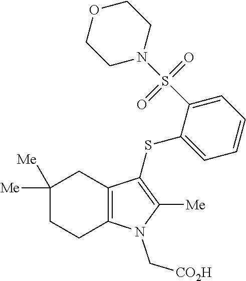 [SMILES: Cc1c(Sc2ccccc2S(=O)(=O)N2CCOCC2)c2c(n1CC(=O)O)CCC(C)(C)C2] | I-76 |
| 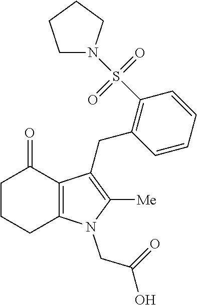 [SMILES: Cc1c(Cc2ccccc2S(=O)(=O)N2CCCC2)c2c(n1CC(=O)O)CCCC2=O] | I-77 |
| 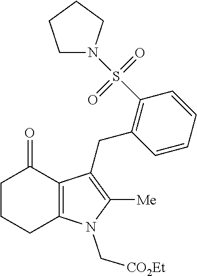 [SMILES: CCOC(=O)Cn1c(C)c(Cc2ccccc2S(=O)(=O)N2CCCC2)c2c1CCCC2=O] | I-78 |
| 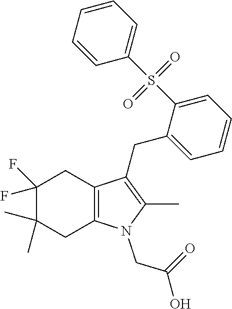 [SMILES: Cc1c(Cc2ccccc2S(=O)(=O)c2ccccc2)c2c(n1CC(=O)O)CC(C)(C)C(F)(F)C2] | I-79 |
| 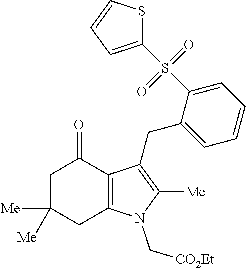 [SMILES: CCOC(=O)Cn1c(C)c(Cc2ccccc2S(=O)(=O)c2cccs2)c2c1CC(C)(C)CC2=O] | I-80 |
| 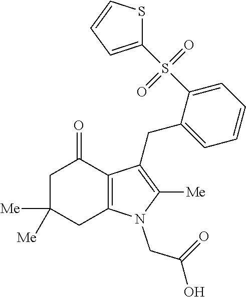 [SMILES: Cc1c(Cc2ccccc2S(=O)(=O)c2cccs2)c2c(n1CC(=O)O)CC(C)(C)CC2=O] | I-81 |
| 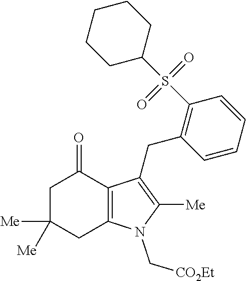 [SMILES: CCOC(=O)Cn1c(C)c(Cc2ccccc2S(=O)(=O)C2CCCCC2)c2c1CC(C)(C)CC2=O] | I-83 |
| 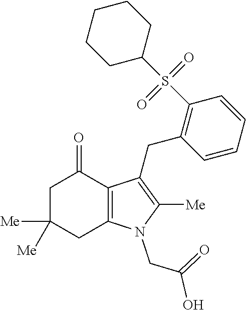 [SMILES: Cc1c(Cc2ccccc2S(=O)(=O)C2CCCCC2)c2c(n1CC(=O)O)CC(C)(C)CC2=O] | I-82 |
| 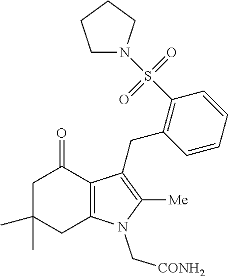 [SMILES: Cc1c(Cc2ccccc2S(=O)(=O)N2CCCC2)c2c(n1CC(N)=O)CC(C)(C)CC2=O] | I-84 |
| 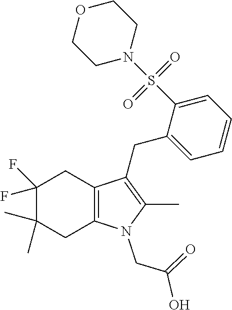 [SMILES: Cc1c(Cc2ccccc2S(=O)(=O)N2CCOCC2)c2c(n1CC(=O)O)CC(C)(C)C(F)(F)C2] | I-85 |
| 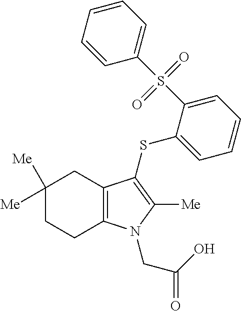 [SMILES: Cc1c(Sc2ccccc2S(=O)(=O)c2ccccc2)c2c(n1CC(=O)O)CCC(C)(C)C2] | I-86 |
| 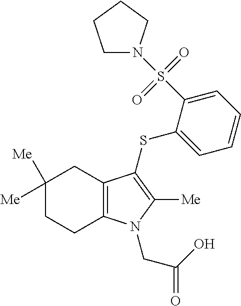 [SMILES: Cc1c(Sc2ccccc2S(=O)(=O)N2CCCC2)c2c(n1CC(=O)O)CCC(C)(C)C2] | I-87 |
| 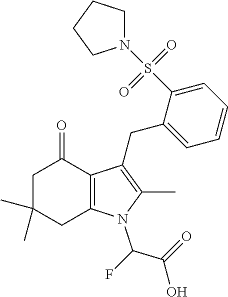 [SMILES: Cc1c(Cc2ccccc2S(=O)(=O)N2CCCC2)c2c(n1C(F)C(=O)O)CC(C)(C)CC2=O] | I-92 |
| 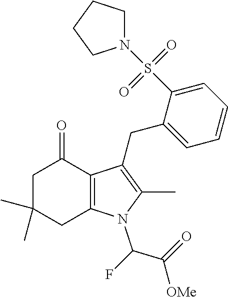 [SMILES: COC(=O)C(F)n1c(C)c(Cc2ccccc2S(=O)(=O)N2CCCC2)c2c1CC(C)(C)CC2=O] | I-93 |
| 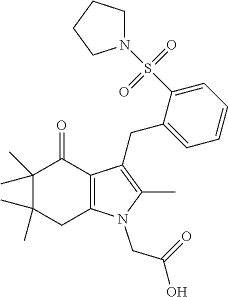 [SMILES: Cc1c(Cc2ccccc2S(=O)(=O)N2CCCC2)c2c(n1CC(=O)O)CC(C)(C)C(C)(C)C2=O] | I-95 |
| 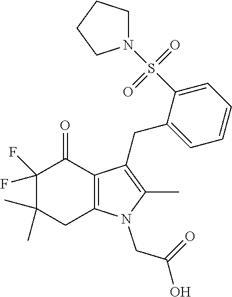 [SMILES: Cc1c(Cc2ccccc2S(=O)(=O)N2CCCC2)c2c(n1CC(=O)O)CC(C)(C)C(F)(F)C2=O] | I-96 |
| 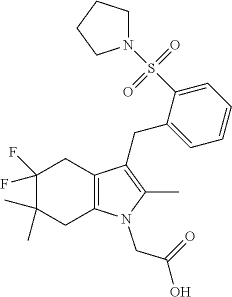 [SMILES: Cc1c(Cc2ccccc2S(=O)(=O)N2CCCC2)c2c(n1CC(=O)O)CC(C)(C)C(F)(F)C2] | I-97 |
| 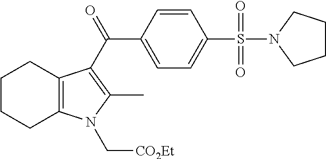 [SMILES: CCOC(=O)Cn1c(C)c(C(=O)c2ccc(S(=O)(=O)N3CCCC3)cc2)c2c1CCCC2] | I-99 |
| 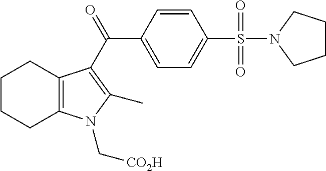 [SMILES: Cc1c(C(=O)c2ccc(S(=O)(=O)N3CCCC3)cc2)c2c(n1CC(=O)O)CCCC2] | I-100 |
| 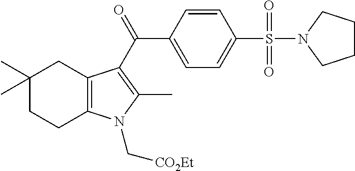 [SMILES: CCOC(=O)Cn1c(C)c(C(=O)c2ccc(S(=O)(=O)N3CCCC3)cc2)c2c1CCC(C)(C)C2] | I-101 |
| 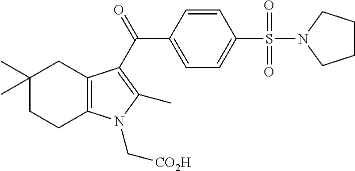 [SMILES: Cc1c(C(=O)c2ccc(S(=O)(=O)N3CCCC3)cc2)c2c(n1CC(=O)O)CCC(C)(C)C2] | I-102 |
| 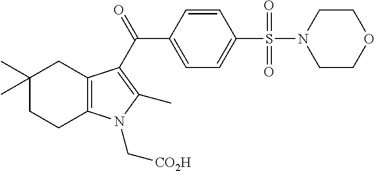 [SMILES: Cc1c(C(=O)c2ccc(S(=O)(=O)N3CCOCC3)cc2)c2c(n1CC(=O)O)CCC(C)(C)C2] | I-103 |
| 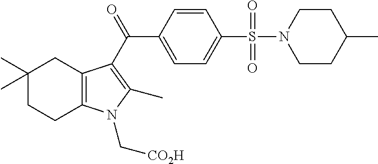 | I-104 |
| 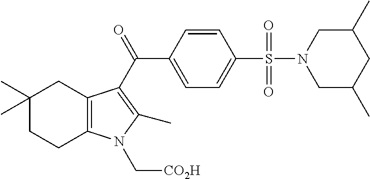 | I-105 |

### Pharmaceutically Acceptable Salts and Pro-Drugs.

The phrase “pharmaceutically acceptable salt,” as used herein, refers to pharmaceutically acceptable organic or inorganic salts of a compound disclosed herein. For use in medicine, the salts of the compounds disclosed hereinwill be pharmaceutically acceptable salts. Other salts may, however, be useful in the preparation of the compounds disclosed herein or of their pharmaceutically acceptable salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

Pharmaceutically acceptable salts of the compounds described herein include those derived from suitable inorganic and organic acids and bases. In some embodiments, the salts can be prepared in situ during the final isolation and purification of the compounds. In other embodiments the salts can be prepared from the free form of the compound in a separate synthetic step.

When the compound disclosed herein is acidic or contains a sufficiently acidic bioisostere, suitable “pharmaceutically acceptable salts” refers to salts prepared form pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particular embodiments include ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine, caffeine, choline, N,N¹-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound disclosed herein is basic or contains a sufficiently basic bioisostere, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particular embodiments include citric, hydrobromic, hydrochloric, maleic, phosphoric, sulfuric and tartaric acids. Other exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts.

The preparation of the pharmaceutically acceptable salts described above and other typical pharmaceutically acceptable salts is more fully described by Berg et al., “Pharmaceutical Salts,” J. Pharm. Sci., 1977:66:1-19, incorporated herein by reference in its entirety.

In addition to the compounds described herein and their pharmaceutically acceptable salts, pharmaceutically acceptable solvates (e.g., hydrates) and co-crystals of these compounds and salts may also be employed in compositions to treat or prevent the herein identified disorders.

As used herein, the term “pharmaceutically acceptable solvate,” is a solvate formed from the association of one or more pharmaceutically acceptable solvent molecules to one of the compounds described herein. As used herein, the term “hydrate” means a compound described herein or a salt thereof that further includes a stoichiometric or non-stoichiometric amount of water bound by non-covalent intermolecular forces. The term solvate includes hydrates (e.g., hemihydrate, monohydrate, dihydrate, trihydrate, tetrahydrate, and the like).

“Pharmaceutically acceptable co-crystals” result when a pharmaceutically active compound crystallizes with another material (e.g. a carboxylic acid, a 4,4′-bipyridine or an excipient) that is also a solid at room temperature. Some pharmaceutically acceptable excipients are described in the next section. Other pharmaceutically acceptable substances that can be used to form co-crystals are exemplified by the GRAS (Generally regarded as safe) list of the US FDA.

In addition to the compounds described herein, pharmaceutically acceptable pro-drugs of these compounds may also be employed in compositions to treat or prevent the herein identified disorders.

A “pharmaceutically acceptable pro-drug” includes any pharmaceutically acceptable ester, salt of an ester or other derivative or salt thereof of a compound described herein which, upon administration to a recipient, is capable of providing, either directly or indirectly, a compound described herein. Particularly favored pro-drugs are those that increase the bioavailability of the compounds when such compounds are administered to a patient (e.g., by allowing an orally administered compound to be more readily absorbed into the blood) or which enhance delivery of the parent compound to a biological compartment (e.g., the brain or lymphatic system) relative to the parent species. The term “pro-drug” encompasses a derivative of a compound that can hydrolyze, oxidize, or otherwise react under biological conditions (in vitro or in vivo) to provide a compound described herein. Examples of pro-drugs include, but are not limited to, analogs or derivatives of compounds disclosed herein that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of pro-drugs include derivatives of compounds that comprise —NO, —NO₂, —ONO, or —ONO2 moieties. Pro-drugs can typically be prepared using well-known methods, such as those described by BURGER'S MEDICINAL CHEMISTRY AND DRUG DISCOVERY (1995) 172-178, 949-982 (Manfred E. Wolff ed., 5th Ed).

### Pharmaceutical Compositions and Methods of Administration

### Compositions of the Invention

The compounds herein disclosed, and their pharmaceutically acceptable salts, solvates, co-crystals and pro-drugs thereof may be formulated as pharmaceutical compositions or “formulations”.

A typical formulation is prepared by mixing a compound disclosed herein, or a pharmaceutically acceptable salt, solvate, co-crystal or pro-drug thereof, and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which the compound disclosed herein is being formulated. Solvents are generally selected based on solvents recognized by persons skilled in the art as safe (GRAS-Generally Regarded as Safe) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations may also include other types of excipients such as one or more buffers, stabilizing agents, antiadherents, surfactants, wetting agents, lubricating agents, emulsifiers, binders, suspending agents, disintegrants, fillers, sorbents, coatings (e.g. enteric or slow release) preservatives, antioxidants, opaquing agents, glidants, processing aids, colorants, sweeteners, perfuming agents, flavoring agents and other known additives to provide an elegant presentation of the drug (i.e., a compound disclosed herein or pharmaceutical composition thereof) or aid in the manufacturing of the pharmaceutical product (i.e., medicament).

The formulations may be prepared using conventional dissolution and mixing procedures. For example, the bulk drug substance (i.e., compound having disclosed herein, a pharmaceutically acceptable salt, solvate, co-crystal or pro-drug thereof, or a stabilized form of the compound, such as a complex with a cyclodextrin derivative or other known complexation agent) is dissolved in a suitable solvent in the presence of one or more of the excipients described above. A compound having the desired degree of purity is optionally mixed with pharmaceutically acceptable diluents, carriers, excipients or stabilizers, in the form of a lyophilized formulation, milled powder, or an aqueous solution. Formulation may be conducted by mixing at ambient temperature at the appropriate pH, and at the desired degree of purity, with physiologically acceptable carriers. The pH of the formulation depends mainly on the particular use and the concentration of compound, but may range from about 3 to about 8. When the agent described herein is a solid amorphous dispersion formed by a solvent process, additives may be added directly to the spray-drying solution when forming the mixture such as the additive is dissolved or suspended in the solution as a slurry which can then be spray dried. Alternatively, the additives may be added following spray-drying process to aid in the forming of the final formulated product.

The compound disclosed herein or a pharmaceutically acceptable salt, solvate, co-crystal or pro-drug thereof is typically formulated into pharmaceutical dosage forms to provide an easily controllable dosage of the drug and to enable patient compliance with the prescribed regimen. Pharmaceutical formulations of compounds disclosed herein, or a pharmaceutically acceptable salt, solvate, co-crystal or pro-drug thereof, may be prepared for various routes and types of administration. Various dosage forms may exist for the same compound, since different medical conditions may warrant different routes of administration.

The compounds disclosed herein may be administered at a dose from about 200 to 1400 mg once or twice a day. In particular, the amount of compound is approximately 300 to 1300 mg, 400 to 1200 mg, 500 to 1100 mg, 600 to 1000 mg, 700 to 900 mg, 750 to 850 mg, 200 to 500 mg, 300 to 600 mg, 400 to 700 mg, 500 to 800 mg, 600 to 900 mg, 700 to 1000 mg, 800 to 1100 mg, 900 to 1200 mg, 1000 to 1300 mg or 1100 to 1400 mg. In particular, the amount of compound is approximately 300 mg, 600 mg, 1000 mg or 2000 mg.

The amount of active ingredient that may be combined with the carrier material to produce a single dosage form will vary depending upon the subject treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight: weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur. As a general proposition, the initial pharmaceutically effective amount of the inhibitor administered will be in the range of about 0.01-100 mg/kg per dose, namely about 0.1 to 20 mg/kg of patient body weight per day, with the typical initial range of compound used being 0.3 to 15 mg/kg/day.

The term “therapeutically effective amount” as used herein means that amount of active compound or pharmaceutical agent that elicits the biological or medicinal response in a tissue, system, animal or human that is being sought by a researcher, veterinarian, medical doctor or other clinician. The therapeutically or pharmaceutically effective amount” of the compound to be administered will be governed by such considerations, and is the minimum amount necessary to ameliorate, cure or treat the disease or disorder or one or more of its symptoms.

The pharmaceutical compositions disclosed herein will be formulated, dosed, and administered in a fashion, i.e., amounts, concentrations, schedules, course, vehicles, and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners, such as the age, weight, and response of the individual patient.

The term “prophylactically effective amount” refers to an amount effective in preventing or substantially lessening the chances of acquiring a disease or disorder or in reducing the severity of the disease or disorder or one or more of its symptoms before it is acquired or before the symptoms develop. Roughly, prophylactic measures are divided between primary prophylaxis (to prevent the development of a disease) and secondary prophylaxis (whereby the disease has already developed and the patient is protected against worsening of this process).

Acceptable diluents, carriers, excipients, and stabilizers are those that are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). The active pharmaceutical ingredients may also be entrapped in microcapsules prepared, for example, by coacervation techniques or by interfacial polymerization, e.g., hydroxymethylcellulose or gelatin-microcapsules and poly-(methylmethacylate) microcapsules, respectively; in colloidal drug delivery systems (for example, liposomes, albumin microspheres, microemulsions, nano-particles and nanocapsules) or in macroemulsions. Such techniques are disclosed in Remington's: The Science and Practice of Pharmacy, 21^{st} Edition, University of the Sciences in Philadelphia, Eds., 2005 (hereafter “Remington's”).

“Controlled drug delivery systems” supply the drug to the body in a manner precisely controlled to suit the drug and the conditions being treated. The primary aim is to achieve a therapeutic drug concentration at the site of action for the desired duration of time. The term “controlled release” is often used to refer to a variety of methods that modify release of drug from a dosage form. This term includes preparations labeled as “extended release”, “delayed release”, “modified release” or “sustained release”. In general, one can provide for controlled release of the agents described herein through the use of a wide variety of polymeric carriers and controlled release systems including erodible and non-erodible matrices, osmotic control devices, various reservoir devices, enteric coatings and multiparticulate control devices.

“Sustained-release preparations” are the most common applications of controlled release. Suitable examples of sustained-release preparations include semipermeable matrices of solid hydrophobic polymers containing the compound, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers, and poly-D-(−)-3-hydroxybutyric acid.

“Immediate-release preparations” may also be prepared. The objective of these formulations is to get the drug into the bloodstream and to the site of action as rapidly as possible. For instance, for rapid dissolution, most tablets are designed to undergo rapid disintegration to granules and subsequent disaggregation to fine particles. This provides a larger surface area exposed to the dissolution medium, resulting in a faster dissolution rate.

Agents described herein can be incorporated into an erodible or non-erodible polymeric matrix controlled release device. By an erodible matrix is meant aqueous-erodible or water-swellable or aqueous-soluble in the sense of being either erodible or swellable or dissolvable in pure water or requiring the presence of an acid or base to ionize the polymeric matrix sufficiently to cause erosion or dissolution. When contacted with the aqueous environment of use, the erodible polymeric matrix imbibes water and forms an aqueous-swollen gel or matrix that entraps the agent described herein. The aqueous-swollen matrix gradually erodes, swells, disintegrates or dissolves in the environment of use, thereby controlling the release of a compound described herein to the environment of use. One ingredient of this water-swollen matrix is the water-swellable, erodible, or soluble polymer, which may generally be described as an osmopolymer, hydrogel or water-swellable polymer. Such polymers may be linear, branched, or cross linked. The polymers may be homopolymers or copolymers. In certain embodiments, they may be synthetic polymers derived from vinyl, acrylate, methacrylate, urethane, ester and oxide monomers. In other embodiments, they can be derivatives of naturally occurring polymers such as polysaccharides (e.g. chitin, chitosan, dextran and pullulan; gum agar, gum arabic, gum karaya, locust bean gum, gum tragacanth, carrageenans, gum ghatti, guar gum, xanthan gum and scleroglucan), starches (e.g. dextrin and maltodextrin), hydrophilic colloids (e.g. pectin), phosphatides (e.g. lecithin), alginates (e.g. ammonium alginate, sodium, potassium or calcium alginate, propylene glycol alginate), gelatin, collagen, and cellulosics. Cellulosics are cellulose polymer that has been modified by reaction of at least a portion of the hydroxyl groups on the saccharide repeat units with a compound to form an ester-linked or an ether-linked substituent. For example, the cellulosic ethyl cellulose has an ether linked ethyl substituent attached to the saccharide repeat unit, while the cellulosic cellulose acetate has an ester linked acetate substituent. In certain embodiments, the cellulosics for the erodible matrix comprises aqueous-soluble and aqueous-erodible cellulosics can include, for example, ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC). In certain embodiments, the cellulosics comprises various grades of low viscosity (MW less than or equal to 50,000 daltons, for example, the Dow Methocel™ series E5, E15LV, E50LV and K100LY) and high viscosity (MW greater than 50,000 daltons, for example, E4MCR, E10MCR, K4M, K15M and K100M and the Methocel™ K series) HPMC. Other commercially available types of HPMC include the Shin Etsu Metolose 90SH series.

Other materials useful as the erodible matrix material include, but are not limited to, pullulan, polyvinyl pyrrolidone, polyvinyl alcohol, polyvinyl acetate, glycerol fatty acid esters, polyacrylamide, polyacrylic acid, copolymers of ethacrylic acid or methacrylic acid (EUDRAGITO, Rohm America, Inc., Piscataway, N.J.) and other acrylic acid derivatives such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

Alternatively, the agents of the present invention may be administered by or incorporated into a non-erodible matrix device. In such devices, an agent described herein is distributed in an inert matrix. The agent is released by diffusion through the inert matrix. Examples of materials suitable for the inert matrix include insoluble plastics (e.g methyl acrylate-methyl methacrylate copolymers, polyvinyl chloride, polyethylene), hydrophilic polymers (e.g. ethyl cellulose, cellulose acetate, cross linked polyvinylpyrrolidone (also known as crospovidone)), and fatty compounds (e.g. carnauba wax, microcrystalline wax, and triglycerides). Such devices are described further in Remington: The Science and Practice of Pharmacy, 20th edition (2000).

As noted above, the agents described herein may also be incorporated into an osmotic control device. Such devices generally include a core containing one or more agents as described herein and a water-permeable, non-dissolving and non-eroding coating surrounding the core which controls the influx of water into the core from an aqueous environment of use so as to cause drug release by extrusion of some or the entire core to the environment of use. In certain embodiments, the coating is polymeric, aqueous-permeable, and has at least one delivery port. The core of the osmotic device optionally includes an osmotic agent which acts to imbibe water from the surrounding environment via such a semi-permeable membrane. The osmotic agent contained in the core of this device may be an aqueous-swellable hydrophilic polymer or it may be an osmogen, also known as an osmagent. Pressure is generated within the device which forces the agent(s) out of the device via an orifice (of a size designed to minimize solute diffusion while preventing the build-up of a hydrostatic pressure head). Non-limiting examples of osmotic control devices are disclosed in U.S. patent application Ser. No. 09/495,061.

The amount of water-swellable hydrophilic polymers present in the core may range from about 5 to about 80 wt % (including for example, 10 to 50 wt %). Non limiting examples of core materials include hydrophilic vinyl and acrylic polymers, polysaccharides such as calcium alginate, polyethylene oxide (PEO), polyethylene glycol (PEG), polypropylene glycol (PPG), poly (2-hydroxyethyl methacrylate), poly (acrylic) acid, poly (methacrylic) acid, polyvinylpyrrolidone (PVP) and cross linked PVP, polyvinyl alcohol (PVA), PVA/PVP copolymers and PVA/PVP copolymers with hydrophobic monomers such as methyl methacrylate, vinyl acetate, and the like, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl cellulose (CEC), sodium alginate, polycarbophil, gelatin, xanthan gum, and sodium starch glycolate. Other materials include hydrogels comprising interpenetrating networks of polymers that may be formed by addition or by condensation polymerization, the components of which may comprise hydrophilic and hydrophobic monomers such as those just mentioned. Water-swellable hydrophilic polymers include but are not limited to PEO, PEG, PVP, sodium croscarmellose, HPMC, sodium starch glycolate, polyacrylic acid and cross linked versions or mixtures thereof.

The core may also include an osmogen (or osmagent). The amount of osmogen present in the core may range from about 2 to about 70 wt % (including, for example, from 10 to 50 wt %). Typical classes of suitable osmogens are water-soluble organic acids, salts and sugars that are capable of imbibing water to thereby effect an osmotic pressure gradient across the barrier of the surrounding coating. Typical useful osmogens include but are not limited to magnesium sulfate, magnesium chloride, calcium chloride, sodium chloride, lithium chloride, potassium sulfate, sodium carbonate, sodium sulfite, lithium sulfate, potassium chloride, sodium sulfate, mannitol, xylitol, urea, sorbitol, inositol, raffinose, sucrose, glucose, fructose, lactose, citric acid, succinic acid, tartaric acid, and mixtures thereof. In certain embodiments, the osmogen is glucose, lactose, sucrose, mannitol, xylitol, sodium chloride, including combinations thereof.

The rate of drug delivery is controlled by such factors as the permeability and thickness of the coating, the osmotic pressure of the drug-containing layer, the degree of hydrophilicity of the hydrogel layer, and the surface area of the device. Those skilled in the art will appreciate that increasing the thickness of the coating will reduce the release rate, while any of the following will increase the release rate: increasing the permeability of the coating; increasing the hydrophilicity of the hydrogel layer; increasing the osmotic pressure of the drug-containing layer; or increasing the device's surface area.

In certain embodiments, entrainment of particles of agents described herein in the extruding fluid during operation of such osmotic device is desirable. For the particles to be well entrained, the agent drug form is dispersed in the fluid before the particles have an opportunity to settle in the tablet core. One means of accomplishing this is by adding a disintegrant that serves to break up the compressed core into its particulate components. Non-limiting examples of standard disintegrants include materials such as sodium starch glycolate (e.g., Explotab™ CLV), microcrystalline cellulose (e.g., Avicel™), microcrystalline silicified cellulose (e.g., ProSoIv™) and croscarmellose sodium (e.g., Ac-Di-Sol™), and other disintegrants known to those skilled in the art. Depending upon the particular formulation, some disintegrants work better than others. Several disintegrants tend to form gels as they swell with water, thus hindering drug delivery from the device. Non-gelling, non-swelling disintegrants provide a more rapid dispersion of the drug particles within the core as water enters the core. In certain embodiments, non-gelling, non-swelling disintegrants are resins, for example, ion-exchange resins. In one embodiment, the resin is Amberlite™ IRP 88 (available from Rohm and Haas, Philadelphia, Pa.). When used, the disintegrant is present in amounts ranging from about 1-25% of the core agent.

Another example of an osmotic device is an osmotic capsule. The capsule shell or portion of the capsule shell can be semipermeable. The capsule can be filled either by a powder or liquid consisting of an agent described herein, excipients that imbibe water to provide osmotic potential, and/or a water-swellable polymer, or optionally solubilizing excipients. The capsule core can also be made such that it has a bilayer or multilayer agent analogous to the bilayer, trilayer or concentric geometries described above.

Another class of osmotic device useful in this invention comprises coated swellable tablets, for example, as described in EP378404. Coated swellable tablets comprise a tablet core comprising an agent described herein and a swelling material, preferably a hydrophilic polymer, coated with a membrane, which contains holes, or pores through which, in the aqueous use environment, the hydrophilic polymer can extrude and carry out the agent. Alternatively, the membrane may contain polymeric or low molecular weight water-soluble porosigens. Porosigens dissolve in the aqueous use environment, providing pores through which the hydrophilic polymer and agent may extrude. Examples of porosigens are water-soluble polymers such as HPMC, PEG, and low molecular weight compounds such as glycerol, sucrose, glucose, and sodium chloride. In addition, pores may be formed in the coating by drilling holes in the coating using a laser or other mechanical means. In this class of osmotic devices, the membrane material may comprise any film-forming polymer, including polymers which are water permeable or impermeable, providing that the membrane deposited on the tablet core is porous or contains water-soluble porosigens or possesses a macroscopic hole for water ingress and drug release. Embodiments of this class of sustained release devices may also be multilayered, as described, for example, in EP378404.

When an agent described herein is a liquid or oil, such as a lipid vehicle formulation, for example as described in WO05/011634, the osmotic controlled-release device may comprise a soft-gel or gelatin capsule formed with a composite wall and comprising the liquid formulation where the wall comprises a barrier layer formed over the external surface of the capsule, an expandable layer formed over the barrier layer, and a semipermeable layer formed over the expandable layer. A delivery port connects the liquid formulation with the aqueous use environment. Such devices are described, for example, in U.S. Pat. No. 6,419,952, U.S. Pat. No. 6,342,249, U.S. Pat. No. 5,324,280, U.S. Pat. No. 4,672,850, U.S. Pat. No. 4,627,850, U.S. Pat. No. 4,203,440, and U.S. Pat. No. 3,995,631.

As further noted above, the agents described herein may be provided in the form of microparticulates, generally ranging in size from about 10 μm to about 2 mm (including, for example, from about 100 μm to 1 mm in diameter). Such multiparticulates may be packaged, for example, in a capsule such as a gelatin capsule or a capsule formed from an aqueous-soluble polymer such as HPMCAS, HPMC or starch; dosed as a suspension or slurry in a liquid; or they may be formed into a tablet, caplet, or pill by compression or other processes known in the art. Such multiparticulates may be made by any known process, such as wet- and dry-granulation processes, extrusion/spheronization, roller-compaction, melt-congealing, or by spray-coating seed cores. For example, in wet- and dry-granulation processes, the agent described herein and optional excipients may be granulated to form multiparticulates of the desired size.

The agents can be incorporated into microemulsions, which generally are thermodynamically stable, isotropically clear dispersions of two immiscible liquids, such as oil and water, stabilized by an interfacial film of surfactant molecules (Encyclopedia of Pharmaceutical Technology (New York: Marcel Dekker, 1992), volume 9). For the preparation of microemulsions, surfactant (emulsifier), co-surfactant (co-emulsifier), an oil phase and a water phase are necessary. Suitable surfactants include any surfactants that are useful in the preparation of emulsions, e.g., emulsifiers that are typically used in the preparation of creams. The co-surfactant (or “co-emulsifier”) is generally selected from the group of polyglycerol derivatives, glycerol derivatives and fatty alcohols. Preferred emulsifier/co-emulsifier combinations are generally although not necessarily selected from the group consisting of: glyceryl monostearate and polyoxyethylene stearate; polyethylene glycol and ethylene glycol palmitostearate; and caprilic and capric triglycerides and oleoyl macrogolglycerides. The water phase includes not only water but also, typically, buffers, glucose, propylene glycol, polyethylene glycols, preferably lower molecular weight polyethylene glycols (e.g., PEG 300 and PEG 400), and/or glycerol, and the like, while the oil phase will generally comprise, for example, fatty acid esters, modified vegetable oils, silicone oils, mixtures of mono- di- and triglycerides, mono- and di-esters of PEG (e.g., oleoyl macrogol glycerides), etc.

The compounds described herein can be incorporated into pharmaceutically-acceptable nanoparticle, nanosphere, and nanocapsule formulations (Delie and Blanco-Prieto 2005 Molecule 10:65-80). Nanocapsules can generally entrap compounds in a stable and reproducible way (Henry-Michelland et al., 1987; Quintanar-Guerrero et al., 1998; Douglas et al., 1987). To avoid side effects due to intracellular polymeric overloading, ultrafine particles (sized around 0.1 μm) can be designed using polymers able to be degraded in vivo (e.g. biodegradable polyalkyl-cyanoacrylate nanoparticles). Such particles are described in the prior art (Couvreur et al, 1980; 1988; zur Muhlen et al., 1998; Zambaux et al. 1998; Pinto-Alphandry et al., 1995 and U.S. Pat. No. 5,145,684).

Implantable devices coated with a compound of this invention are another embodiment of the present invention. The compounds may also be coated on implantable medical devices, such as beads, or co-formulated with a polymer or other molecule, to provide a “drug depot”, thus permitting the drug to be released over a longer time period than administration of an aqueous solution of the drug. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition.

The formulations include those suitable for the administration routes detailed herein. The formulations may conveniently be presented in unit dosage form and may be prepared by any of the methods well known in the art of pharmacy. Techniques and formulations generally are found in Remington's. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

The terms “administer”, “administering” or “administration” in reference to a compound, composition or formulation disclosed herein means introducing the compound into the system of the animal in need of treatment. When a compound disclosed herein is provided in combination with one or more other active agents, “administration” and its variants are each understood to include concurrent and/or sequential introduction of the compound and the other active agents.

The compositions described herein may be administered systemically or locally, e.g.: orally (e.g. using capsules, powders, solutions, suspensions, tablets, sublingual tablets and the like), by inhalation (e.g. with an aerosol, gas, inhaler, nebulizer or the like), to the ear (e.g. using ear drops), topically (e.g. using creams, gels, liniments, lotions, ointments, pastes, transdermal patches, etc), rectally (e.g. using enemas or suppositories), nasally, buccally, vaginally (e.g. using douches, intrauterine devices, vaginal suppositories, vaginal rings or tablets, etc), via an implanted reservoir or the like, or parenterally depending on the severity and type of the disease being treated. The term “parenteral” as used herein includes, but is not limited to, subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

The pharmaceutical compositions described herein may be orally administered in any orally acceptable dosage form including, but not limited to, capsules, tablets, aqueous suspensions or solutions. Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. Tablets may be uncoated or may be coated by known techniques including microencapsulation to mask an unpleasant taste or to delay disintegration and adsorption in the gastrointestinal tract and thereby provide a sustained action over a longer period. For example, a time delay material such as glyceryl monostearate or glyceryl distearate alone or with a wax may be employed. A water soluble taste masking material such as hydroxypropyl-methylcellulose or hydroxypropyl-cellulose may be employed.

Formulations of a compound disclosed herein that are suitable for oral administration may be prepared as discrete units such as tablets, pills, troches, lozenges, aqueous or oil suspensions, dispersible powders or granules, emulsions, hard or soft capsules, e.g. gelatin capsules, syrups or elixirs. Formulations of a compound intended for oral use may be prepared according to any method known to the art for the manufacture of pharmaceutical compositions.

Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

The active compounds can also be in microencapsulated form with one or more excipients as noted above.

When aqueous suspensions are required for oral use, the active ingredient is combined with emulsifying and suspending agents. If desired, certain sweetening and/or flavoring agents may be added. Syrups and elixirs may be formulated with sweetening agents, for example glycerol, propylene glycol, sorbitol or sucrose. Such formulations may also contain a demulcent, a preservative, flavoring and coloring agents and antioxidant.

Sterile injectable forms of the compositions described herein (e.g. for parenteral administration) may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of injectable formulations.

Oily suspensions may be formulated by suspending the compound disclosed herein in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an antioxidant such as butylated hydroxyanisol or alpha-tocopherol.

Aqueous suspensions of compounds disclosed herein contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound described herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsulated matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

The injectable solutions or microemulsions may be introduced into a patient's bloodstream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

Compositions for rectal or vaginal administration are preferably suppositories which can be prepared by mixing the compounds described herein with suitable non-irritating excipients or carriers such as cocoa butter, beeswax, polyethylene glycol or a suppository wax which are solid at ambient temperature but liquid at body temperature and therefore melt in the rectum or vaginal cavity and release the active compound. Other formulations suitable for vaginal administration may be presented as pessaries, tampons, creams, gels, pastes, foams or sprays.

The pharmaceutical compositions described herein may also be administered topically, especially when the target of treatment includes areas or organs readily accessible by topical application, including diseases of the eye, the ear, the skin, or the lower intestinal tract. Suitable topical formulations are readily prepared for each of these areas or organs.

Dosage forms for topical or transdermal administration of a compound described herein include ointments, pastes, creams, lotions, gels, powders, solutions, sprays, inhalants or patches. The active component is admixed under sterile conditions with a pharmaceutically acceptable carrier and any needed preservatives or buffers as may be required. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel. Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds disclosed herein include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water.

Alternatively, the active ingredients may be formulated in a cream with an oil-in-water cream base. If desired, the aqueous phase of the cream base may include a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulfoxide and related analogs.

The oily phase of emulsions prepared using compounds disclosed herein may be constituted from known ingredients in a known manner. While the phase may comprise merely an emulsifier (otherwise known as an emulgent), it desirably comprises a mixture of at least one emulsifier with a fat or an oil or with both a fat and an oil. A hydrophilic emulsifier may be included together with a lipophilic emulsifier which acts as a stabilizer. In some embodiments, the emulsifier includes both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. Emulgents and emulsion stabilizers suitable for use in the formulation of compounds having disclosed herein include Tween™-60, Span™-80, cetostearyl alcohol, benzyl alcohol, myristyl alcohol, glyceryl mono-stearate and sodium lauryl sulfate.

The pharmaceutical compositions may also be administered by nasal aerosol or by inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. Formulations suitable for intrapulmonary or nasal administration have a particle size for example in the range of 0.1 to 500 micros (including particles in a range between 0.1 and 500 microns in increments microns such as 0.5, 1, 30, 35 microns, etc) which is administered by rapid inhalation through the nasal passage or by inhalation through the mouth so as to reach the alveolar sacs.

The pharmaceutical composition (or formulation) for use may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

In another aspect, a compound disclosed herein or a pharmaceutically acceptable salt thereof, co-crystal, solvate or pro-drug thereof may be formulated in a veterinary composition comprising a veterinary carrier. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

### DEFINITIONS

As used herein, the terms “subject” and “patient” are used interchangeably. The terms “subject” and “patient” refer to an animal (e.g., a bird such as a chicken, quail or turkey, or a mammal). A “mammal” includes a non-primate (e.g., a cow, pig, horse, sheep, rabbit, guinea pig, rat, cat, dog, and mouse) and a primate (e.g., a monkey, chimpanzee and a human), and in particular a human. In one embodiment, the subject is a non-human animal such as a farm animal (e.g., a horse, cow, pig or sheep), or a pet (e.g., a dog, cat, guinea pig or rabbit). In another embodiment, the subject is a human.

The term “biological sample”, as used herein, refers to an in vitro or ex vivo sample, and includes, without limitation, cell cultures or extracts thereof; biopsied material obtained from a mammal or extracts thereof; blood, saliva, urine, faeces, semen, tears, lymphatic fluid, ocular fluid, vitreous humour, or other body fluids or extracts thereof.

“Treat”, “treating” or “treatment” with regard to a disorder or disease refers to alleviating or abrogating the cause and/or the effects of the disorder or disease. As used herein, the terms “treat”, “treatment” and “treating” refer to the reduction or amelioration of the progression, severity and/or duration of alopecia or acne, or the amelioration of one or more symptoms (preferably, one or more discernible symptoms) of said condition, resulting from the administration of one or more therapies (e.g., one or more therapeutic agents such as a compound or composition disclosed herein). In specific embodiments, the terms “treat”, “treatment” and “treating” refer to the amelioration of at least one measurable physical parameter of alopecia or acne. In other embodiments the terms “treat”, “treatment” and “treating” refer to the inhibition of the progression of alopecia or acne, either physically by, e.g., stabilization of a discernible symptom, physiologically by, e.g., stabilization of a physical parameter, or both.

The term “preventing” as used herein refers to administering a medicament beforehand to forestall or obtund an attack. The person of ordinary skill in the medical art (to which the present method claims are directed) recognizes that the term “prevent” is not an absolute term. In the medical art it is understood to refer to the prophylactic administration of a drug to substantially diminish the likelihood of developing or seriousness of a condition, and this is the sense intended. For example, in the Physician's Desk Reference, a standard text in the field, the term “prevent” occurs hundreds of times. As used herein, the terms “prevent”, “preventing” and “prevention” with regard to a disorder or disease refer to averting the cause and/or effects of a disease or disorder prior to the disease or disorder manifesting itself. The terms “prophylaxis” or “prophylactic use”, as used herein, refer to any medical or public health procedure whose purpose is to prevent, rather than treat or cure a disease. As used herein, the terms “prevent”, “prevention” and “preventing” also refer to the reduction in the risk of acquiring or developing a given condition, or the reduction or inhibition of the recurrence or said condition in a subject who is not ill, but who has been or may be near a person with the disease.

In one embodiment, the methods of the invention are a preventative or “pre-emptive” measure to a patient, preferably a human; having a predisposition to developing alopecia or acne. For example, the compounds described herein may be used to prevent the onset or re-occurrence of an acne, or prevent the onset or re-occurrence of alopecia.

The compounds and pharmaceutical compositions described herein can be used alone or in combination therapy for the treatment or prevention of a disease or disorder mediated, regulated or influenced by, for example, Th2 cells, eosinophils, basophils, platelets, Langerhans cells, dendritic cells or mast cells. They also may be used to aid in the prevention or treatment of a disease or disorder mediated, regulated or influenced by PGD₂ and metabolites thereof, such as 13,14-dihydro-15-keto-PGD₂ and 15-deoxy-A1 2,1′-PGD₂.

Compounds and compositions disclosed herein are also useful for veterinary treatment of companion animals, exotic animals and farm animals, including, without limitation, dogs, cats, mice, rats, hamsters, gerbils, guinea pigs, rabbits, horses, pigs and cattle.

### Combination Therapies

The compounds and pharmaceutical compositions described herein can be used in combination therapy with one or more additional therapeutic agents. For combination treatment with more than one active agent, where the active agents are in separate dosage formulations, the active agents may be administered separately or in conjunction. In addition, the administration of one element may be prior to, concurrent to, or subsequent to the administration of the other agent.

When co-administered with other agents, e.g., when co-administered with another alopecia or acne medication, an “effective amount” of the second agent will depend on the type of drug used. Suitable dosages are known for approved agents and can be adjusted by the skilled artisan according to the condition of the subject, the type of condition(s) being treated and the amount of a compound described herein being used. In cases where no amount is expressly noted, an effective amount should be assumed. For example, compounds described herein can be administered to a subject in a dosage range from between about 0.01 to about 10,000 mg/kg body weight/day, about 0.01 to about 5000 mg/kg body weight/day, about 0.01 to about 3000 mg/kg body weight/day, about 0.01 to about 1000 mg/kg body weight/day, about 0.01 to about 500 mg/kg body weight/day, about 0.01 to about 300 mg/kg body weight/day, about 0.01 to about 100 mg/kg body weight/day.

When “combination therapy” is employed, an effective amount can be achieved using a first amount of a compound disclosed herein or a pharmaceutically acceptable salt, solvate (e.g., hydrate), co-crystal or pro-drug thereof and a second amount of an additional suitable therapeutic agent (e.g. an agent to treat alopecia or acne).

In one embodiment of this invention, the compound disclosed herein and the additional therapeutic agent are each administered in an effective amount (i.e., each in an amount which would be therapeutically effective if administered alone). In another embodiment, the compound disclosed herein and the additional therapeutic agent are each administered in an amount which alone does not provide a therapeutic effect (a sub-therapeutic dose). In yet another embodiment, the compound disclosed herein can be administered in an effective amount, while the additional therapeutic agent is administered in a sub-therapeutic dose. In still another embodiment, the compound disclosed herein can be administered in a sub-therapeutic dose, while the additional therapeutic agent, for example, a suitable cancer-therapeutic agent is administered in an effective amount.

As used herein, the terms “in combination” or “co-administration” can be used interchangeably to refer to the use of more than one therapy (e.g., one or more prophylactic and/or therapeutic agents). The use of the terms does not restrict the order in which therapies (e.g., prophylactic and/or therapeutic agents) are administered to a subject.

Co-administration encompasses administration of the first and second amounts of the compounds in an essentially simultaneous manner, such as in a single pharmaceutical composition, for example, capsule or tablet having a fixed ratio of first and second amounts, or in multiple, separate capsules or tablets for each. In addition, such coadministration also encompasses use of each compound in a sequential manner in either order. When co-administration involves the separate administration of the first amount of a compound having Structural Formulae I and a second amount of an additional therapeutic agent, the compounds are administered sufficiently close in time to have the desired therapeutic effect. For example, the period of time between each administration which can result in the desired therapeutic effect, can range from minutes to hours and can be determined taking into account the properties of each compound such as potency, solubility, bioavailability, plasma half-life and kinetic profile. For example, a compound disclosed herein and the second therapeutic agent can be administered in any order within about 24 hours of each other, within about 16 hours of each other, within about 8 hours of each other, within about 4 hours of each other, within about 1 hour of each other or within about 30 minutes of each other.

More, specifically, a first therapy (e.g., a prophylactic or therapeutic agent such as a compound described herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent such as an anti-cancer agent) to a subject.

Examples of other therapeutic agents that may be combined with a compound of the invention, either administered separately or in the same pharmaceutical compositions, include, but are not limited to:

(1) finasteride, minoxidil, or corticosteroids (corticosteroids, such as beclomethasone, methylprednisolone, betamethasone, prednisone, prenisolone, triamcinolone, dexamethasone, fluticasone, flunisolide and hydrocortisone, and corticosteroid analogs such as budesonide).

(2) clindamycin, erythromycin, benzoyl peroxide, tretinoin, tazarotene, adapalene, azelaic acid, tetracycline, doxycycline, minocycline, erythromycin, or isotretinoin.

### Kits

The compounds and pharmaceutical formulations described herein may be contained in a kit. The kit may include single or multiple doses of two or more agents, each packaged or formulated individually, or single or multiple doses of two or more agents packaged or formulated in combination. Thus, one or more agents can be present in first container, and the kit can optionally include one or more agents in a second container. The container or containers are placed within a package, and the package can optionally include administration or dosage instructions. A kit can include additional components such as syringes or other means for administering the agents as well as diluents or other means for formulation. Thus, the kits can comprise: a) a pharmaceutical composition comprising a compound described herein and a pharmaceutically acceptable carrier, vehicle or diluent; and b) a container or packaging. The kits may optionally comprise instructions describing a method of using the pharmaceutical compositions in one or more of the methods described herein (e.g. preventing or treating one or more of the diseases and disorders described herein). The kit may optionally comprise a second pharmaceutical composition comprising one or more additional agents described herein for cotherapy use, a pharmaceutically acceptable carrier, vehicle or diluent. The pharmaceutical composition comprising the compound described herein and the second pharmaceutical composition contained in the kit may be optionally combined in the same pharmaceutical composition.

A kit includes a container or packaging for containing the pharmaceutical compositions and may also include divided containers such as a divided bottle or a divided foil packet. The container can be, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. It is feasible that more than one container can be used together in a single package to market a single dosage form. For example, tablets may be contained in a bottle which is in turn contained within a box.

An example of a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide written memory aid containing information and/or instructions for the physician, pharmacist or subject regarding when the medication is to be taken. A “daily dose” can be a single tablet or capsule or several tablets or capsules to be taken on a given day. When the kit contains separate compositions, a daily dose of one or more compositions of the kit can consist of one tablet or capsule while a daily dose of other one or more compositions of the kit can consist of several tablets or capsules. A kit can take the form of a dispenser designed to dispense the daily doses one at a time in the order of their intended use. The dispenser can be equipped with a memory-aid, so as to further facilitate compliance with the regimen. An example of such a memory-aid is a mechanical counter which indicates the number of daily doses that have been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

### Methods of Preparing the Compounds

The compounds disclosed herein may be prepared according the methods described in U.S. Patent Application Number 2011/0150834 and U.S. Patent Application Number 2011/0312945.

### EXAMPLE

Objective.

The objectives of the study were to assess the safety and tolerability of a range of single doses of I-32 when administered as an oral capsule to healthy subjects, and to determine the pharmacokinetic (PK) profile and pharmacodynamic (PD) effects of a range of single doses of I-32 when administered as an oral capsule to healthy subjects.

Study Design.

56 subjects were randomized 3:1 to the I-32 or placebo treatment group. Subjects received a single oral dose of I-32 or matching placebo and were followed in the Phase 1 Study Center for 48 hours following the dose. Total subject participation was between 8 and 36 days, including the Screening, Clinic, and Follow-up Periods. The following 7 dose levels of I-32 were studied: 10, 30, 100, 300, 600, 1000, and 2000 mg. Dose levels were studied sequentially beginning with the lowest dose (10 mg) and increasing stepwise until the highest dose was reached (2000 mg).

Each dose level was studied in a separate cohort of 8 subjects (6 randomized to I-32 and 2 randomized to placebo). Dosing of the 8 subjects in Cohort 1 was conducted in a staggered manner with study drug administration to 2 leading subjects (1 placebo and 1 I-32); after at least 24 hours, the remaining 6 subjects (1 placebo and 5 I-32) were dosed. If, in that 24-hour period, a dosed subject has no acute reaction, and none of the dose escalation stopping criteria were met, then the remaining 6 subjects were dosed. Subjects in Cohorts 2 and above were dosed in parallel. After each cohort has been studied and before any subsequent cohort was dosed, the safety data from the preceding cohort(s) was evaluated to ensure that the dose was safe and tolerable. Each cohort progressed through 3 distinct periods: a 1- to 27-day Screening Period, a 3-day Clinic Period, and a 5-day (±1 day) Follow-up Period.

For PD assessment of I-32, blood samples (approximately 2 mL each) were collected at Day −1 (at Check-in), 0 hours (before dose on Day 1), and at 1, 3, 8, 12, 24, and 48 hours postdose for assessment of prostaglandin D2(PGD2)-induced eosinophil responses-up regulation of cell surface CD11b.

Noncompartmental analysis was performed to determine the PD parameters of I-32.

Subject Demographics.

Subjects were planned to be male or non-pregnant female between the ages of 18 and 50 years, with body mass index (BMI) score ranged between 18.5-32.0. Subjects were to be in good health and have no clinically significant findings on a physical examination, 12-lead ECG, and clinical laboratory tests (clinical chemistry panel, complete blood count [CBC], coagulation, urine drug screen, UA).

Plasma Pharmacodynamics.

The arithmetic mean of three repeated readings for each PD sample (PGD₂-induced eosinophil response) was used for analysis. The measurement at time zero for each subject was set to baseline correspondingly. Two baseline normalized quantities were calculated: Activity/Baseline and 1-Activity/Baseline. Model 220 was called to perform the non-compartmental analysis (NCA) for the Activity/Baseline vs. Time data. Three NCA PD parameters were reported as follows:
Rₘᵢₙ Minimum observed response value (corresponding to the maximum inhibitory effect)
Tₘᵢₙ Time of Rₘᵢₙ
AUC_{Below B} Area that is below baseline and above the response curve

A new dataset was then constructed with Rₘᵢₙ as response and dose values as independent variable for each profile across the seven cohorts. An inhibitory Eₘₐₓ model (Equation 1) was fitted to the whole dataset to estimate the dose-effect relationship.

$${R}_{m i n} = {E}_{m ax} * ( 1 - \frac{Dose}{Dose + ED 50} ) \qquad Equation 1$$

### CONCLUSION

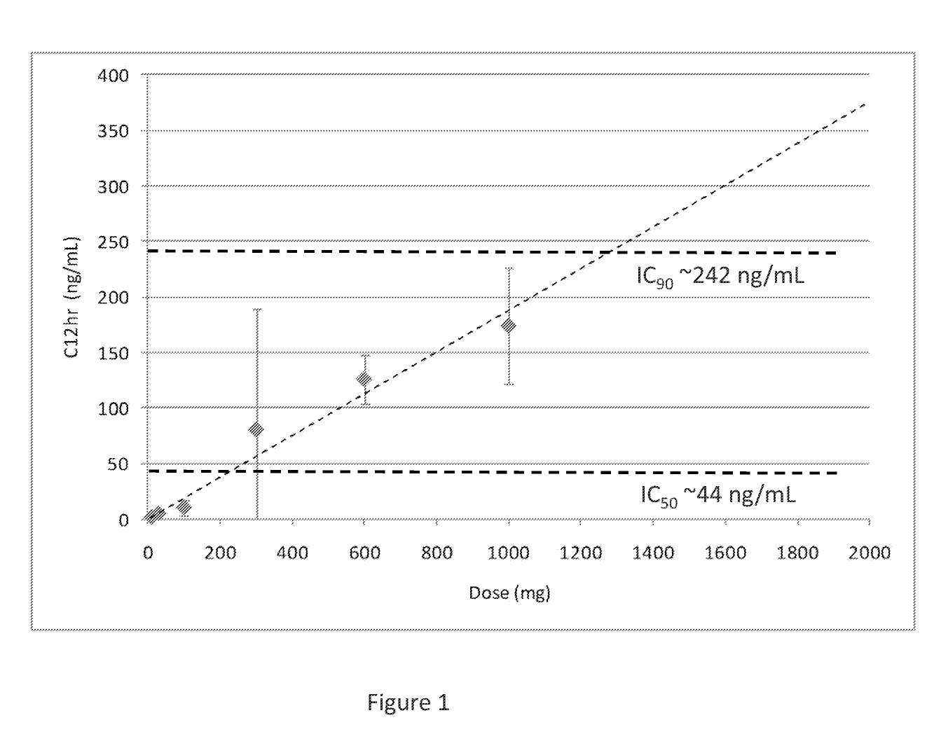

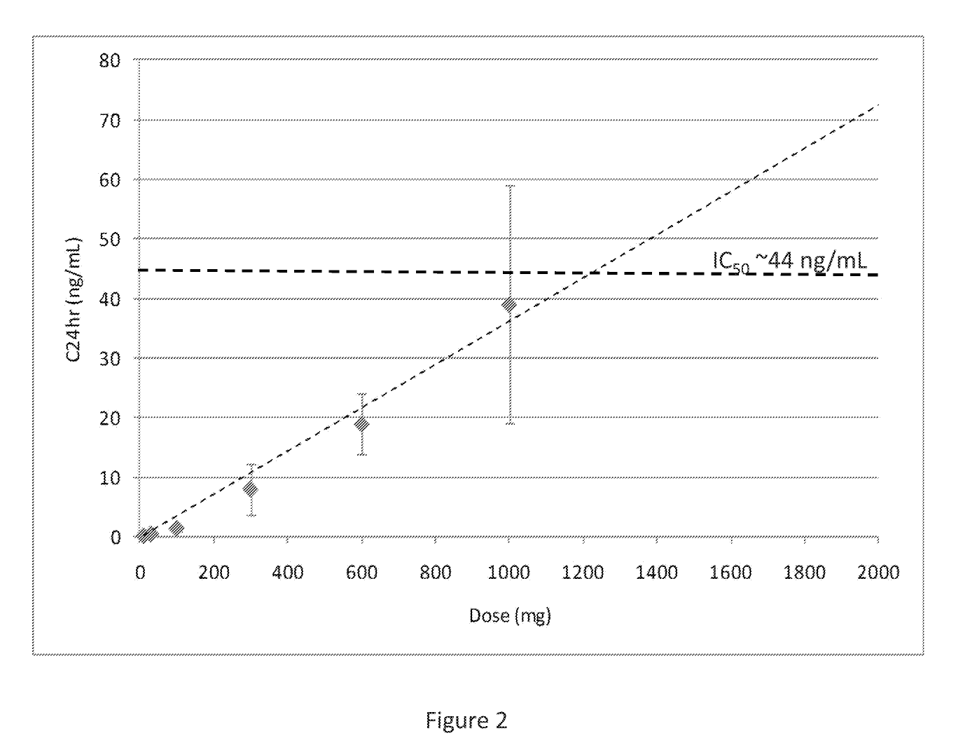

I-32 was dosed to humans in a Phase 1a, single-ascending-dose study. The Phase 1a study investigated the tolerability, safety, phamacokinetics (PK), and pharmacodynamics (PD) of single ascending doses of I-32 as oral capsules in healthy male and female volunteers. A total of 56 volunteers were randomized, with 2 receiving placebo and 6 receiving I-32 at each of the seven dose levels. The dose levels tested were 10 mg, 30 mg, 100 mg, 300 mg, 600 mg, 1000 mg, and 2000 mg. In this study, I-32 was well-tolerated at all doses. Plasma levels of I-32 were detectable in all subjects who received I-32 at any dose level. Preliminary analysis of the PD data showed dose-dependant reductions in PGD₂-mediated increase in CD11b expression on eosinophils (biomarkers), with doses of 600 mg and above showing >90% inhibition through at least 8 hours post-dose. The resulting plasma level of I-32 at the specificed dosing amounts 12 hours and 24 are shown in FIGS. 1 and 2.

A number of embodiments have been described. Nevertheless, it will be understood that various modifications may be made without departing from the spirit and scope of the invention.

We claim:

1. A method for preventing or lessening the severity of or treating a patient suffering from alopecia or acne comprising administering to said patient a therapeutically effective amount of a compound of Formula I, or a pharmaceutically acceptable salt thereof; or a pharmaceutical composition comprising the compound of Formula I, or a pharmaceutically acceptable salt thereof, and a pharmaceutically acceptable carrier; wherein the compound of Formula I is represented by the following structural formula: 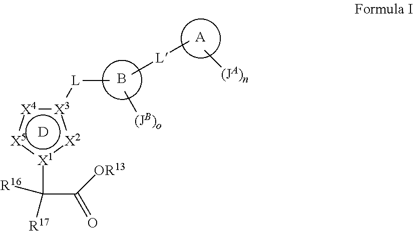
wherein:
ring A is a monocyclic or bicyclic ring selected from a 6 to 10-membered aryl, a 5 to 10-membered heteroaryl, a C₃₋₁₀ cycloaliphatic and a 4 to 10-membered heterocycle; wherein said heteroaryl or heterocycle contains from 0 to 3 ring heteroatoms independently selected from N, O and S;
ring B is a monocyclic ring selected from a phenyl and a 5 to 6-membered heteroaryl, wherein said heteroaryl contains up to three ring heteroatoms independently selected from N, O, and S;
ring D is a 5-membered heteroaryl; wherein
x¹ is selected from N and C;
x² is selected from N and C—R²;
x³ is selected from N and C;
x⁴ is selected from N and C—R⁴; and
x⁵ is selected from N and C—R⁵;
provided that at least one of x¹ or x³ is N, but both are not simultaneously N;
R² is selected from —H, a halogen, —NO₂, —CN, a C₁₋₆ aliphatic radical, a C₁₋₆ alkoxy and a cyclopropyl ring, wherein R² is independently substituted with from 0 to 3 instances of R^{A}; wherein
each R^{A} is independently selected from a halogen, —OH, a C₁₋₂ alkoxy and a C₁₋₂ haloalkoxy;
R⁴ is selected from a halogen, —NO₂, —CN, —R⁶, —OR⁶, —C(O)R⁶, —C(O)OR⁶, —N(R⁶)₂, —S(O)ₚR⁶, —S(O)₂N(R⁶)₂, —NR⁶S(O)₂R⁶, —C(O)N(R⁶)₂ and —NR⁶C(O)R⁶;
R⁵ is selected from a halogen, —NO₂, —CN, —R⁶, —OR⁶, —C(O)R⁶, —C(O)OR⁶, —N(R⁶)₂, —S(O)ₚR⁶, —S(O)₂N(R⁶)₂, —NR⁶S(O)₂R⁶, —C(O)N(R⁶)₂ and —NR⁶C(O)R⁶;
p is an integer selected from 0, 1 and 2;
each R⁶ is independently selected from —H, a C₁₋₆ aliphatic radical, and a monocyclic or bicyclic ring; wherein
the ring is selected from a 6 to 10-membered aryl, a 5 to 10-membered heteroaryl, a C₃₋₁₀ cycloaliphatic and a 4-10 membered heterocycle; wherein
when R⁶ is a C₁₋₆ aliphatic radical, it is independently substituted with from 0 to 6 instances of R⁷,
when R⁶ is a non-aromatic ring or a heteroaryl, it is independently substituted with from 0 to 6 instances of R⁸, and
when R⁶ is an aryl, it is independently substituted with from 0 to 6 instances of R⁸;
each R⁷ is independently selected from a halogen, —CN, oxo, —OR⁹, —R¹⁰, —C(O)R⁹, —C(O)OR⁹, —S(O)ₘR⁹, —N(R⁹)₂, —S(O)₂N(R⁹)₂, —NR⁹S(O)₂R⁹, —C(O)N(R⁹)₂ and —NR⁹C(O)R⁹;
each R⁸ is independently selected from a halogen, —CN, —NO₂, oxo, a C₁₋₆ aliphatic radical, —R¹⁰, —C(O)R⁹, —C(O)OR⁹, —OR⁹, —S(O)ₘR⁹, —N(R⁹)₂, —S(O)₂N(R⁹)₂, —NR⁹S(O)₂R⁹, —C(O)N(R⁹)₂ and —NR⁹C(O)R⁹;
each R^{8′} is independently selected from a halogen, —CN, —NO₂, a C₁₋₆ aliphatic radical, —R¹⁰, —C(O)R⁹, —C(O)OR⁹, —OR⁹, —S(O)ₘR⁹, —N(R⁹)₂, —S(O)₂N(R⁹)₂, —NR⁹S(O)₂R⁹, —C(O)N(R⁹)₂ and —NR⁹C(O)R⁹;
each R⁹ is independently selected from hydrogen, a C₁₋₆ aliphatic radical, and a monocyclic or bicyclic ring, wherein
the ring is selected from a 6 to 10-membered aryl, a 5 to 10-membered heteroaryl, a C₃₋₁₀ cycloaliphatic and a 4 to 10-membered heterocycle; wherein
when R⁹ is a C₁₋₆ aliphatic radical, it is independently substituted with from 0 to 6 instances of R¹¹, and
when R⁹ is a ring, it is independently substituted with from 0 to 3 instances of R¹²;
each R¹⁰ is a monocyclic or bicyclic ring independently selected from a 6 to 10-membered aryl, a 5 to 10-membered heteroaryl, a C₃₋₁₀ cycloaliphatic and a 4 to 10-membered heterocycle, and
each R¹⁰ is independently substituted with from 0 to 3 instances of R¹²;
each R¹¹ is independently selected from a halogen, —CN, —OH, a C₁₋₄ alkoxy and a C₁₋₄ haloalkoxy;
each R¹² is independently selected from a halogen, —CN, —OH, a C₁₋₄ alkyl, a C₁₋₄ haloalkyl, a C₁₋₄ alkoxy and a C₁₋₄ haloalkoxy;
R¹³ is selected from —H, a C₁₋₆ aliphatic radical, and a monocyclic or bicyclic ring, wherein the ring is selected from a 6 to 10-membered aryl, a 5 to 10-membered heteroaryl, a C₃₋₁₀ cycloaliphatic and a 4 to 10-membered heterocycle; wherein
when R¹³ is a C₁₋₆ aliphatic radical, it is independently substituted with from 0 to 6 instances of R¹⁴;
when R¹³ is a non-aromatic ring or a heteroaryl, it is independently substituted with from 0 to 6 instances of R¹⁵; and
when R¹³ is an aryl, it is independently substituted with from 0 to 6 instances of R^{15′};
each R¹⁴ is independently selected from a halogen, —CN, oxo, —OR⁹, —R¹⁰, —C(O)R⁹, —C(O)OR⁹, —S(O)ₘR⁹, —N(R⁹)₂, —S(O)₂N(R⁹)₂, —NR⁹S(O)₂R⁹, —C(O)N(R⁹)₂ and —NR⁹C(O)R⁹;
each R¹⁵ is independently selected from a halogen, —CN, —NO₂, oxo, a C₁₋₆ aliphatic radical, —R¹⁰, —C(O)R⁹, —C(O)OR⁹, —OR⁹, —S(O)ₘR⁹, —N(R⁹)₂, —S(O)₂N(R⁹)₂, —NR⁹S(O)₂R⁹, —C(O)N(R⁹)₂ and —NR⁹C(O)R⁹; and
each R^{15′} is independently selected from a halogen, —CN, —NO₂, a C₁₋₆ aliphatic radical, —R¹⁰, —C(O)R⁹, —C(O)OR⁹, —OR⁹, —S(O)ₘR⁹, —N(R⁹)₂, —S(O)₂N(R⁹)₂, —NR⁹S(O)₂R⁹, —C(O)N(R⁹)₂ and —NR⁹C(O)R⁹;
R¹⁶ and R¹⁷ are each independently selected from —H, deuterium, a C₁₋₆ alkyl, a C₁₋₆ haloalkyl and a halogen, or
alternatively, R¹⁶ and R¹⁷ are independently selected from a C₁₋₆ alkyl and a C₁₋₆ haloalkyl, and R¹⁶ and R¹⁷ taken together with the atom to which they are attached form a cyclopropyl or halocyclopropyl ring;
L is a linker selected from a methylene, —C(O)—, —O—, —S(O)ₘ— and —NR¹—; wherein
when L is a methylene, it is independently substituted with from 0 to 2 instances of R¹⁸;
m is 0, 1 or 2;
R¹ is selected from —H, a C₁₋₆ aliphatic radical, a C₃₋₆ cycloaliphatic, —CO(C₁₋₆ aliphatic), —CO(C₃₋₆ cycloaliphatic), —CO-(phenyl), a benzyl and —CO-(benzyl); wherein
when R¹ is selected from a C₁₋₆ aliphatic radical, —CO-(phenyl), a benzyl and —CO-(benzyl), it is independently substituted with from 0 to 3 instances of R^{B}; wherein
each R^{B} is independently selected from a halogen, a C₁₋₂ alkyl and a C₁₋₂ alkoxy;
each R¹⁸ is independently selected from a halogen, —CN, a C₁₋₆ aliphatic radical, a C₁₋₆ haloaliphatic radical, and a C₃₋₆ cycloaliphatic; or
alternatively, each R¹⁸ is independently selected from a C₁₋₆ aliphatic radical and a C₁₋₆ haloaliphatic radical, and two R¹⁸ groups, taken together with the atom to which they are attached form a cyclopropyl or halocyclopropyl ring;
o is an integer selected from 0, 1 and 2;
each J^{B} is independently selected from a halogen, —NO₂, —CN, —R¹⁹, —C(O)H, —C(O)OH, —C(O)NH₂, —OH, —SH, —NH₂, —C(O)R¹⁹, —C(O)OR¹⁹, —C(O)N(R²⁰)R¹⁹, —N(R²⁰)C(O)R¹⁹, —OR¹⁹, —SR¹⁹ and —NR²⁰; or
alternatively, two J^{B} groups are attached to two vicinal ring B atoms, together with said ring atoms, form a 5 to 6-membered heterocycle or a 5 to 6-membered heteroaryl, each of said rings independently substituted with from 0 to 2 instances of R^{E}, wherein each R^{E} is independently selected from a halogen, a C₁₋₂ alkyl, a C₁₋₂ alkoxy, —CN and —OH;
each R²⁰ is independently selected from a —H and a C₁₋₆ aliphatic radical;
each R¹⁹ is independently selected from a C₁₋₆ aliphatic radical, a C₃₋₆ cycloaliphatic, a phenyl, a benzyl, a 4 to 6-membered heterocycle and a 5 to 6-membered heteroaryl; wherein
when R¹⁹ is a C₁₋₆ aliphatic radical, it is independently substituted with from 0 to 3 instances of R^{C}, wherein each R^{C} is independently selected from a halogen, —CN, —OH, —NH₂, a C₃₋₄ cycloalkyl, a C₃₋₄ halocycloalkyl, a —O(C₁₋₄ alkyl), a —O(C₃₋₄ cycloalkyl), a —O(C₃₋₄ halocycloalkyl), a —O(C₁₋₄ haloalkyl), a —NH(C₁₋₄ alkyl), a —N(C₁₋₄ alkyl)₂, and —NR^{V}; wherein
—NR^{V} is a 4 to 6-membered heterocycle containing a ring N atom linked to J^{B}, and wherein said heterocycle contains from 0 to 2 additional ring heteroatoms selected from O and N;
when R¹⁹ is a heterocycle or a heteroaryl it contains from 1 to 3 ring heteroatoms independently selected from N, O and S;
when R¹⁹ is a phenyl, it is independently substituted with from 0 to 3 instances of R^{D}, wherein each R^{D} is independently selected from a halogen, a C₁₋₄ aliphatic radical, —CN, —OH, —NH₂, a —O(C₁₋₄ alkyl), a —NH(C₁₋₄ alkyl) and a —N(C₁₋₄ alkyl)₂; and
when R¹⁹ is a non-aromatic ring or a heteroaryl, it is independently substituted with from 0 to 3 instances of R^{D′}, wherein each R^{D}, is independently selected from a halogen, oxo, a C₁₋₄ aliphatic radical, —CN, —OH, —NH₂, a —O(C₁₋₄ alkyl), a —NH(C₁₋₄ alkyl) and a —N(C₁₋₄ alkyl)₂;
L′ is a linker selected from —Y—SO₂—, —NR²¹SO₂—, —SO₂NR²¹—, —Y—C(O)—, —NR²¹C(O)— and —C(O)NR²¹—; wherein
Y is selected from a single bond, a straight C₁₋₂ alkylene linker, and a branched C₂ alkylene linker, wherein the C₁₋₂ alkylene linker is independently substituted with from 0 to 3 halogen atoms;
R²¹ is selected from hydrogen, a C₁₋₆ alkyl, a C₁₋₆ haloalkyl and a C₃₋₆ cycloalkyl ring;
n is an integer selected from 0, 1, 2 and 3;
each J^{A} is independently selected from a halogen, —NO₂, —CN, —R²², —C(O)H, —C(O)OH, —C(O)NH₂, —OH, —SH and —NH₂, —C(O)R²², —C(O)OR²², —C(O)N(R²³)R²², —N(R²³)C(O)R²², —OR²², —SR²² and —NR²²R²³;
each R²³ is independently selected from a —H and a C₁₋₆ aliphatic radical;
each R²² is independently selected from a C₁₋₆ aliphatic radical, a C₃₋₆ cycloaliphatic ring, a phenyl, a benzyl, a 4 to 6-membered heterocycle and a 5 to 6-membered heteroaryl; wherein
when R²² is a C₁₋₆ aliphatic radical, it is independently substituted with from 0 to 3 instances of R^{F}, wherein each R^{F} is independently selected from a halogen, —CN, —OH, —NH₂, a C₃₋₄ cycloalkyl, a C₃₋₄ halocycloalkyl, a —O(C₁₋₄ alkyl), a —O(C₃₋₄ cycloalkyl), a —O(C₃₋₄ halocycloalkyl), a —O(C₁₋₄ haloalkyl), a —NH(C₁₋₄ alkyl), a —N(C₁₋₄ alkyl)₂ and —NR^{V}; wherein
—NR^{V} is a 4 to 6-membered heterocycle containing a ring N atom linked to J^{B}, and wherein the heterocycle contains from 0 to 2 additional ring heteroatoms selected from O and N;
when R²² is a heterocycle or a heteroaryl, the ring contains from 1 to 3 ring heteroatoms independently selected from N, O and S;
when R²² is a non-aromatic ring or a 5 to 6-membered heteroary, it is independently substituted with from 0 to 3 instances of R^{G}, wherein
each R^{G} is independently selected from a halogen, oxo, a C₁₋₄ aliphatic radical, —CN, —OH, —NH₂, a —O(C₁₋₄ alkyl), a —NH(C₁₋₄ alkyl) and a —N(C₁₋₄ alkyl)₂; and
when R²² is a phenyl, it is independently substituted with from 0 to 3 instances of R^{G′}, wherein
each R^{G′} is independently selected from a halogen, a C₁₋₄ aliphatic radical, —CN, —OH, —NH₂, —O(C₁₋₄ alkyl), —NH(C₁₋₄ alkyl) and —N(C₁₋₄ alkyl)₂.

2. The method of claim 1, wherein R¹⁶ and R¹⁷ are each independently selected from —H and a methyl or, alternatively, R¹⁶ and R¹⁷ taken together with the carbon to which they are attached, form a cyclopropyl ring.

3. The method according to claim 2, wherein R¹⁶ and R¹⁷ are both —H.

4. The method according to any one of claims 1-3, wherein ring D is selected from a pyrrole, a pyrazole and an imidazole.

5. The method according to claim 4, wherein ring D is a pyrrole, and x¹ or x³ is N.

6. The method according to claim 5, wherein ring D is a pyrrole, and x¹ is N.

7. The method according to claim 6, wherein the compound is selected from compounds having formula IA or formula IB:

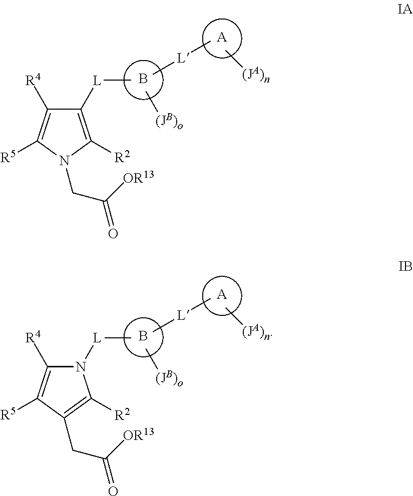

8. The method according to any one of claims 1-7, wherein ring B is selected from a phenyl, a thiophene and a 6-membered heteroaryl.

9. The method according to claim 8, wherein ring B is selected from a phenyl, a thiophene and a pyridine.

10. The method according to claim 9, wherein ring B is a phenyl.

11. The method according to any one of claims 1-10, wherein L is selected from a methylene, —C(O)— and —S—.

12. The method according to claim 11, wherein L is selected from a methylene and —S—.

13. The method according to claim 12, wherein the compound is selected from compounds having formula IIA or formula IIB:

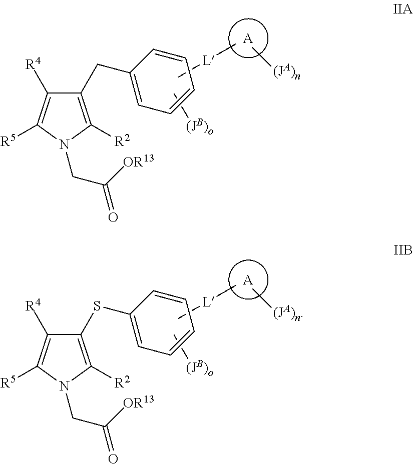

14. The method of any one of claims 1-13, wherein R² is selected from a halogen, —H, a cyclopropyl ring, a C₁₋₄ alkyl and a C₁₋₄ haloalkyl.

15. The method according to claim 14, wherein R² is selected from a C₁₋₄ alkyl and —H.

16. The method according to claim 15, wherein R² is a methyl.

17. The method according to claim 16, wherein the compound is selected from compounds having structural formula IIIA or structural formula IIIB:

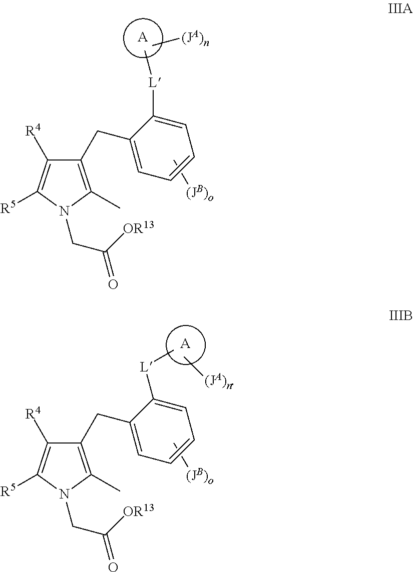

18. The method according to any one of claims 1-17, wherein L′ is selected from —SO₂— and —CH₂SO₂—.

19. The method according to claim 18, wherein L′ is —SO₂—.

20. The method of any one of claims 1-19, wherein o is 0.

21. The method according to claim 20, wherein the compound is selected from compounds having structural formula IVA or structural formula IVB:

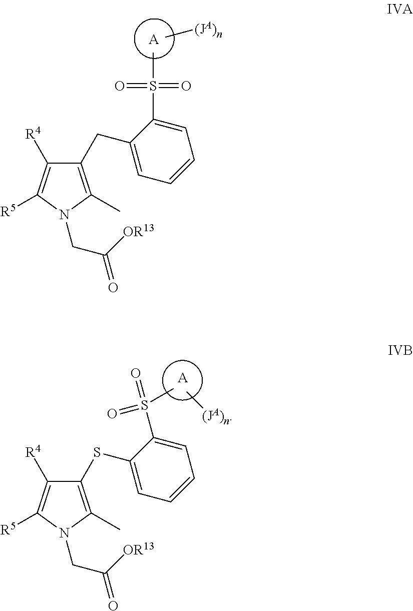

22. The method of any one of claims 1-21, wherein ring A is selected from a phenyl, a 5 to 6-membered heteroaryl, a C₃₋₆ cycloaliphatic and a 5 to -membered heterocycle, wherein said heteroaryl or heterocycle contains from 1 to 2 ring heteroatoms selected from N and O.

23. The method of claim 22, wherein ring A is selected from a phenyl and a 5 to 6-membered heterocyclic ring, wherein said heterocycle contains from 1 to 2 ring heteroatoms selected from O and N.

24. The method of claim 23, wherein ring A is selected from a phenyl, a pyridine, a thiophene, a furan, a pyrimidine, a pyrazine, a piridazine, a piperidine, a piperazine, a morpholine and a pyrrolidine.

25. The method of claim 24, wherein ring A is selected from a phenyl, a morpholine and a pyrrolidine.

26. The method of claim 25, wherein ring A is selected from a phenyl, an N-linked morpholine and an N-linked pyrrolidine.

27. The method of claim 26, wherein the compound is selected from compounds having structural formula VA, VB, VC, VD, VE or VF:

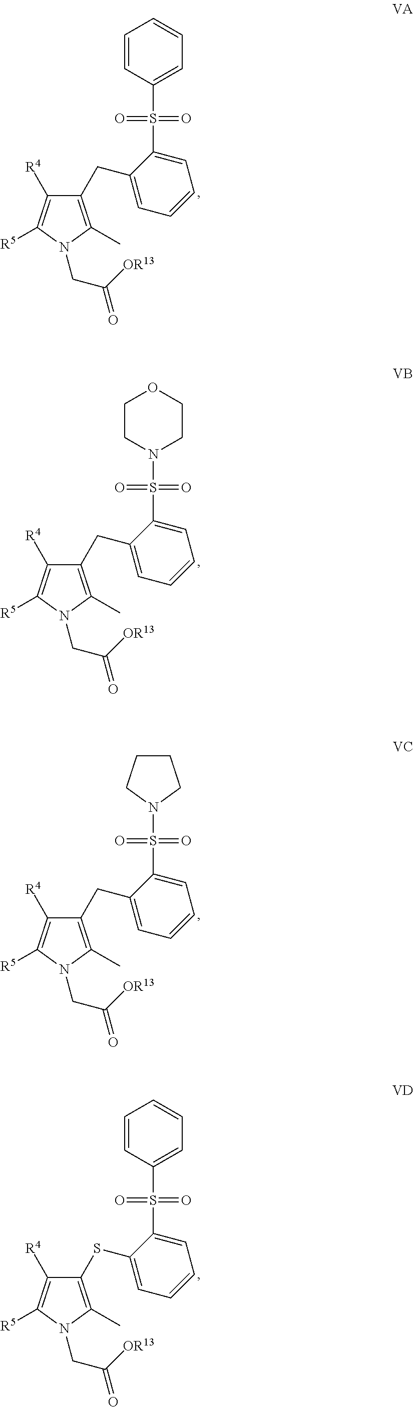

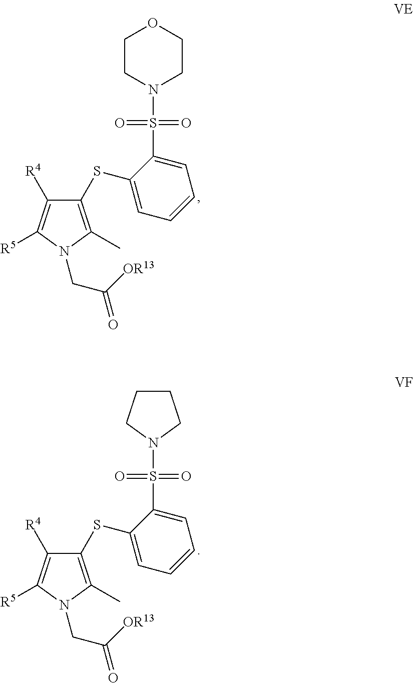

28. The method of any one of claims 1-27, wherein R⁴ is selected from a halogen, —NO₂, —R⁶, —OR⁶, —C(O)R⁶, —C(O)OR⁶, —N(R⁶)₂, —S(O)ₚR⁶, —S(O)₂N(R⁶)₂, —NR⁶S(O)₂R⁶, —C(O)N(R⁶)₂ and —NR⁶C(O)R⁶.

29. The method of claim 28, wherein R⁴ is selected from a —H, a halogen, —CN, a C₁₋₆ aliphatic radical, a C₃₋₆ cycloaliphatic ring, a C₁₋₆ haloaliphatic radical, a phenyl which is optionally substituted by R^{8′}, a benzyl which is optionally substituted by R^{8′}, —OR⁶ and —C(O)R⁶.

30. The method of claim 29, wherein R⁴ is selected from —H, a halogen, —CN, a C₁₋₄ alkyl, a C₁₋₄ haloalkyl, a C₃₋₆ cycloalkyl, a —O(C₁₋₄ alkyl), a —O(C₁₋₄ haloalkyl), a —O(C₃₋₆ cycloalkyl), a —O(phenyl), a —O(substituted phenyl), a —O(benzyl), a —O(substituted benzyl), a —C(O)(C₁₋₄ alkyl), a —C(O)(C₁₋₄ haloalkyl), a —C(O)(C₃₋₆ cycloalkyl), a —C(O)(phenyl), a —C(O)(substituted phenyl), a —C(O)(benzyl), —C(O)(substituted benzyl) and —C(O)H; wherein each of said substituted phenyl or benzyl rings, is substituted by from 0 to 4 instances of R^{8′};

31. The method of claim 30, wherein R⁴ is selected from —H, a halogen, —CN, an ethyl, a methyl, a propyl, a trifluoroethyl, a trifluoromethyl, a cyclopropyl, a cyclopentyl, a cyclohexyl, a cyclopropyloxy, a cyclopentyloxy, a cyclohexyloxy, an ethoxy, a methoxy, a propyloxy, a trifluoromethoxy, a trifluoroethoxy, a benzoyl, a phenyl, a phenyloxy, a methylcarbonyl, an ethylcarbonyl, a trifluoromethylcarbonyl, a trifluoroethylcarbonyl, and —C(O)H; wherein each of said benzoyl, phenyl or phenyloxy is independently substituted by from 0 to 4 instances of R^{8′}.

32. The method of claim 31, wherein R⁴ is selected from a —H, a halogen, —CN, an ethyl, a methyl, a propyl, a trifluoroethyl, a trifluoromethyl, a cyclopropyl, a cyclopentyl, a cyclohexyl, phenyl, a benzoyl, a methylcarbonyl, an ethylcarbonyl, a trifluoromethylcarbonyl, a trifluoroethylcarbonyl and —C(O)H; wherein each of said phenyl and benzoyl groups is independently substituted by from 0 to 4 instances of R^{8′}.

33. The method of claim 32, wherein R⁴ is selected from a —H, iodo, —CN, methyl, 2,2,2-trifluoroethyl, benzoyl, methylcarbonyl, trifluoromethylcarbonyl, —C(O)H and phenyl; wherein said phenyl is independently substituted with from 0 to 2 instances of halogen.

34. The method of claim 33, wherein R⁴ is a phenyl substituted with from 0 to 2 instances of halogen.

35. The method of claim 34, wherein R⁴ is a phenyl substituted with from 0 to 2 instances of fluoro.

36. The method of claim 33, wherein R⁴ is selected from a —H, —CN, a methyl, 2,2,2-trifluoroethyl, a benzoyl, a methylcarbonyl, a trifluoromethylcarbonyl, —C(O)H, a phenyl and a fluorophenyl; wherein said fluorophenyl is substituted with from 0 to 2 additional instances of fluoro.

37. The method of any one of claims 1-36, wherein R⁵ is selected from a halogen, —CN, a C₁₋₆ aliphatic radical independently substituted with from 0 to 4 instances of R⁷, a C₃₋₆ cycloaliphatic, a phenyl independently substituted with from 0 to 4 instances of R^{8′}, and a 6-membered heteroaryl independently substituted with from 0 to 4 instances of R^{8′}.

38. The method of claim 37, wherein R⁵ is selected from a halogen, —CN, a C₁₋₆ alkyl independently substituted with from 0 to 4 instances of R⁷, a C₃₋₆ cycloaliphatic, a phenyl independently substituted by from 0 to 4 instances of R^{8′}, and a 6-membered heteroaryl independently substituted by from 0 to 4 instances of R^{8′}.

39. The method of claim 38, wherein R⁵ is selected from the group consisting of: a halogen, —CN; a C₁₋₆ alkyl substituted with from 0 to 2 instances of a substituent independently selected from halogen and —OH; a 3-6 membered cycloalkyl, a phenyl and a 6-membered heteroaryl; wherein each of said phenyl and 6-membered heteroaryl rings is substituted by from 0 to 3 instances of a substituent independently selected from a halogen, a C₁₋₄ alkyl, a C₁₋₄ haloalkyl, a C₁₋₄ alkoxy, a C₁₋₄ haloalkoxy and —CN.

40. The method of claim 39, wherein R⁵ is selected from a halogen, —CN, an ethyl, a methyl, a propyl, a 3-6 membered cycloalkyl, a phenyl, a pyridine and a pyrimidine; wherein each said methyl, ethyl and propyl is independently substituted with from 0 to 4 instances of a halogen or —OH; and wherein each said phenyl, pyridine and pyrimidine is substituted with from 0 to 4 instances of a substituent independently selected from a halogen, a C₁₋₂ alkyl, a C₁₋₂ haloalkyl, an C₁₋₂ alkoxy and a C₁₋₂haloalkoxy.

41. The method of claim 40, wherein R⁵ is selected from —CN, an ethyl, a methyl, a propyl, a cyclopropyl, a cyclopentyl, a cyclohexyl, a phenyl and a pyridine; wherein each said methyl, propyl and ethyl is independently substituted with from 0 to 2 instances of a halogen or —OH; wherein said phenyl is independently substituted by from 0 to 2 instances of halogen or —CF₃; and wherein said pyridine is substituted by from 0 to instances of a substituent independently selected from a halogen, a C₁₋₂ alkoxy, a C₁₋₂ haloalkoxy and CF₃.

42. The method of claim 41, wherein R⁵ is selected from a —CN, a 2-hydroxyethyl, a methyl, a cyclopropyl, a cyclopentyl, a cyclohexyl, a phenyl and a pyridine; wherein said phenyl is independently substituted by from 0 to 2 instances of fluorine or —CF₃; and wherein said pyridine is independently substituted by from 0 to 1 instances of fluoro or chloro.

43. The method of claim 42, wherein R⁵ is selected from —CN, a methyl, a cyclopropyl, a cyclopentyl, a cyclohexyl, a phenyl, pyridine, a 3-chloro-4-pyridinyl and a 3-chloro-2-pyridinyl; wherein said phenyl is independently substituted by from 0 to 2 instances of fluorine or from 0 to 1 instances of —CF₃.

44. The method according to any one of claims 1-4, wherein ring D is a pyrazole, and x¹ and x² are both N.

45. The method of any one of claims 1-44, wherein R¹³ is selected from a —H and a C₁₋₆ alkyl.

46. The method of claim 45, wherein R¹³ is a —H.

47. The method of any one of claims 1-19, wherein o is 1 or 2 and J^{B} is a halogen.

48. A method selected from those depicted in Table 1.

49. The method of claim 1, wherein the compound having Formula I is not a compound selected from 5-[[6-methoxy-3-(4-methoxybenzoyl)-2-methyl-1H-pyrrolo[2,3-b]pyridin-1-yl]methyl]-α,α-dimethyl-2H-Tetrazole-2-acetic acid [CAS Registry No. 1097838-63-5], 5-[[5-(benzoylamino)-2-thiazolyl]thio]-2H-tetrazole-2-acetic acid [CAS Registry No. 1099441-56-1], 2-butyl-1-[[4-[(2-carboxybenzoyl)amino]phenyl]methyl]-5-chloro-1H-imidazole-4-acetic acid [CAS Registry No. 114798-40-2], and 2-butyl-1-[[4-[(2-carboxybenzoyl)amino]phenyl]methyl]-5-chloro-1H-imidazole-4-acetic acid [CAS Registry No. 114773-45-4], or a pharmaceutically acceptable salt thereof.

50. A method for preventing or lessening the severity of or treating a patient suffering from alopecia or acne comprising administering to said patient a therapeutically effective amount of a compound of Formula II, or a pharmaceutically acceptable salt thereof; or a pharmaceutical composition comprising the compound of Formula II, or a pharmaceutically acceptable salt thereof, and a pharmaceutically acceptable carrier; wherein the compound of Formula II is represented by the following structural formula: 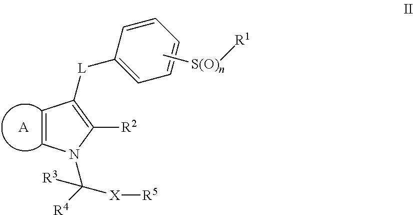
wherein
A is a 5, 6, 7, 8, 9 or 10-membered non-aromatic carbocycle; wherein A is optionally substituted with up to eight instances of R⁸;
L is chosen from
—O—,
—S(O)ₘ—,
—NR¹⁴— and
(C₁-C₃)alkylene, wherein, when said (C₁-C₃)alkylene is a C₂- or C₃-alkylene, one CH₂ is optionally replaced by —O—, —S(O)ₘ— or —NR¹⁴—, and wherein one or more substitutable carbon atoms of said (C₁-C₃)alkylene is optionally substituted with up to three instances of R¹¹;
X is chosen from a direct bond and (C₁-C₂)alkylene, wherein said (C₁-C₂)alkylene is optionally substituted with up to two instances of R¹²;
R¹ is chosen from (3-8-membered)carbocyclyl, (3-8-membered) heterocyclyl, —NR⁶(C₁-C₆)alkyl, —NR⁶(3-8-membered)carbocyclyl and —NR⁶(3-8-membered)heterocyclyl;
wherein R¹ is optionally substituted with up to four instances of R⁹;
R² is chosen from hydrogen, halogen, (C₁-C₄)alkyl, (C₁-C₄)haloalkyl, and OH;
R³ is chosen from hydrogen, halogen, (C₁-C₄)alkyl and (C₁-C₄)haloalkyl;
R⁴ is chosen from hydrogen, halogen, (C₁-C₄)alkyl and (C₁-C₄)haloalkyl; or,
R³ and R⁴, taken together, form a (C₃-C₇)cycloalkyl ring;
R⁵ is chosen from C(O)OR⁷, C(O)N(R⁷)₂, C(O)NOR⁷ and C(O)NSR⁷;
R⁶ is chosen from hydrogen and (C₁-C₆)alkyl;
R⁷ is selected from hydrogen and (C₁-C₄)alkyl, wherein said (C₁-C₄)alkyl is optionally substituted with up to four instances of R¹⁶;
R⁸ in each occurrence is independently selected from halogen, (C₁-C₄)alkyl, (C₁-C₄)haloalkyl, (C₁-C₄)alkoxy, (C₁-C₄)haloalkoxy, CN, OH, oxo and N(R¹⁴)₂;
R⁹ in each occurrence is independently selected from halogen, (C₁-C₄)alkyl, (C₁-C₄)haloalkyl, (C₁-C₄)alkylcarbonyl, (C₁-C₄)alkoxycarbonyl, CN, OH and N(R¹⁴)₂;
R¹⁰ in each occurrence is independently selected from halogen, (C₁-C₄)alkyl, (C₁-C₄)haloalkyl, (C₁-C₄)alkylcarbonyl, (C₁-C₄)alkoxycarbonyl, CN, OH and N(R¹⁵)₂;
R¹¹ in each occurrence is independently selected from halogen, (C₁-C₄)alkyl, (C₁-C₄)haloalkyl, (C₃-C₆)cycloalkyl, CN, OH, (C₁-C₄)alkoxy and (C₁-C₄)haloalkoxy;
R¹² in each occurrence is independently selected from halogen, (C₁-C₄)alkyl and (C₁-C₄)haloalkyl;
R¹⁴ is selected from hydrogen and (C₁-C₄)alkyl, wherein said (C₁-C₄)alkyl is optionally substituted with up to four instances of R¹⁰;
R¹⁵ is selected from hydrogen and (C₁-C₄)alkyl;
R¹⁶ in each occurrence is independently selected from halogen, (C₁-C₄)alkyl, (C₁-C₄)haloalkyl, (C₁-C₄)alkylcarbonyl, (C₁-C₄)alkoxycarbonyl, (3-8-membered)carbocyclyl, (3-8-membered) heterocyclyl, CN, OH and N(R¹⁵)₂;
m is zero, one or two; and
n is zero, one or two.

51. The method of claim 50, wherein R¹⁶ in each occurrence is independently selected from halogen, (C₁-C₄)alkyl, (C₁-C₄)haloalkyl, (C₁-C₄)alkylcarbonyl, (C₁-C₄)alkoxycarbonyl, CN, OH and N(R¹⁵)₂.

52. The method according to any one of claim 50 or 51, wherein A is a fused cycloheptyl ring optionally substituted with up to eight instances of R⁸, independently selected, wherein said compound has the formula: 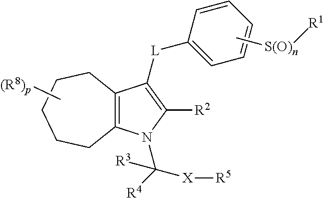
and p is zero or an integer from 1 to 8.

53. The method according to any one of claim 50 or 52, wherein A is a fused cyclohexyl ring optionally substituted with up to eight instances of R⁸, independently selected, wherein said compound has the formula: 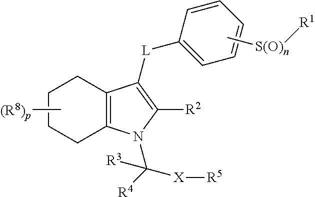
and p is zero or an integer from 1 to 8.

54. The method of claim 53 wherein two R⁸ are each a methyl residue attached to the same ring carbon.

55. The method of claim 54 of formula 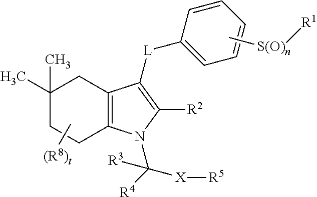
wherein t is zero or an integer from 1 to 4.

56. The method of claim 55, wherein t is zero.

57. The method of claim 53, wherein the fused cyclohexyl ring is substituted with up to eight instances of R⁸ independently selected from the group consisting of halogen and (C1-C4)alkyl.

58. The method of claim 57 having one of the following two formulae: 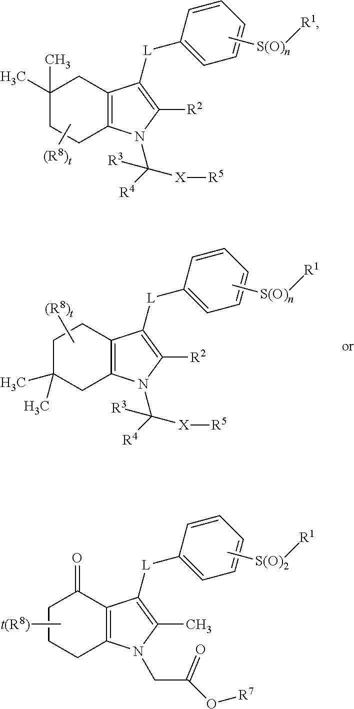
wherein t is 0 or an integer of from 1 to 6.

59. The method of claim 58, wherein the up to six instances of R⁸ are independently selected from the group consisting of fluoro and methyl.

60. The method of claim 59, wherein t is 2 or 4.

61. The compound of claim 53 having the formula: 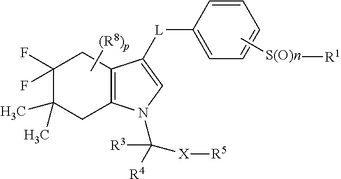
and p is zero or an integer from 1 to 4.

62. The method of claim 61 wherein p is zero or R⁸ is an oxo.

63. The method of claim 50 or 51 wherein A is a fused cyclopentyl ring optionally substituted with up to six instances of R⁸, independently selected, wherein said compound has the formula: 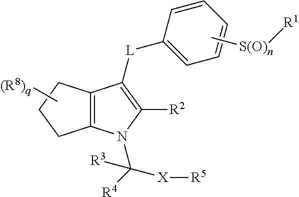
and q is zero or an integer from 1 to 6.

64. The method of any one of claims 50-63, wherein X is chosen from a direct bond, —CH₂— and —CH₂CH₂—.

65. The method of claim 64, wherein X is a direct bond.

66. The method of any one of claims 50-63 wherein R² is chosen from hydrogen, fluoro, methyl, ethyl and trifluoromethyl.

67. The method of claim 66 wherein R² is methyl.

68. The method of any one of claims 50-63 wherein R³ and R⁴ are taken together to form a cyclopropyl ring.

69. The method of any one of claims 50-63, wherein R³ and R⁴ are each independently selected from hydrogen and methyl, wherein said methyl is optionally substituted with 1-3 instances of halogen.

70. The method of claim 69, wherein said halogen is fluoro.

71. The method of claim 70, wherein R³ and R⁴ are each hydrogen.

72. The method of any one of claims 50-63, wherein L is —CH₂—, —O— or —S(O)ₘ—.

73. The method of claim 72 wherein L is —CH₂—.

74. The method of any of claims 50-63 wherein L is —S(O)ₘ— and m is 2.

75. The method according to any one of claims 50-63, wherein X is a direct bond and R⁵ is C(O)OR⁷, C(O)N(R⁷)₂, C(O)NHOR⁷ or C(O)NHSR⁷, wherein R⁷ is H or (C₁₋₄)alkyl.

76. The method of claim 71, wherein R⁵ is C(O)OH or C(O)O(C₁₋₄)alkyl.

77. The method of claim 76, wherein R⁵ is C(O)OH.

78. The method of any one of claims 50-63, wherein R¹ is a non-aromatic 3-8 membered heterocycle, phenyl, or a non-aromatic 3-8 membered carbocycle, wherein R¹ is optionally substituted with up to four instances of R⁹.

79. The method of claim 78, wherein R¹ is phenyl or a non-aromatic 3-8 membered carbocycle, wherein R¹ is optionally substituted with up to four instances of R⁹.

80. The method of claim 79, wherein R¹ is a non-aromatic 3-8 membered heterocycle, wherein R¹ is optionally substituted with up to four instances of R⁹.

81. The compound of claim 80, wherein R¹ is a non-aromatic 5-7 membered heterocycle, wherein R¹ is optionally substituted with one to three instances of R⁹.

82. The compound of claim 81 wherein R¹ is an N-attached pyrrolidine, piperidine, piperazine, azepine or morpholine, optionally substituted with one to three instances of R⁹, wherein each R⁹ is independently selected from (C₁-C₄)alkyl and (C₁-C₄)haloalkyl.

83. The method of claim 82, wherein R¹ is an N-attached piperazine of formula 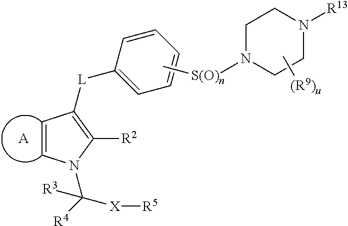
wherein R¹³ is chosen from hydrogen, (C₁-C₄)alkyl, (C₁-C₄)alkylcarbonyl and (C₁-C₄)alkoxycarbonyl and u is zero, one or two.

84. The method of claim 82 wherein R¹ is an N-attached morpholine of formula 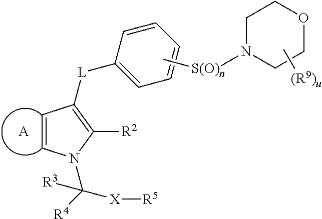
wherein u is zero, one or two.

85. The method of any one of claims 50-63 wherein —S(O)ₙR¹ is attached para or ortho to L on the phenyl ring.

86. The method of claim 85, wherein said compound has one of the following formulae: 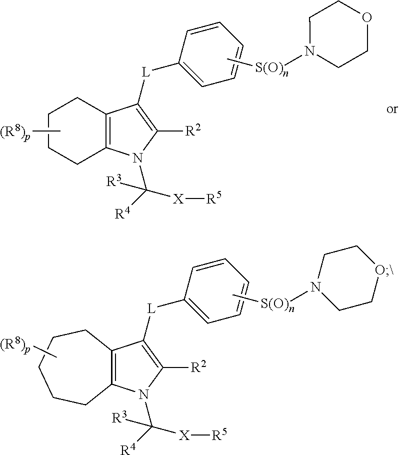
wherein p is zero or an integer from 1 to 3.

87. The method of claim 86, wherein said compound has the following formulae: 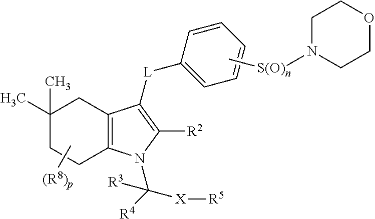
wherein p is zero or one.

88. The method of claim 87 wherein —S(O)ₙR¹ is attached para or ortho to L on the phenyl ring.

89. The method of claim 88, wherein X is a direct bond and R⁵ is C(O)OH or C(O)O(C₁₋₄)alkyl.

90. The method of any one of claims 50-63 wherein R¹ is —NR⁶(C₁-C₆)alkyl and R⁶ is hydrogen or methyl.

91. The method of claim 50 or 51 wherein
A is chosen from a cyclopentyl ring, a cycloheptyl ring and a dimethylcyclohexyl ring;
R² is methyl;
R³ and R⁴ are hydrogen;
L is —CH₂—;
n is 2; and
R¹ is chosen from
(a) pyrrolidinyl, piperidinyl, azepinyl or morpholinyl;
(b) piperazine-1-yl substituted at 4 with (C₁-C₄)alkylcarbonyl or (C₁-C₄)alkoxycarbonyl;
(c) —NR⁶(C₁-C₆)alkyl wherein R⁶ is hydrogen or methyl; and
(d) phenyl and substituted phenyl.

92. The method of claim 50 which has the formula of III 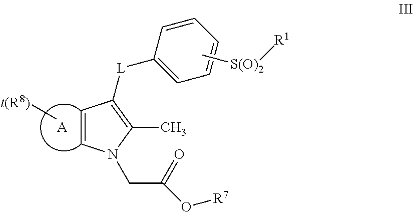
in which
A is chosen from a cyclopentyl ring, a cycloheptyl ring, a cyclohexyl ring, and a dimethylcyclohexyl ring;
R⁷ is hydrogen or (C₁₋₄) alkyl;
L is —CH₂— or —S(O)ₘ—;
m is 0 or 2;
R⁸ is oxo;
t is 0, 1 or 2; and
R¹ is chosen from
(a) pyrrolidinyl, piperidinyl, azepinyl or morpholinyl;
(b) piperazine-1-yl substituted at the 4 position with (C₁-C₄)alkylcarbonyl or (C₁-C₄)alkoxycarbonyl;
(c) —NR⁶(C₁-C₆)alkyl wherein R⁶ is hydrogen or methyl; and
(d) phenyl and substituted phenyl.

93. The method of claim 92, wherein A is a cyclohexyl ring or a dimethylcyclohexyl ring.

94. The method of claim 93, wherein the compound has the formula of III′ or III″:

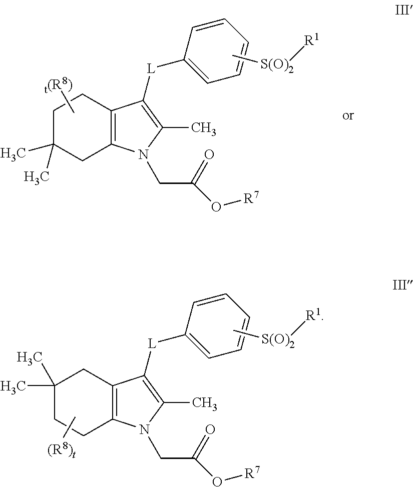

95. The method of any one of claims 92-94, wherein L is —CH₂—.

96. The method of any one of claims 92-95, wherein t is one and R⁸ is oxo.

97. The method of any one of claims 93-96, wherein the compound has the formula of IV:

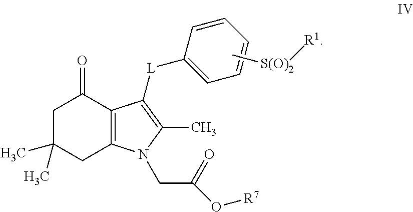

98. The method of any one of claims 93-97, wherein R¹ is chosen from pyrrolidine, piperidinyl, azepinyl, morpholinyl or phenyl.

99. The method according to claim 98, wherein R¹ is chosen from pyrrolidine or morpholine.

100. The method according to any one of claims 93-98, wherein —SO)₂R¹ is attached para or ortho to L on the phenyl ring.

101. The method according to any one of claims 1-100, for treating alopecia.

102. The method according to claim 101 wherein the alopecia is androgenetic alopecia.

103. The method according to claim 101 wherein the alopecia is alopecia greata.

104. The method according to any one of claims 1-100, for treating acne.

105. The method according to claim 104, wherein the acne is acne vulgaris.

106. The method according to claim 104, for treating cystic acne.

107. The method according to any one of claims 1-106, wherein the compound is administered once a day.

108. The method according to any one of claims 1-106, wherein the compound is administered twice a day.

109. The method according to any one of claims 1-108, wherein the compound is administered orally.

110. The method according to any one of claims 1-108, wherein the compound is administered parenterally.

111. The method according to any one of claims 1-108, wherein the compound is administered topically.

112. The method according to any one of claims 1-111, wherein the compound is administered at a dose of about 200 to about 1400.

113. The method according to any one of claims 1-111, wherein the compound is administered at a dose of about 200 to 500 mg.

114. The method according to any one of claims 1-111, wherein the compound is administered at a dose of about 400 to 700 mg.

115. The method according to any one of claims 1-111, wherein the compound is administered at a dose of about 600 to 900 mg.

116. The method according to any one of claims 1-111, wherein the compound is administered at a dose of about 800 to 1100 mg.

117. The method according to any one of claims 1-111, wherein the compound is administered at a dose of about 1000 to 1300 mg.
